(19) United States
(12) Patent Application Publication
(10) Pub. No.: US 2018/0311358 A1
(43) Pub. Date: Nov. 1, 2018

(54) THERMOFORMABLE DUAL NETWORK HYDROGEL COMPOSITIONS

(71) Applicant: Lubrizol Advanced Materials, Inc., Cleveland, OH (US)

(72) Inventors: Nancy S. Marchant, Medina, OH (US); James R. Baxter, Macedonia, OH (US); William A. Stansbrey, Hudson, OH (US)

(21) Appl. No.: 15/773,643

(22) PCT Filed: Nov. 2, 2016
(86) PCT No.: PCT/US16/60054

§ 371 Date: May 4, 2018

Related U.S. Application Data
(60) Provisional application No. 62/251,345, filed on Nov. 5, 2015.

(51) Int. Cl. A61K 47/10; A61K 47/32; A61K 47/34; A61K 47/14; A61K 9/70; A61K 9/00

(52) U.S. Cl. CPC A61K 47/10; A61K 47/32; A61K 47/34; A61L 15/225; A61K 9/7023; A61K 9/006; A61K 47/14

(57) ABSTRACT

A thermoformable dual network hydrogel composition formed from a cross-linked polymer derived from one or more olefinically unsaturated polymerizable carboxylic monomers and one or more thermoplastic polyurethane compositions is disclosed. The hydrogel exhibits high elastic modulus and Yield Stress at low shear, and a moderate but significant adhesion force. The hydrogel composition provides useful materials for personal care, health care, medical and pharmaceutical applications, among others.

### FIELD OF THE INVENTION

The invention relates to a hydrogel composition formed from a pH sensitive microgel cross-linked poly(acrylic) acid and a water soluble thermoplastic polyurethane. The hydrogels of the invention resist deformation at room temperature yet can be thermoformed and are water and electrolyte resistant at room and body temperature. The hydrogel can be employed in applications where gentle adhesion, high conformability, and a high water environment are beneficial such as wound dressings controlled drug delivery devices, micro fluidic devices, biosensors and for dermal, mucosal and transdermal delivery of chemically and physiologically active ingredients in personal care, health care, and pharmaceutical applications.

### BACKGROUND OF THE INVENTION

Hydrogels are soft polymers generally composed of about 75% to 99% water having a wide range of potential applications, including, among others, advanced membranes, regenerative medicine and wound care. Most conventional hydrogels are chemically crosslinked and commonly referred to as thermosets. The network of polymer in the water once formed cannot un-form and the hydrogel is confined to one shape i.e. the shape formed when the gel was chemically formed. These hydrogels suffer from a number of limitations. High water content hydrogels without sufficient network structure are often soft and lack compression resistance. High polymer content hydrogels become brittle and fragment easily.

Various solutions have been proposed to overcome these drawbacks in materials. For instance, it is well known that rubber may be reinforced to increase its toughness, where brittle materials may be blended with soft materials to increase the strength of the soft material. Similar concepts may be applied to hydrogels. It has been known in the art to blend rigid brittle network hydrogels with soft ductile network hydrogels by chemically crosslinking to provide improved properties. Such compositions, however, are not able to be molded with heat, compression molded, or thermoformed and thus not able be molded to a very different shape once the dual network is chemically formed.

There is currently a need for polymeric compositions having controlled nanometer-scale structural and mechanical integrity as well as a need for improved methods of making these polymer compositions at moderate temperature and high water environments. There is also a need for improved polymeric compositions for use as wound covering and/or skin soothing, and for delivery of chemically and physiologically active ingredients in personal potential biomedical applications in controlled drug delivery devices, micro fluidic devices, biosensors and advanced membranes care, health care, and pharmaceutical applications and other.

### SUMMARY OF THE INVENTION

The disclosed technology provides a dual network semi-solid hydrogel composition that is capable of being formed with heat, compression molded, or thermoformed and exhibiting increased durability and robustness. The gel as disclosed herein provides the thickening associated with a pH responsive microgel such as poly(acrylic) acid polymer in combination with good mechanical properties attributed to one or more water soluble or a blend of water soluble and water swellable thermoplastic polyurethanes (TPU). The reversible, noncovalent interactions of the thermoplastic urethane hard segments such as hydrogen bonding interactions hydrophobic association allows the gel to be reshaped and molded by external stimulus such as heat and or heat and pressure and then cooled to room temperature and be resistant to compression shear and strain.

In one embodiment, the technology disclosed herein provides a dual network hydrogel composition including a) a poly(acrylic) acid crosslinked polymer derived from one or more olefinically unsaturated polymerizable carboxylic monomers; and b) one or more thermoplastic polyurethane (TPU) polymers; wherein the polymer content is from about 2.0 wt % to about 8 wt % of the total composition.

The technology disclosed herein further provides a hydrogel in which the cross-linked polymer is a carbomer copolymer, a carbomer homopolymer, carbomer interpolymer, or a polycarbophil.

The technology disclosed herein further provides a hydrogel in which the poly(acrylic) acid polymer is cross-linked with an allyl ether cross-linking agent or divinyl glycol.

The technology disclosed herein further provides a hydrogel in which the allyl ether cross-linking agent comprises one or more of allyl pentaerythritol, allyl sucrose, or trimethpropanediolyl ether (TMPDE).

The technology disclosed herein further provides a hydrogel in which the TPU polymer comprises the reaction product of (i) at least one aliphatic or aromatic diisocyanate; (ii) a polyol component comprising at least one polyether polyol having a number average molecular weight of at least 300; and (iii) optionally, a chain extender component.

The technology disclosed herein further provides a hydrogel in which the chain extender comprises an aliphatic diol.

The technology disclosed herein further provides a hydrogel in which the polyether polyol comprises polyethylene glycol, polypropylene glycol or combinations thereof

The technology disclosed herein further provides a hydrogel in which wherein the polyether polyol component comprises a blend of polyethylene glycol polyols having number average molecular weights (Mn) of at least 300 and at least 1450.

The technology disclosed herein further provides a hydrogel in which the polyol component comprises a blend of polyethylene glycol polyols having number average molecular weights (Mn) of at least 1450 and at least 8000.

The technology disclosed herein further provides a hydrogel in which the polyol component comprises a blend of polyethylene glycol and polypropylene glycol.

The technology disclosed herein further provides a hydrogel in which the cross-linked polymer is partially neutralized.

The technology disclosed herein further includes a comonomer, the comonomer comprising one or more of at least one acrylic acid ester of the formula:

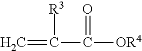

wherein R³ is hydrogen, methyl or ethyl and R⁴ is an alkyl group containing 1 to 30 carbon atoms, in an amount of less than 30 weight percent based upon the weight of the carboxylic acid or anhydride plus the acrylic acid ester.

The technology disclosed herein further provides a hydrogel in which the ratio of the components (a) to (b) is from 0.5:1 to 10:1.

The technology disclosed herein further provides a hydrogel in which the polymer content is from about 3.5 wt % to about 5 wt % of the total composition.

The technology disclosed herein further provides a hydrogel in which the polymer content is at least 2 wt %.

The technology disclosed herein further provides a hydrogel in which the poly(acrylic) acid cross-linked polymer is present in an amount of at least 0.5 wt % of the total composition.

The technology disclosed herein further provides a hydrogel in which the poly(acrylic) acid cross-linked polymer is present in an amount from 0.5 wt % to 3 wt % of the total composition.

The technology disclosed herein further provides a hydrogel further including one or more of a pharmaceutical, a biologically active compound, an absorptive material, a personal care compound, an active ingredient, a therapeutic aid, or combinations thereof.

The technology disclosed herein further provides a wound covering including the hydrogel.

The technology disclosed herein further provides a hydrogel in which the hydrogel is in sheet form.

The technology disclosed herein further provides a hydrogel in which the sheet has a thickness of from 0.2 to 0.7 cm.

The technology disclosed herein further provides a hydrogel in which the TPU polymer comprises a water soluble TPU or a blend of a water soluble and water swellable TPU.

The technology disclosed herein further provides a hydrogel in which the chain extender component comprises one or more of diethylene glycol or a C₃-C₁₂ diol and is present in an amount from 0.4 wt % to 4 wt %.

The technology disclosed herein further provides a dual network hydrogel composition including a) a crosslinked polymer derived from one or more olefinically unsaturated polymerizable carboxylic monomers; b) an optional comonomer; and c) a thermoplastic polyurethane (TPU) comprising the reaction product of i) an aliphatic or aromatic diisocyanate; and ii) a polyol component comprising of at least one polyethylene glycol having a number average molecular weight (Mn) of at least 1450 in which in the composition is thermoformable and semi-solid.

The technology disclosed herein further provides a hydrogel in which the hydrogel composition is thermoformable at temperatures of from about 50° C. to about 90° C.

The technology disclosed herein further provides a hydrogel in which the total polymer content is at least 2.0 wt % of the total composition.

The technology disclosed herein further provides a hydrogel in which the comonomer comprising one or more of at least one acrylic acid ester of the formula:

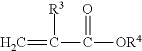

wherein R³ is hydrogen, methyl or ethyl and R⁴ is an alkyl group containing 1 to 30 carbon atoms, in an amount of less than 30 weight percent based upon the weight of the carboxylic acid or anhydride plus the acrylic acid ester.

The technology disclosed herein further provides a hydrogel in which the comonomer is present in an amount from 1 wt % to 65 wt %, or from 1 to 15 wt %.

The technology disclosed herein further provides a hydrogel in which the polyol component of the TPU polymer comprises a blend of polyethylene glycol polyols having number average molecular weights (Mn) of at least 1450 and at least 8000.

The technology disclosed herein further provides a dual network hydrogel composition including a) a homopolymer of a crosslinked polymer derived from one or more olefinically unsaturated polymerizable carboxylic monomers; and b)

a hydrophilic thermoplastic polyurethane polymer; in which the total polymer content of the composition is at least 2.0 wt % of the total composition.

The technology disclosed herein further provides a hydrogel in which the hydrophilic thermoplastic polyurethane composition includes (i) an aromatic diisocyanate component; (ii) at least one polyether polyol having a number average molecular weight of at least 300; and an optional chain extender component.

The technology disclosed herein further provides a hydrogel in which the polymer content is from 2.0 wt % to 8 wt % of the total polymer composition.

A article including a dual network hydrogel composition, the hydrogel composition including a) a poly(acrylic) acid crosslinked polymer derived from one or more olefinically unsaturated polymerizable carboxylic monomers; and b) one or more thermoplastic polyurethane (TPU) polymers; in which the polymer content is from about 2.0 wt % to about 8 wt % of the total composition.

The technology disclosed herein further provides a medical article in which the article includes one or more of a wound covering, a dressing, a controlled drug delivery device, a microfluidic device, or a biosensor.

The technology further provides a wound covering article including a backing and a facing.

The technology further provides a wound covering article in the form of a sheet, a gel or an impregnated gauze.

The technology disclosed herein further provides a medical article in which the article is a personal care article, a pharmaceutical article, or a health care article.

The technology disclosed herein further provides a dermal, mucosal or transdermal delivery agent for the delivery of chemically and physically active ingredients including a dual network hydrogel composition including a) a poly(acrylic) acid crosslinked polymer derived from one or more olefinically unsaturated polymerizable carboxylic monomers; and b) one or more thermoplastic polyurethane (TPU) polymers; wherein the polymer content is from about 2.0 wt % to about 8 wt % of the total composition.

The technology disclosed herein further provides a method of making a dual network hydrogel composition including the step of reacting a) a crosslinked polymer derived from one or more olefinically unsaturated polymerizable carboxylic monomers; and b) a thermoplastic polyurethane comprising the reaction product of i) a polyisocyanate; and ii) a polyol component comprising of at least one polyethylene glycol polyol having a molecular weight (Mn) of at least 800; and (iii) an optional chain extender; in which a) and b) are reacted at a ratio of 0.5:1 to 10:1.

The technology disclosed herein further provides a dual network hydrogel composition including a) a pH sensitive microgel cross-linked poly(acrylic) acid; and b) one or more water soluble or water swellable thermoplastic polyurethane polymers.

The technology disclosed herein further provides a hydrogel which exhibits a Yield Stress of from 1000 to 7500 Pa and has pH sensitive microgel content of 0.25 to 3 wt %, a water soluble TPU from 1.5 to 4.5 wt % and an ethanol/water soluble TPU of 0 to 1 wt %.

The technology disclosed herein further provides a hydrogel which includes a polymer content of at least 2.3 wt %, or at least 3 wt % of the total composition.

The technology disclosed herein further provides a hydrogel which includes a mixture of (i) a pH sensitive microgel polyacrylic acid and (ii) a water soluble polyether thermoplastic urethane or a blend of a water soluble thermoplastic urethane and a water swellable thermoplastic polyurethane and the hydrogel has a Yield Stress of at least 2,500 Pa.

The technology disclosed herein further provides a hydrogel in which the polymer content is at the most 8 wt %, or at most 7 wt % or at most 6 wt % of the total composition.

The technology disclosed herein further provides a hydrogel in which the water soluble or water swellable thermoplastic polyurethane polymers are the reaction product of i) a first water soluble polyether polyol having a molecular weight of at least 3000 daltons; (ii) a diisocyanate; and (iii) at least one of a second polyol having a molecular weight of up to 800 daltons, a third polyether polyol having a molecular weight of no more than 2500 daltons, or a chain extender.

The technology disclosed herein further provides a hydrogel in which the water soluble or water swellable thermoplastic polyurethane polymers are the reaction product of i) a first water soluble polyether polyol having a molecular weight of at least 3000 daltons; (ii) a diisocyanate; and (iii) at least two of a second polyol having a molecular weight of up to 800 daltons, a third polyether polyol having a molecular weight of no more than 2500 daltons, or a chain extender.

The technology disclosed herein further provides a method of forming a hydrogel wound dressing, or a dermal delivery hydrogel including reacting a) a crosslinked polyacrylic acid microgel; and b) a thermoplastic polyurethane polymer which is water soluble or water swellable which includes the reaction product of (i) a first water soluble polyether polyol having a molecular weight of at least 3000 daltons; (ii) a diisocyanate; and (iii) at least one of a second polyol having a molecular weight of up to 800 daltons, a third polyether polyol having a molecular weight of no more than 2500 daltons, or a chain extender.

The technology disclosed herein further provides a method of forming a hydrogel wound dressing or dermal delivery hydrogel the microgel and TPU together with water form at least 2.0 wt % total polymer.

The technology disclosed herein further provides a method further includes forming the hydrogel and including an active agent to form a hydrogel sheet which includes the active agent dispersed in the hydrogel.

The technology disclosed herein further provides a hydrogel wound dressing or a dermal delivery hydrogel which includes at least 92 wt. % water, 1 to 5 wt. % of a thermoplastic polyurethane polymer, 0.5 to 4 wt % pH sensitive microgel and at least one active agent, wherein the polymer content of the hydrogel is at least 2.0 wt % of the total composition.

The technology disclosed herein further provides a hydrogel composition in which the hard segment of the TPU comprises from 0.25 wt % to 6 wt %.

The technology disclosed herein further provides a hydrogel composition in which the hard segment of the TPU comprises at least 0.25 wt %, or at least 0.35 wt %.

The technology disclosed herein further provides a hydrogel composition in which the soft segment of the TPU comprises at least 80 wt %, or from 80 wt % to 95 wt %.

### BRIEF DESCRIPTION OF THE DRAWING FIGURES

FIG. 1 is a graph illustrating yield stress of the inventive polymer composition versus a comparative composition at the same ratio and concentration. Desired Yield Stress value is at least 2,500 Pa.

FIG. 2 is a graph illustrating the plot of G′ and G″ show a cross over point and Yield stress shows plateau of the shear stress vs. shear strain curve.

FIG. 3 is a graph illustrating the plot of G′ and G″ do not show a cross over point and Yield stress shows plateau of the shear stress vs. shear strain curve

### DETAILED DESCRIPTION OF THE INVENTION

Various preferred features and embodiments will be described below by way of non-limiting illustration.

The dual network hydrogel described herein is prepared from least two polymers, namely, a pH sensitive partially-neutralized, cross-linked microgel polymer and one or more water soluble thermoplastic polyurethane (TPU) to provide a semi-solid hydrogel which is thermoformable. By thermoformable it is meant that the polymer has increased flow and moldability at temperatures above room temperature yet does not move to a liquid state and resists flow and shape change at room temperatures.

The water soluble TPU disclosed herein has hard segments that can reversibly “microphase separate” to form periodic nanostructures at lower temperature in water. The hard segments act as thermally reversible crosslinks. In this hydrogel this nanostructure gives surprising Yield Stress to the hydrogel. This can be seen in the comparative examples of Polyethylene Oxide which does not have this microphase separation. This can be seen in the ability to heat the hydrogel to between 50° C. and 80° C. and extrude through a die to form a sheet. To compress a disparate mass between two plates with a cavity at 50 C and have the mass flow and meld to a single sheet of the thickness and shape of the cavity. Injection molding is another thermoprocess that is envisioned where at elevated temperature the hydrogel may be rammed or screw fed into a mould cavity which solidifies into a shape that has conformed to the contour of the mould.

Because of their nature, the hydrogels compositions of the invention are better tested using strain response characterization rather than methods such as kinematic (Brookfield) viscosity determination. The inventive hydrogels do not display liquid like behavior in their response to strain at room temperature. Using rheological analysis, the hydrogel viscoelastic behavior can be evaluated using a strain rheometer through small amplitude oscillations. The composition of the inventive hydrogel in respect to polymer composition and concentration control this behavior. The hydrogel composition can be related to material in use properties and general sensory performance.

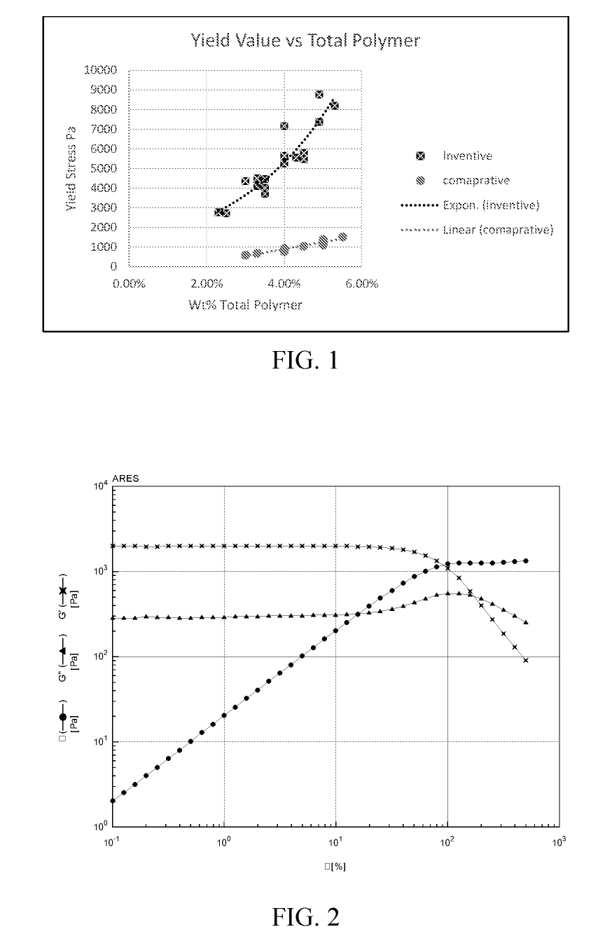

The key rheological test that shows the significant difference between the inventive system and comparative systems is the Yield Stress of the hydrogel. Yield Stress is the point at when increasing the applied strain, the hydrogel first starts to deform. The inventive hydrogel compositions do not deform and flow, but maintain their shape until high strain is reached. Comparative compositions flow and do not maintain their shape at similar polymer concentrations. The inventive compositions show Yield Stress above 2,500 Pa. FIG. 1 illustrates how the inventive gels have higher Yield Stress than the comparative gels at the same total solid concentrations.

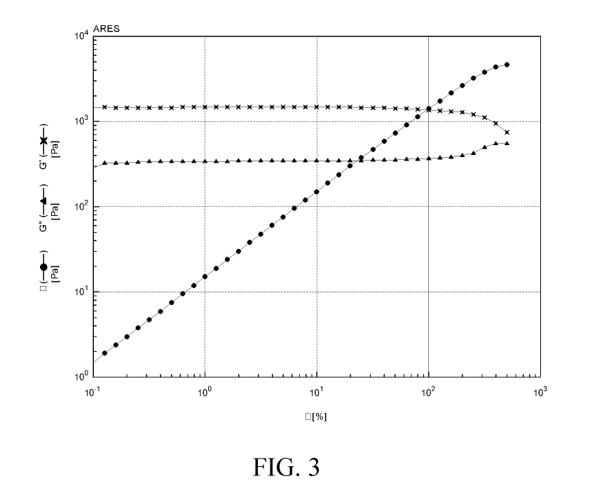

Yield Stress is defined as the value as measured with a strain rheometer is where the value of storage modulus (G′) crosses the loss modulus (G″) value as shown in FIG. 2. Yield Stress may also be defined as the value measured at the maximum value of the shear stress vs. shear strain curve if G′ does not cross G″, as illustrated in FIG. 3.

When the loss modulus G″—the energy dissipated—is larger than the storage modulus G′ at high frequency, the viscous behavior dominates and the sample displays liquid-like behavior. These compositions do not have a crossover point, [(Pa) against τ (Pa)] so do not display a liquid like state at higher % strain. The inventive hydrogels show good properties have G″ above 450 Pa at 100 Pa. The inventive hydrogels reach this value at lower total solids than the comparative systems.

Another rheological method to represent the properties of the hydrogel is tan δ which is G″/G′. Within the inventive hydrogels, tan δ is consistent and higher than the comparative hydrogels. With acceptable formulations (medium adhesion) have a tan δ value that lays above 0.180 and (between 0.180 and 0.300). Unacceptable formulations show low adhesion with a tan δ value lower than 0.180.

### Polyacrylic Acid

The term poly(acrylic) acid or acrylic acid polymer is used to encompass a variety of polymers having high percentages of polymerizable monomers therein with pendant carboxylic acid groups or anhydrides of polycarboxylic acid. These are described in more detail in U.S. Pat. Nos. 2,798,053; 3,915,921; 4,267,103; 5,288,814; and 5,349,030 hereby incorporated by reference. The term polyacrylic acid is used to include various homopolymers, copolymers, and interpolymers, wherein at least 50 or 75 mole percent of the repeating units have pendant carboxylic acid groups or anhydrides of dicarboxylic acid groups. While acrylic acid is the most common primary monomer used to form polyacrylic acid the term is not limited thereto but includes generally all α-β unsaturated monomers with carboxylic pendant groups or anhydrides of dicarboxylic acids as described in U.S. Pat. No. 5,349,030.

The carboxyl containing polymers are prepared from monomers containing at least one activated >C═C< group and carboxyl group. Such polymers are homopolymers of an unsaturated, polymerizable carboxylic monomers such as acrylic acid, methacrylic acid, maleic acid, itaconic acid, maleic anhydride, and the like, and copolymers of polymerizable carboxylic monomers with acrylate esters, acrylamides, olefins, vinyl esters, vinyl ethers, or styrenics. The carboxyl containing polymers have molecular weights greater than about 500 to as high as several million, usually greater than about 10,000 to 900,000 or more.

Copolymers, for example, include copolymers of acrylic acid with small amounts of polyalkenyl polyether cross-linkers that are gel-like polymers, which, especially in the form of their salts, absorb large quantities of water or solvents with subsequent substantial increase in volume. Other useful carboxyl containing polymers are described in U.S. Pat. No. 3,940,351, directed to polymers of unsaturated carboxylic acid and at least one alkyl acrylic or methacrylic ester where the alkyl group contains 10 to 30 carbon atoms, and U.S. Pat. Nos. 5,034,486; 5,034,487; and 5,034,488; which are directed to maleic anhydride copolymers with vinyl ethers. Other types of such copolymers are described in U.S. Pat. No. 4,062,817 wherein the polymers described in U.S. Pat. No. 3,940,351 contain additionally another alkyl acrylic or methacrylic ester and the alkyl groups contain 1 to 8 carbon atoms. Carboxylic polymers and copolymers such as those of acrylic acid and methacrylic acid also may be cross-linked with polyfunctional materials as divinyl benzene, unsaturated diesters and the like, as is disclosed in U.S. Pat. Nos. 2,340,110; 2,340,111; and 2,533,635. The disclosures of all of these U.S. patents are hereby incorporated herein by reference.

The carboxylic monomers are the olefinically-unsaturated carboxylic acids containing at least one activated carbon-to-carbon olefinic double bond, and at least one carboxyl group; that is, an acid or function readily converted to an acid containing an olefinic double bond which readily functions in polymerization because of its presence in the monomer molecule, either in the alpha-beta position with respect to a carboxyl group, —C═C—COOH; or as part of a terminal methylene grouping, CH₂═C<. Olefinically-unsaturated acids of this class include such materials as the acrylic acids typified by the acrylic acid itself, alpha-cyano acrylic acid, beta methylacrylic acid (crotonic acid), alpha-phenyl acrylic acid, beta-acryloxy propionic acid, cinnamic acid, p-chloro cinnamic acid, 1-carboxy-4-phenyl butadiene-1,3, itaconic acid, citraconic acid, mesaconic acid, glutaconic acid, aconitic acid, maleic acid, fumaric acid, and tricarboxy ethylene. As used herein, the term “carboxylic acid” includes the polycarboxylic acids and those acid anhydrides, such as maleic anhydride, wherein the anhydride group is formed by the elimination of one molecule of water from two carboxyl groups located on the same carboxylic acid molecule. Maleic anhydride and other acid anhydrides useful herein have the general structure

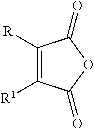

wherein R and R′ are selected from the group consisting of hydrogen, halogen and cyanogen (—C≡N) groups and alkyl, aryl, alkaryl, aralkyl, and cycloalkyl groups such as methyl, ethyl, propyl, octyl, decyl, phenyl, tolyl, xylyl, benzyl, cyclohexyl, and the like.

The preferred carboxylic monomers are the monoolefinic acrylic acids having the general structure:

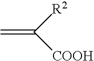

wherein R² is a substituent selected from the class consisting of hydrogen, halogen, and the cyanogen (—C≡N) groups, monovalent alkyl radicals, monovalent aryl radicals, monovalent aralkyl radicals, monovalent alkaryl radicals and monovalent cycloaliphatic radicals. Of this class, acrylic and methacrylic acid are most preferred. Other useful carboxylic monomers are maleic acid and its anhydride.

The polymers include both homopolymers of carboxylic acids or anhydrides thereof, or the defined carboxylic acids copolymerized with one or more other vinylidene monomers containing at least one terminal >C═CH₂ group. The other vinylidene monomers are present in an amount of less than 30 weight percent based upon the weight of the carboxylic acid or anhydride plus the vinylidene monomer(s). Such monomers include, for example, acrylate ester monomers including those acrylic acid ester monomers such as derivatives of an acrylic acid represented by the formula

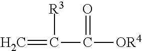

wherein R⁴ is an alkyl group having from 1 to 30 carbon atoms, preferably 1 to 20 carbon atoms and R³ is hydrogen, methyl or ethyl, present in the copolymer in amount, for example, from about 1 to 40 weight percent or more. Representative acrylates include methyl acrylate, ethyl acrylate, propyl acrylate, isopropyl acrylate, butyl acrylate, isobutyl acrylate, methyl methacrylate, methyl ethacrylate, ethyl methacrylate, octyl acrylate, heptyl acrylate, octyl methacrylate, isopropyl methacrylate, 2-ethylhexyl methacrylate, nonyl acrylate, hexyl acrylate, n-hexyl methacrylate, and the like. Higher alkyl acrylic esters are decyl acrylate, isodecyl methacrylate, lauryl acrylate, stearyl acrylate, behenyl acrylate and melissyl acrylate. Mixtures of two or three or more long chain acrylic esters may be successfully polymerized with one of the carboxylic monomers. Other comonomers include olefins, including alpha olefins, vinyl ethers, vinyl esters, and mixtures thereof.

The polymers also may be cross-linked with any polyene, e.g. decadiene or trivinyl cyclohexane; acrylamides, such as methylene bis acrylamide; polyfunctional acrylates, such as trimethylol propane triacrylate; or polyfunctional vinylidene monomer containing at least 2 terminal CH₂═C< groups, including for example, butadiene, isoprene, divinyl benzene, divinyl naphthlene, allyl acrylates and the like. Particularly useful cross-linking monomers for use in preparing the copolymers are polyalkenyl polyethers having more than one alkenyl ether grouping per molecule. The most useful possess alkenyl groups in which an olefinic double bond is present attached to a terminal methylene grouping, CH₂═C<. They are made by the etherification of a polyhydric alcohol containing at least 2 carbon atoms and at least 2 hydroxyl groups. Compounds of this class may be produced by reacting an alkenyl halide, such as allyl chloride or allyl bromide, with a strongly alkaline aqueous solution of one or more polyhydric alcohols. The product may be a complex mixture of polyethers with varying numbers of ether groups. Analysis reveals the average number of ether groupings on each molecule. Efficiency of the polyether cross-linking agent increases with the number of potentially polymerizable groups on the molecule. It is preferred to utilize polyethers containing an average of two or more alkenyl ether groupings per molecule. Other cross-linking monomers include for example, diallyl esters, dimethallyl ethers, allyl or methallyl acrylates and acrylamides, tetraallyl tin, tetravinyl silane, polyalkenyl methanes, diacrylates, and dimethacrylates, divinyl compounds such as divinyl benzene, divinyl glycol, polyallyl phosphate, diallyloxy compounds and phosphite esters and the like. Typical agents are allyl pentaerythritol, allyl sucrose, trimethylolpropane triacrylate, 1,6-hexanediol diacrylate, trimethylolpropane diallyl ether, pentaerythritol triacrylate, tetramethylene dimethacrylate, ethylene diacrylate, ethylene dimethacrylate, triethylene glycol dimethacrylate, and the like. Allyl pentaerythritol, trimethylolpropane diallylether and allyl sucrose provide excellent polymers. When the cross-linking agent is present, the polymeric mixtures usually contain up to about 5% or less by weight of cross-linking monomer based on the total of carboxylic acid monomer, plus other monomers, if present, and more preferably about 0.01 to 3.0 weight percent.

Other vinylidene monomers may also be used, including the acrylic nitriles. The useful α,β-olefinically unsaturated nitriles are preferably the monoolefinically unsaturated nitriles having from 3 to 10 carbon atoms such as acrylonitrile, methacrylonitrile, and the like. Most preferred are acrylonitrile and methacrylonitrile. The amounts used are, for example, for some polymers are from about 1 to 30 weight percent of the total monomers copolymerized. Acrylic amides containing from 3 to 35 carbon atoms including monoolefinically unsaturated amides also may be used. Representative amides include acrylamide, methacrylamide, N-t-butyl acrylamide, N-cyclohexyl acrylamide, higher alkyl amides, where the alkyl group on the nitrogen contains from 8 to 32 carbon atoms, acrylic amides including N-alkylol amides of alpha, beta-olefinically unsaturated carboxylic acids including those having from 4 to 10 carbon atoms such as N-methylol acrylamide, N-propanol acrylamide, N-methylol methacrylamide, N-methylol maleimide, N-methylol maleamic acid esters, N-methylol-p-vinyl benzamide, and the like. Still further useful materials are alpha-olefins containing from 2 to 18 carbon atoms, more preferably from 2 to 8 carbon atoms; dienes containing from 4 to 10 carbon atoms; vinyl esters and allyl esters such as vinyl acetate; vinyl aromatics such as styrene, methyl styrene and chlorostyrene; vinyl and allyl ethers and ketones such as vinyl methyl ether and methyl vinyl ketone; chloroacrylates; cyanoalkyl acrylates such as α-cyanomethyl acrylate, and the α-, β-, and γ-cyanopropyl acrylates; alkoxyacrylates such as methoxy ethyl acrylate; haloacrylates as chloroethyl acrylate; vinyl halides and vinyl chloride, vinylidene chloride and the like; divinyls, diacrylates and other polyfunctional monomers such as divinyl ether, diethylene glycol diacrylate, ethylene glycol dimethacrylate, ethylene-bisacrylamide, allylpentaerythritol, and the like; and bis (β-haloalkyl) alkenyl phosphonates such as bis(β-chloroethyl) vinyl phosphonate and the like as are known to those skilled in the art. Copolymers wherein the carboxy containing monomer is a minor constituent, and the other vinylidene monomers present as major components are readily prepared in accordance with the process of this invention.

The steric stabilizer functions to provide a steric barrier which repulses approaching particles. A requirement for the steric stabilizer is that a segment of the dispersant (i.e., a hydrophobe) be very soluble in the solvent (the continuous phase in a nonaqueous dispersion polymerization process) and that another segment (i.e., a hydrophile) be at least strongly adhered to the growing polymer particle. Thus, the steric stabilizers of the present invention have a hydrophilic group and a hydrophobic group. The steric stabilizers are block copolymers comprising a soluble block and an anchor block having a molecular weight (i.e., chain length) usually well above 1000, but a hydrophobe length of more than 50 Angstroms, as calculated by the Law of Cosines. These dimensions are determined on the extended configuration using literature values for bond lengths and angles. Thus the steric stabilizers of the present invention are distinguishable from the prior art steric surfactants which may be block copolymers, but have hydrophobe lengths of less than 50 Angstroms. The steric stabilizer of the present invention has either a linear block or a comb configuration, and has a hydrophobe of sufficient length to provide a sufficient steric barrier.

When the steric stabilizer is a linear block copolymeric steric stabilizer, it is defined by the following formula:

C_{w}B-A-B_{y}ₓD_{z}

where A is a hydrophilic moiety, having a solubility in water at 25° C. of 1% or greater, a molecular weight of from about 200 to about 50,000, and selected to be covalently bonded to the B blocks;

B is a hydrophobic moiety, having a molecular weight of from about 300 to about 60,000, a solubility of less than 1% in water at 25° C., capable of being covalently bonded to the A blocks;

and D are terminating groups which can be A or B; can be the same or different groups, and will depend upon the manufacturing process since they are present to control the polymer length, to add other functionality, or as a result of the manufacturing process;

w is 0 or 1;
x is an integer of 1 or more,
y is 0 or 1, and
z is 0 or 1.

Examples of hydrophilic groups are polyethylene oxide, poly(1,3-dioxolane), copolymers of polyethylene oxide or poly(1,3-dioxolane), poly(2-methyl-2-oxazoline polyglycidyl trimethyl ammonium chloride, polymethylene oxide, and the like, with polyethylene oxide being preferred. Examples of hydrophobic groups are polyesters, such as those derived from 2-hydroxybutyric acid, 3-hydroxybutyric acid, 4-hydroxybutyric acid, 2-hydroxycaproic acid, 10-hydroxydecanoic acid, 12-hydroxydodecanoic acid, 16-hydroxyhexadecanoic acid, 2-hydroxyisobutyric acid, 2-(4-hydroxyphenoxy) propionic acid, 4-hydroxyphenylpyruvic acid, 12-hydroxystearic acid, 2-hydroxyvaleric acid, polylactones, such as caprolactone, butyrolactone, polylactams, such as those derived from caprolactam, polyurethanes, polyisobutylene, where the hydrophobe should provide a steric barrier of greater than 50 Angstroms, preferably greater than 75 Angstroms, with greater than 100 Angstroms being also preferred, and the like, with polyhydroxy fatty acids, such as poly(12-hydroxystearic acid) being preferred. The steric barrier is the length of the hydrophobe in its fully-extended condition. Such steric stabilizers are commercially available under the brand name Hypermer® from Croda.

Steric stabilizer molecules comprise both hydrophilic and hydrophobic units. Hydrophobic polymer units or hydrophobic blocks may be prepared by a number of well-known methods. These methods include condensation reactions of hydroxy acids, condensation of polyols (preferably diols) with polycarboxylic acids (preferably diacids). Other useful methods include polymerization of lactones and lactams, and reactions of polyols with polyisocyanates. Hydrophobic blocks or polymer units can be reacted with hydrophilic units by such reactions as are known to those skilled in the art. These reactions include condensation reactions and coupling reactions, for example. Subsequent to the steric stabilizer preparation, the stabilizers may be further reacted with modifying agents to enhance their utility. U.S. Pat. No. 4,203,877 to Alan S. Baker teaches making such steric stabilizers, and the entire disclosure thereof is incorporated herein by reference.

When the steric stabilizer is a random copolymeric comb steric stabilizer, it is defined by the following formula:

R⁵—(Z)m-(Q)n-R⁶,

where R⁵ and R⁶ are terminating groups and may be the same or different and will be different from Z and Q,
Z is a hydrophobic moiety having a solubility of less than 1% in water at 25° C.,
Q is a hydrophilic moiety, having a solubility of more than 1% in water at 25° C.,
m and n are integers of 1 or more, and are selected such that the molecular weight of the polymer is from about 100 to about 250,000.

Examples of the hydrophobic monomer unit or moiety are dimethyl siloxane, diphenyl siloxane, methylphenyl siloxane, alkyl acrylate, alkyl methacrylate, and the like, with dimethyl siloxane being preferred.

Examples of the hydrophilic monomer unit or moiety are methyl-3-polyethoxypropyl siloxane-Q-phosphate or sulfate, and the alkali metal or ammonium salts derived therefrom; units derived from polyethoxy (meth) acrylate containing from 1 to 40 moles of ethylene oxide; acrylic acid; acrylamide; methacrylic acid, maleic anhydride; dimethyl amino ethyl (meth)acrylate; or its salts with methyl chloride or dimethyl sulfate; dimethyl amino propyl(meth)acrylamide and its salts with methyl chloride or dimethyl sulfate, and the like, with methyl-3-polyethoxypropyl siloxane-Q-phosphate being preferred.

Examples of terminating agents are monohalo silanes, mercaptans, haloalkanes, alkyl aromatics, alcohols, and the like, which will produce terminating groups such as trialkyl silyl, alkyl, aryl alkyl, alcoholate, and the like, with the preferred terminating groups being trimethyl silyl.

Specific types of cross-linked polyacrylic acids include Carbopol® 981NF; Carbopol® 980NF; Pemulen TR1; Pemulen TR2; and carbomer interpolymer ETD-2020-NF; Ultrez 10NF, copolymers of acrylic acid and alkyl acrylates; copolymers of acrylic acid and alkyl vinyl ethers; and copolymers of ethylene and maleic anhydride. An approved polyacrylic acid for pharmaceutical applications are carbomer homopolymers, carbomer copolymers, carbomer interpolymers or polycarbophil, as described in the carbomer and polycarbophil compendia monographs in the U.S.

### Thermoplastic Polyurethane

The TPU of the hydrogel composition disclosed herein is formed from the reaction product of (i) a polyisocyanate component; (ii) a polyol component; and (iii) a chain extender component.

The TPU compositions described herein are made using a) a polyisocyanate component. The polyisocyanate and/or polyisocyanate component includes one or more polyisocyanates. In some embodiments, the polyisocyanate component includes one or more diisocyanates.

In some embodiments, the polyisocyanate and/or polyisocyanate component includes an α, ω-alkylene diisocyanate having from 5 to 20 carbon atoms.

Suitable polyisocyanates include aromatic diisocyanates, aliphatic diisocyanates, or combinations thereof. In some embodiments, the polyisocyanate component includes one or more aromatic diisocyanates. In some embodiments, the polyisocyanate component is essentially free of, or even completely free of, aliphatic diisocyanates. In other embodiments, the polyisocyanate component includes one or more aliphatic diisocyanates. In some embodiments, the polyisocyanate component is essentially free of, or even completely free of, aromatic diisocyanates.

Examples of useful polyisocyanates include aromatic diisocyanates such as 4,4′-methylenebis(phenyl isocyanate) (MDI), m-xylene diisocyanate (XDI), phenylene-1,4-diisocyanate, naphthalene-1,5-diisocyanate, and toluene diisocyanate (TDI); as well as aliphatic diisocyanates such as isophorone diisocyanate (IPDI), 1,4-cyclohexyl diisocyanate (CHDI), decane-1,10-diisocyanate, lysine diisocyanate (LDI), 1,4-butane diisocyanate (BDI), hexane-1,6-diisocyanate (HDI), 3,3′-dimethyl-4,4′-biphenylene diisocyanate (TODI), 1,5-naphthalene diisocyanate (NDI), and dicyclohexylmethane-4,4′-diisocyanate (H12MDI). Mixtures of two or more polyisocyanates may be used. In some embodiments, the polyisocyanate is MDI and/or H12MDI. In some embodiments, the polyisocyanate includes MDI. In some embodiments, the polyisocyanate includes H12MDI.

In some embodiments, the thermoplastic polyurethane is prepared with a polyisocyanate component that includes H12MDI. In some embodiments, the thermoplastic polyurethane is prepared with a polyisocyanate component that consists essentially of H12MDI. In some embodiments, the thermoplastic polyurethane is prepared with a polyisocyanate component that consists of H12MDI.

In some embodiments, the thermoplastic polyurethane is prepared with a polyisocyanate component that includes (or consists essentially of, or even consists of) H12MDI and at least one of MDI, HDI, TDI, IPDI, LDI, BDI, PDI, CHDI, TODI, and NDI.

In some embodiments, the polyisocyanate used to prepare the TPU and/or TPU compositions described herein is at least 50%, on a weight basis, a cycloaliphatic diisocyanate. In some embodiments, the polyisocyanate includes an α, ω-alkylene diisocyanate having from 5 to 20 carbon atoms.

In some embodiments, the polyisocyanate used to prepare the TPU and/or TPU compositions described herein includes hexamethylene-1,6-diisocyanate, 1,12-dodecane diisocyanate, 2,2,4-trimethyl-hexamethylene diisocyanate, 2,4,4-trimethyl-hexamethyl ene diisocyanate, 2-methyl-1,5-pentamethylene diisocyanate, or combinations thereof.

### The Polyol Component

The TPU compositions described herein are made using b) a polyol component. Polyols include polyether polyols.

Suitable polyols, which may also be described as hydroxyl terminated intermediates, when present, may include one or more hydroxyl terminated polyethers, polyether/polyester blocks, or mixtures thereof.

Suitable hydroxyl terminated polyether intermediates include polyether polyols. In one embodiment, the water soluble thermoplastic polyurethane (denoted TPU(1)) includes a (water soluble) soft segment which is derived from a first high molecular weight polyether polyol (Polyol A). Polyol A may be of the general form HO—(R¹(R²)O)ₙ—H, where:

R¹ is selected from C₂-C₄ alkylene groups and mixtures thereof, such as —CH₂CH— and —CH₂CH₂CH—,

R² is a side group and is selected from H and C₁-C₂ alkyl groups and mixtures thereof, and

n is an integer which represents the average number of ether units by weight in each polyol, and where n is at least 20 (Molecular weight of 2000).

Useful commercial polyether polyols include poly(ethylene glycol) comprising ethylene oxide reacted with ethylene glycol, poly(propylene glycol) comprising propylene oxide reacted with propylene glycol. Suitable polyether polyols also include polyamide adducts of an alkylene oxide and can include, for example, ethylenediamine adduct comprising the reaction product of ethylenediamine and propylene oxide, diethylenetriamine adduct comprising the reaction product of diethylenetriamine with propylene oxide, and similar polyamide type polyether polyols.

Copolyethers can also be utilized in the described compositions. Typical copolyethers include the reaction product of THF and ethylene oxide or THF and propylene oxide. These are available from BASF as PolyTHF® B, a block copolymer, and PolyTHF® R, a random copolymer.

The various polyether intermediates polylol generally have a number average molecular weight (Mn) as determined by assay of the terminal functional groups which is an average molecular weight greater than about 1450, such as from about 1,450 to about 12,000, or from about 2000 to about 10,000, or from about 1,450 to about 8,000. In one embodiment, they include a PEG having an Mn of 8000. In some embodiments, the polyether intermediate includes a blend of two or more different molecular weight polyethers, such as a blend of 300 Mn and 1,450 Mn PEG or a blend of 1450 Mn and 8,000 Mn PEG, or 300 and 8000 Mn.

The polyol component, when present, may include poly(ethylene glycol), poly(trimethylene oxide), ethylene oxide capped poly(propylene glycol), poly(hexamethylene carbonate) glycol, poly(pentamethylene carbonate) glycol, poly(trimethylene carbonate) glycol, dimer fatty acid based polyester polyols, or any combination thereof.

In some embodiments, the polyol component includes a polyether polyol. In some embodiments, the polyol component is essentially free of or even completely free of polyester polyols.

In some embodiments, the polyol component includes ethylene oxide, propylene oxide, butylene oxide, styrene oxide, poly(propylene glycol), poly(ethylene glycol), copolymers of poly(ethylene glycol) and poly(propylene glycol), epichlorohydrin, and the like, or combinations thereof. In some embodiments the polyol component includes poly(ethylene glycol).

The polyol component, in some embodiments, may include a multi-block polyol. The multi-block polyol can include combinations of polyether with polyester, for example, polyethylene oxide polyether (PEO)-polycaprolactone (PCL)) or (PCL-PEO-PCL) which give good control over hydrophilicity, degradation and mechanical properties. The use of multiblock polyether products PEO-PPO (polypropylene oxide—PEO better known as Pluronics® (a registered trademark of BASF Corporation) and block polyester such as PCL-PEO-PPO-PEO-PCL may also be used. It is also contemplated that alternative ester and ether blocks may be used, for example, multiblock polyethers in combination with a block polyester.

In some embodiments, the water soluble polyol segment may be a combination of a water soluble high molecular weight polyol, for example a molecular weight of 8000, and a medium molecular weight polyol (hydrophilic or hydrophobic) and a low molecular weight polyol to create soft segment and intermediate segment and hard segment polyurethane. Thus, in one embodiment, the TPU may have a soft segment content of at least 80 wt %, or from 80 wt % to 95 wt %, or about 90 wt %, or from 84 wt % to 92 wt %; an intermediate segment of at least 3.0 wt %, or from 3.0 wt % to 12 wt %, or from 3.0 wt % to 10.5 wt %; a soft and intermediate segment content of at least 93 wt %; and a hard segment content of at least 0.25 wt %, or from 0.25 wt % to 6 wt %;

### The Optional Chain Extender Component

The TPU compositions described herein can further include c) a chain extender component. Chain extenders include diols, diamines, and combination thereof. Chain extenders include diols, diamines, and combinations thereof. The chain extender may have a molecular weight of up to 500 daltons or up to 300 daltons, such as at least 46 daltons.

One or more short chain polyols having from 2 to 20, or 2 to 12, or 2 to 10 or 2-8 carbon atoms may be used as chain extenders in the polyurethane forming composition to increase the molecular weight of the polyurethane. Examples of chain extenders include lower aliphatic polyols and short chain aromatic glycols having molecular weights of less than 500 or less than 300. Suitable chain extenders include organic diols (including glycols) having a total of from 2 to about 20 carbon atoms such as alkane diols, cycloaliphatic diols, alkylaryl diols, and the like. Exemplary alkane diols include ethylene glycol, diethylene glycol, 1,3-propanediol, 1,3-butanediol, 1,4-butanediol, (BDO), 1,3-butanediol, 1,5-pentanediol, 2,2-dimethyl-1,3-propanediol, propylene glycol, dipropylene glycol, 1,6-hexanediol, 1,7-heptanediol, 1,9-nonanediol, 1,10-decanediol, 1,12-dodecanediol, tripropylene glycol, triethylene glycol, and 3-methyl-1,5-pentanediol. Examples of suitable cycloaliphatic diols include 1,2-cyclopentanediol, and 1,4-cyclohexanedimethanol (CHDM). Examples of suitable aryl and alkylaryl diols include hydroquinone di(β-hydroxyethyl)ether (HQEE), 1,2-dihydroxybenzene, 1,3-dihydroxybenzene, 1,4-dihydroxybenzene, 1,2,3-trihydroxybenzene, 1,2-di(hydroxymethyl)benzene, 1,4-di(hydroxymethyl)benzene, 1,3-di(2-hydroxyethyl)benzene, 1,2-di(2-hydroxyethoxy)benzene, 1,4-di(2-hydroxyethoxy)benzene, bisethoxy biphenol, 2,2-di(4-hydroxyphenyl)propane (i.e., bisphenol A), bisphenol A ethoxylates, bisphenol F ethoxylates, 4,4-isopropylidenediphenol, 2,2-di[4-(2-hydroxyethoxy)phenyl]propane (HEPP), and mixtures thereof and the like.

Mixtures of one or more of the above chain extenders can also be utilized. When present, the chain extender component can be utilized in an amount from 0.4 wt % to about 4.0 wt %.

Chain extenders with functionality greater than 2 may be used so long as the resulting TPU retains its thermoplasticity. Examples of such chain extenders include trimethylolpropane (TMP), glycerin and pentaerythritol. Generally, the addition of such chain extenders should not exceed 10% relative to the weight of the difunctional chain extenders.

In one embodiment, the chain extender is selected from 1,4-butanediol, and 1,10-decanediol.

Chain extenders can also be based on diamines. Exemplary diamines may have molecular weights of less than 500, and include, for example, as ethyl enediamine, diethylenediamine, tetramethylenediamine, hexamethylenediamine, diethylenetriamine triethylenetetramine, tetraethylenepentamine, pentaethylenehexamine, piperazine, morpholine, substituted morpholine, piperidine, substituted piperidine, 2-amino-1-ethylpiperazine hydrazine, 1,4-cyclohexanediamine, and mixtures thereof. Alkanolamines, such as ethanolamine, diethanolamine, and triethanolamine, may also be used. Further examples of chain lengthening agents include aniline, and the like.

The molar amount or ratio of the total hydroxyl groups of the one or more chain extenders utilized to the total hydroxyl groups of Polyols A, B, and C may be from about 0.1 to about 5.0, or from about 0.2 to about 4.0, or from about 0.4 to about 2.5.

In some embodiments, a blend of a water soluble thermoplastic polyurethane and water swellable thermoplastic polyurethane where the water swellable thermoplastic polyurethane is soluble in a mixture of an organic solvent miscible in water and water, such as alcohol and water, may be used. As an alcohol, ethanol and isopropanol may be used. As an alternative to alcohol, tetrahydrofuran, dimethylacetamide, dimethylformamide, or other water-miscible non-aqueous solvents can be used.

### Additional TPU Components

In some embodiments, the TPU described herein will further include an optional chain terminating agent. Chain terminating agents are well known and may be a monohydroxyl or mono primary amine or any other mono function compound that reacts with a di-isocyanate to terminate the step growth polymerization at the end of the polymerchain. These may be the same or different on either end of the polymer. The chain terminating agent may have a number average molecular weight ranging from 100 to 8000, linked to the polymer via a urethane or urea bond.

Examples of chain terminating agents include mono amine- or mono alcohol-terminated polyalkylene oxides, silicones, alkyl, alkylesters, polyalkylene esters and mixtures thereof. In some embodiments, a chain terminating group that may be used in the polyurethane copolymers according to the present invention include monofunctional polyethylene oxides, monofunctional polytetramethylene oxides, monofunctional polypropylene oxides, monofunctional siloxanes, and mixtures and/or copolymers thereof. Dodecylamines, alkoxylated alcohols such as cetereth-20, steareth 20 and the like. In one embodiment, the amount of chain terminating agent is from 0 wt %-2 weight % based on the total weight of the dry polyurethane copolymer.

The hydrogel composition described herein may be formed by any of several methods. In one embodiment, the hydrogel composition is formed by forming a crosslinked poly(acrylic) acid polymer gel and adding a fine powder of one or more TPU(s). In one embodiment, materials are added to water in the percentages shown in the following table, to give a 100 parts total.

TABLE 1

| Component | % Min | % Max |
|---|---|---|
| Carbopol | 0.3 | 3 |
| TPU (1) | 1.5 | 4.5 |
| TPU (2) | 0 | 1.5 |
| Total polymer | 2.3 | 6.0 |

In one embodiment, the total polymer content of the hydrogel composition can be from about 2.0 wt % to about 8 wt %, and in another embodiment from about 2.3 wt % to about 7 wt %, or at least 2.0 wt %, or at least 2.3 wt %, or not greater than 8 wt %. In some embodiments, the Yield Stress of they hydrogel composition may be at least 2,500 Pascals in water (where water may be at least 95% water and 5% water miscible solvent.)

The TPU powder may be obtained by cyrogrinding, electrospraying, spray drying, or any other means as known to one skilled in the art to reduce the particle size of the TPU. In one embodiment, the TPU particle size may be less than or equal to 400 microns.

It has been found that the degree of neutralization of the poly(acrylic) acid polymer has a direct impact on preparation of the blended poly(acrylic) acid polymer and TPU as well as the final hydrogel properties. Accordingly, in one embodiment, prior to blending of the two polymers, the poly(acrylic) acid polymer is partially neutralized from an initial pH of from about 2.0 up to about 8.0 or from about 2.0 up to about 6.5 or from about 2.0 up to about 4.0. In one embodiment the amount of neutralizer used is from 25% to 50% of the theoretical value necessary to achieve a polymer solution of pH 7. In another embodiment, the amount of neutralization is from 10% to 75% of the acid content of the polymer. In a still further embodiment, the pH of the polymer solution is from 4 to 8. Neutralization can be carried out with any convenient neutralizing agent or compound such as ammonium hydroxide, sodium hydroxide, other alkali hydroxides, borates, phosphates, pyrophosphates or polyphosphates; an amino acid, such as arginine; AMP-95 (2-Amino-2-Methyl-1-Propanol) a product of Angus Chemical, cocamine, oleamine, diisopropanolamine, diisopropylamine, dodecylamine, PEG-15 cocoamine, morpholine, tetrakis(hydroxypropyl)ethylenediamine, triamylamine, triethanolamine, triethylamine, or tromethamine (2-Amino 2-Hydroxymethyl-1,3-propanediol). In some embodiments, neutralizing agents include NaOH, tetrakis(hydroxypropyl)ethylenediamine, triethanolamine, and tromethamine.

The poly(acrylic) acid polymer and hydrophilic TPU are then blended together. In one embodiment, a TPU powder can be added to a poly(acryic) acid polymer solution. In another embodiment, TPU may be dissolved in water and added to a partially neutralized poly(acrylic) acid polymer. In another embodiment, an acid dispersion of poly(acrylic) acid polymer is added to a high pH TPU. In one embodiment, the ratio, by weight, of hydrophilic TPU to poly(acrylic) acid polymer is from 0.5:1 to 10:1, and in another embodiment from 0.6:1 to 8:1. Optionally, additional water or other solvent such as alcohols, polyols, or polyalkoxides can be added. Such additional water or solvent is dependent upon the desired final qualities and physical constraints of individual formulations.

In some embodiments, the hydrogel composition described herein may be thermoformed, where thermoformed is meant to define the property of having increased flow and moldability at temperatures above room temperature yet not moving to a liquid state and resisting flow and shape change at room temperatures. Thermoplastic is known to those skilled in the art as a material that is processable as a melt at elevated temperature. The water soluble thermoplastic polyurethane hard segments of this invention can reversibly “microphase separate” to form periodic nanostructures at lower temperature in the water. The hard segments act as thermally reversible crosslinks. In this hydrogel this nanostructure gives surprising Yield Stress to the hydrogel. This can be seen in the comparative examples of Polyethylene Oxide which does not have this microphase separation. This can be seen in the ability to heat the hydrogel to between 50 and 80 C and extrude through a die to form a sheet. To press a disparate mass between two plates with a cavity at 50 C and have the mass flow and meld to a single sheet of the thickness of the cavity. Injection molding is another thermoprocess that is envisions where at elevated temperature the hydrogel may be rammed or screw fed into a mould cavity which solidifies into a shape that has conformed to the contour of the mould.

In some embodiments, the hydrogels disclosed herein may be sterilized. Sterilization is the treatment process that rids materials of possible contaminants, including microbial life, bacteria, fungi and viruses. In order to limit transmission of these contaminants, the medical industry requires certain levels of sterilization. Several sterilization methods may be used. In one embodiment, sterilization may be conducted by immersing the product in ethylene oxide gas in a chamber, then aerating it. In another embodiment, the product is put in a sterilization chamber that is vacuumed and filled with hydrogen peroxide vapor and then aerated. Sterilization involving ionizing energy that has low penetration and uses a high dose rate to eliminate contaminants may be used. An accelerator produces a beam of electrons that are focused on the product to be sterilized. Sterilization using an isotope source, usually Cobalt-60, to produce ionizing energy that flows through the product may also be used. This energy causes cellular damage to the organisms, ridding the product of them. Sterilization utilizing hot air, conducting heat through the equipment may be used. Objects are heated to a steady temperature and held for a certain length of time, depending on the material. Dry heat sterilization is very effective, as it can reach all surfaces of an assembled product.

### Active Agents

The hydrated polyurethane film includes one or more active agents. One or more excipients may also be present. One or more of the active agents and/or excipients may be introduced to the preformed dry film in a hydrating composition, such as water, an alcohol or other organic solvent, combination thereof, or the like. In another embodiment, one or more of the active agents and/or excipients is combined with the polyurethane polymer to form a casting solution and cast together to form a polyurethane polymer film, which may be then dried to form the dry polyurethane film.

Active agents useful herein may be categorized or described herein by their therapeutic and/or cosmetic benefit or their postulated mode of action or function. However, it is to be understood that the active and other ingredients useful herein can, in some instances, provide more than one cosmetic and/or therapeutic benefit or function or operate via more than one mode of action. Therefore, classifications herein are made for the sake of convenience and are not intended to limit an ingredient to the particularly stated application or applications listed.

Active agents useful herein may be delivered to the surface of the skin, known as the stratum corneum, may be delivered to the underlying portions of the skin known as the dermis and epidermis. Active agents may also be medicinal drug substances which penetrate through the initial layers of the skin to the underlying tissue, in this respect the active agents may have local effect and are not systemic. Active agents may also have percutaneous absorption and have a systemic effect where the active agent is considered a medicinal drug substance and after absorption is transported via the blood to the body systemically.

The active agents may be selected from skin whitening or depigmenting agents, anti-acne agents, anti-wrinkle and/or anti-aging agents, pain management agents, agents stimulating healing, emollients, AQP-3 modulating agents, aquaporin modulating agents, proteins from the aquaporin family, collagen synthesis stimulating agents, agents modulating PGC-1-α synthesis, agents modulating the activity of PPARγ, agents which increase or reduce the triglyceride content of adipocytes, agents stimulating or delaying adipocyte differentiation, lipolytic agents or agents stimulating lipolysis, anti-cellulite agents, adipogenic agents, inhibitors of acetylcholine-receptor aggregation, agents inhibiting muscle contraction, anticholinergic agents, elastase inhibiting agents, matrix metalloproteinase inhibiting agents, melanin synthesis stimulating or inhibiting agents, propigmenting agents, self-tanning agents, NO-synthase inhibiting agents, 5α-reductase inhibiting agents, lysyl- and/or prolyl hydroxylase inhibiting agents, antioxidants, free radical scavengers and/or agents against atmospheric pollution, reactive carbonyl species scavengers, anti-glycation agents, antihistamine agents, antiviral agents, antiparasitic agents, skin conditioners, humectants, substances which retain moisture, alpha hydroxy acids, beta hydroxy acids, moisturizers, hydrolytic epidermal enzymes, vitamins, amino acids, proteins, biopolymers, gelling polymers, agents able to reduce or treat the bags under the eyes, exfoliating agents, desquamating agents, keratolytic agents, antimicrobial agents, antifungal agents, fungistatic agents, bactericidal agents, bacteriostatic agents, agents stimulating the synthesis of dermal or epidermal macromolecules and/or capable of inhibiting or preventing their degradation, elastin synthesis-stimulation agents, decorin synthesis-stimulation agents, laminin synthesis-stimulation agents, defensin synthesis-stimulating agents, chaperone synthesis-stimulating agents, cAMP synthesis-stimulating agents, heat shock proteins, HSP70 synthesis stimulators, heat shock protein synthesis-stimulating agents, hyaluronic acid synthesis-stimulating agents, fibronectin synthesis-stimulating agents, sirtuin synthesis-stimulating agents, agents stimulating the synthesis of lipids and components of the stratum corneum (ceramides, fatty acids), agents that inhibit collagen degradation, agents that inhibit elastin degradation, agents that inhibit serine proteases, agents stimulating fibroblast proliferation, agents stimulating keratinocyte proliferation, agents stimulating adipocyte proliferation, agents stimulating melanocyte proliferation, agents stimulating keratinocyte differentiation, agents that inhibit acetylcholinesterase, skin relaxant agents, glycosaminoglycan synthesis-stimulating agents, antihyperkeratosis agents, comedolytic agents, anti-psoriasis agents, anti-dermatitis agents, anti-eczema agents, DNA repair agents, DNA protecting agents, stabilizers, anti-itching agents, agents for the treatment and/or care of sensitive skin, firming agents, redensifying agents, restructuring agents, anti-stretch mark agents, binding agents, agents regulating sebum production, antiperspirant agents, coadjuvant healing agents, agents stimulating reepithelialization, coadjuvant reepithelialization agents, cytokine growth factors, cytokine growth factors, agents which act on capillary circulation and/or microcirculation, calming agents, anti-inflammatory agents, anesthetic agents, agents acting on capillary circulation and/or microcirculation, agents stimulating angiogenesis, agents that inhibit vascular permeability, venotonic agents, agents acting on cell metabolism, agents to improve dermal-epidermal junction, agents inducing hair growth, hair growth inhibiting or retardant agents, chelating agents, plant extracts, essential oils, marine extracts, agents obtained from a biofermentation process, mineral salts, cell extracts, sunscreens and organic or mineral photoprotective agents active against ultraviolet A and/or B rays, and mixtures thereof.

### Skin Whitening and Depigmenting Agents

Exemplary skin-whitening or depigmenting agents include hydrogen peroxide, pyridine-3-carboxamide (nicotinamide), kojic acid, hydroquinine, mulberry root extract, liquorice root extract, Scutellaria baicalensis extract, grape extract, ferulic acid, hinokitiol, arbutin, α-arbutin (bearberry extract), and mixtures thereof. extracts of Achillea millefolium, Aloe vera, Azadirachta indica, Osmunda japonica, Artocarpus incisus, Bidens pilosa, Broussonetia papyrifera, Chlorella vulgaris, Cimicifuga racemosa, Emblica officinalis, Glycyrrhiza glabra, Glycyrrhiza uralensis, Ilex purpurea, Ligusticum lucidum, Ligusticum wallichii, Mitracarpus scaber, Morinda citrifolia, Morus alba, Morus bombycis, Naringi crenulata, Prunus domestica, Pseudostellaria heterophylla, Rumex crispus, Rumex occidentalis, Sapindus mukorossi, Saxifraga sarmentosa, Scutellaria galericulata, Sedum sarmentosum Bunge, Stellaria medica, Triticum Vulgare, Arctostaphylos uva ursi or Withania somnifera, flavonoids, soy extract, lemon extract, orange extract, ginkgo extract, cucumber extract, geranium extract, bearberry (gayuba) extract, carob extract, cinnamon extract, marjoram extract, rosemary extract, clove extract, soluble liquorice extract, blackberry leaf extract, Lipochroman-6™ [INCI: dimethylmethoxy chromanol] and Chromabright™ [INCI: dimethylmethoxy chromanyl palmitate] marketed by Lipotec, Actiwhite™ LS 9808 [INCI: water, glycerin, sucrose dilaurate, polysorbate 20, Pisum sativum (pea) extract] and Dermawhite® NF LS 9410 [INCI: mannitol, arginine HCl, phenylalanine, disodium EDTA, sodium citrate, kojic acid, citric acid, yeast extract] marketed by Laboratoires Serobiologiques/Cognis, Lumiskin™ [INCI: caprylic/capric triglyceride, diacetyl-boldine], Melaclear™ [INCI: glycerin, water, dithiaoctanediol, gluconic acid, sutilains, beta-carotene], O.D.A.white™ [INCI: octadecenedioic acid] and Etioline™ [INCI: glycerin, butylene glycol, Arctostaphylos uva ursi leaf extract, Mitracarpus scaber extract] marketed by Sederma, Sepiwhite™ MSH [INCI: undecylenoyl phenylalanine]marketed by Seppic, Achromaxyl™ [INCI: water, Brassica napus extract] marketed by Vincience, Gigawhite™ [INCI: water, glycerin, Malva sylvestris (mallow) extract, Mentha piperita leaf extract, Primula veris extract, Alchemilla vulgaris extract, Veronica officinalis extract, Melissa officinalis leaf extract, Achillea millefolium extract], Melawhite® [INCI: leukocyte extract, AHA] or Melfade®-J [INCI: water, Arctostaphylos uva-ursi leaf extract, glycerin, magnesium ascorbyl phosphate] marketed by Pentapharm, Albatin® [INCI: 1-aminoethylphosphinic acid, butylene glycol, water] marketed by Exsymol, Tyrostat™-11 [INCI: water, glycerin, Rumex occidentalis extract] and Melanostatine®-5 [INCI: dextran, nonapeptide-1] marketed by Atrium Innovations, arbutin and its isomers, kojic acid and derivatives thereof, ascorbic acid and derivatives thereof such as 6-O-palmitoylascorbic acid, ascorbyl glucoside, dipalmitoyl ascorbic acid, sodium ascorbyl phosphate (NAP), magnesium ascorbyl phosphate (MAP), aminopropyl ascorbyl phosphate, ascorbyl glucoside or ascorbyl tetraisopalmitate (VCIP); retinol and derivatives thereof including tretinoin and isotretinoin, idebenone, hydroxybenzoic acid and derivatives thereof, niacinamide, liquiritin, resorcinol and derivatives thereof, hydroquinone, α-tocopherol, γ-tocopherol, azelaic acid, potassium azeloyl diglycinate, resveratrol, linoleic acid, α-lipoic acid, dihydrolipoic acid, α-hydroxy acids, β-hydroxy acids, ellagic acid, ferulic acid, cinnamic acid, oleanolic acid, aloesin and its derivatives, serine protease inhibitors, for example tryptase, trypsin and PAR-2 inhibitors, and mixtures thereof.

### Anti-Acne Agents

Exemplary anti-acne agents include salicylic acid, glycolic acid, lactobionic acid, azelaic acid, benzoyl peroxide, antibiotics such as Clindamycin, sodium sulfacetamide and erythromycin, retinoids such as adapalene, tazarotene, and tretinoin, which may be sold under trade names such as Retin-A, Differin™, Renova™, and Tazorac™, and mixtures thereof.

Anti-Wrinkle and/or Anti-Aging Agents

Exemplary anti-wrinkle agents and/or anti-aging agents include extracts or hydrolyzed extracts of Vitis vinifera, Rosa canina, Curcuma longa, Iris pallida, Theobroma cacao, Ginkgo biloba, Leontopodium alpinum, Dunaliella salina, synthetic compounds or products, such as Matrixyl® [INCI: palmitoyl pentapeptide-4], Matrixyl® 3000 [INCI: palmitoyl tetrapeptide-7, palmitoyl oligopeptide], Essenskin™ [INCI: calcium hydroxymethionine], Renovage™ [INCI: teprenone] or Dermaxyl™ [INCI: palmitoyl oligopeptide] marketed by Sederma/Croda, Vialox™ [INCI: pentapeptide 3], Syn®-Ake [INCI: dipeptide diaminobutyroyl benzylamide diacetate], Syn®-Coll [INCI: palmitoyl tripeptide-5], Phytaluronate™ [INCI: locust bean (Ceratonia siliqua) gum] or Preregen™ [INCI: Glycine soja (soybean) protein, oxidoreductases] marketed by Pentapharm/DSM, Myoxinol™ [INCI: hydrolyzed Hibiscus esculentus extract], Syniorage™ [INCI: acetyl tetrapeptide-11], Dermican™ [INCI: acetyl tetrapeptide-9] or DN AGE™ LS [INCI: Cassia alata leaf extract] marketed by Laboratoires Serobiologiques/Cognis, Algisium C™ [INCI: methylsilanol mannuronate] or Hydroxyprolisilane CN™ [INCI: methylsilanol hydroxyproline aspartate] marketed by Exsymol, Argireline™ [INCI: acetyl hexapeptide-8], SNAP-7 [INCI: acetyl heptapeptide-4], SNAP-8 [INCI: acetyl octapeptide-3], Leuphasyl® [INCI: pentapeptide-18], Inyline™ [INCI: acetyl hexapeptide-30], Aldenine® [INCI: hydrolyzed wheat protein, hydrolyzed soy protein, tripeptide 1], Preventhelia® [INCI: diaminopropionoyl tripeptide-33], Decorinyl™ [INCI: tripeptide-10 citrulline], Trylagen® [INCI: Pseudoalteromonas ferment extract, hydrolyzed wheat protein, hydrolyzed soy protein, tripeptide 10 citrulline, Tripeptide 1], Eyeseryl® [INCI: acetyl tetrapeptide-5], Peptide AC29 [INCI; acetyl tripeptide-30 citrulline], Relistase™ [INCI: acetylarginyltriptophyl diphenylglycine], Thermostressine™ [INCI: acetyl tetrapeptide-22], Lipochroman 6 [INCI: dimethylmethoxy chromanol], Chromabright™ [INCI: dimethylmethoxy chromanyl palmitate], Antarcticine® [INCI: Pseudoalteromonas ferment extract], dGlyage™ [INCI: lysine HCl, lecithin, tripeptide-9 citrulline], Vilastene™ [INCI: lysine HCl, lecithin, tripeptide-10 citrulline] or Hyadisine™ [INCI: Pseudoalteromonas ferment extract] marketed by Lipotec, Kollaren™ [INCI: tripeptide 1, dextran] marketed by Institut Européen de Biologie Cellulaire, Collaxyl™ IS [INCI: hexapeptide-9], Laminixyl IS™ [INCI: heptapeptide], Orsirtine™ GL [INCI: Oryza sativa (rice) extract], D'Orientine™ IS [INCI: Phoenix dactylifera (Date) seed extract], Phytoquintescine™ [INCI: Einkorn (Triticum monococcum) extract] or Quintescine™ IS [INCI: dipeptide-4] marketed by Vincience/ISP, BONT-L-Peptide [INCI: palmitoyl hexapeptide-19] marketed by Infinitec Activos, Deepaline™ PVB [INCI: palmitoyl hydrolyzed wheat protein] or Sepilift™ DPHP [INCI: dipalmitoyl hydroxyproline] marketed by Seppic, Gatuline™ Expression [INCI: Acmella oleracea extract], Gatuline™ In-Tense™ [INCI: Spilanthes acmella flower extract] and Gatuline™ Age Defense 2 [INCI: Juglans regia (walnut) seed extract] marketed by Gattefossé, Thalassine™ [INCI: algae extract] marketed by Biotechmarine, ChroNOline™ [INCI: caprooyl tetrapeptide-3] and Thymulen-4™ [INCI: acetyl tetrapeptide-2] marketed by Atrium Innovations/Unipex Group, EquiStat™ [INCI: Pyrus malus fruit extract, glycine soja seed extract] or Juvenesce [INCI: ethoxydiglycol and caprylic triglyceride, Retinol, ursolic acid, phytonadione, ilomastat] marketed by Coletica/Engelhard/BASF, Ameliox™ [INCI: carnosine, tocopherol, Silybum marianum fruit extract] and PhytoCellTec™ Malus domestica [INCI: Malus domestica fruit cell culture] marketed by Mibelle Biochemistry, Bioxilift™ [INCI: Pimpinella anisum extract] and SMS Anti-Wrinkle™ [INCI: Annona squamosa seed extract] marketed by Silab, antagonists of the Ca²⁺ channel, such as alverine, manganese or magnesium salts, certain secondary or tertiary amines, retinol and its derivatives, idebenone and its derivatives, Coenzyme Q10 and its derivatives, boswellic acid and its derivatives, GHK and its derivatives and/or salts, carnosine and its derivatives, chloride channel agonists, and mixtures thereof.

For example, U.S. Pat. No. 8,110,207 describes compound of general formula (I)

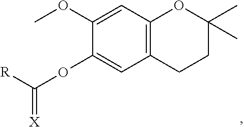

(6-substituted 7-methoxy-2,2-dimethylchromanes), and cosmetically or pharmaceutically acceptable salts, wherein R is a linear or branched, saturated or unsaturated aliphatic group containing 2 to 23 carbon atoms, or a cyclic group, and which can contain substituents selected from hydroxy, alkoxy, amino, carboxyl, cyano, nitro, alkylsulfonyl or halogen atoms; and X is selected from O and S.
Moisturizing Agents, Humectants, Substances that Retain Moisture, and Emollients

Exemplary moisturizing agents, humectants and emollients include sodium pyrrolidone carboxylate; betaines, such as N,N,N-trimethylglycine; yeast extract; polyols and polyethers such as glycerin, ethylhexylglycerin, caprylyl glycol, pentylene glycol, butylene glycol, propylene glycol and their derivatives, triethylene glycol, polyethylene glycol, Glycereth-26, Sorbeth-30; panthenol; pyroglutamic acid and its salts and derivatives; amino acids, such as serine, proline, alanine, glutamate or arginine; ectoine and its derivatives; N-(2-hydroxyethyl)acetamide; N-lauroyl-pyrrolidone carboxylic acid; N-lauroyl-L-lysine; N-alpha-benzoyl-L-arginine; urea; creatine; α- and β-hydroxy acids such as lactic acid, glycolic acid, malic acid, citric acid or salicylic acid, and their salts, such as sodium lactate and lactic acid bacteria fermented solution; polyglyceryl acrylate; sugars and polysaccharides, such as glucose, saccharide isomerate, sorbitol, pentaerythritol, inositol, xylitol, trehalose and derivatives thereof, sodium glucuronate, carraghenates (Chondrus crispus) and chitosan; glycosaminoglycans such as hyaluronic acid and derivatives thereof such as sodium hyaluronate; aloe vera in any of its forms; honey; soluble collagen; lecithin and phosphatidylcholine; ceramides; cholesterol and its esters; tocopherol and its esters, such as tocopheryl acetate or tocopheryl linoleate; long-chain alcohols such as cetearyl alcohol, stearyl alcohol, cetyl alcohol, oleyl alcohol, isocetyl alcohol or octadecan-2-ol; long-chain alcohol esters such as lauryl lactate, myristyl lactate or C₁₂-C₁₅ alkyl benzoates; fatty acids such as stearic acid, isostearic acid or palmitic acid; polyunsaturated fatty acids (PUFAs); sorbitans such as sorbitan distearate; glycerides such as glyceryl monoricinoleate, glyceryl monostearate, glyceryl stearate citrate or caprylic and capric acid triglyceride; saccharose esters such as saccharose palmitate or saccharose oleate; butylene glycol esters, such as dicaprylate and dicaprate; fatty acid esters such as isopropyl isostearate, isobutyl palmitate, isocetyl stearate, isopropyl laurate, hexyl laurate, decyl oleate, cetyl palmitate, di-n-butyl sebacate, isopropyl myristate, isopropyl palmitate, isopropyl stearate, butyl stearate, butyl myristate, isopropyl linoleate, 2-ethylhexyl palmitate, 2-ethylhexyl cocoate, decyl oleate, myristyl myristate; squalene; mink oil; lanolin and its derivatives; acetylated lanolin alcohols; silicone derivatives such as cyclomethicone, dimethicone or dimethylpolysiloxane; Antarcticine® [INCI: Pseudoalteromonas Ferment extract] or acetyl-glutamyl-methionyl-alanyl-isoleucine, acetyl-arginyl-phenylglycyl-phenylglycine or acetyl-arginyl-6-aminohexanoyl-alanine marketed by Lipotec, petrolatum; mineral oil; mineral and synthetic waxes; beeswax (Cera alba); paraffin; or waxes and oils with vegetable origins such as candelilla wax (Euphorbia cerifera), carnauba wax (Copernicia cerifera), shea butter (Butirospermum parkii), cocoa butter (Theobroma cacao), castor oil (Ricinus communis), sunflower oil (Helianthus annuus), olive oil (Olea europaea), coconut oil (Cocos nucifera), palm oil (Elaeis guineensis), wheat germ oil (Triticum vulgare), sweet almond oil (Prunus amygdalus dulcis), musk rose oil (Rosa moschata), soya bean oil (Glycine soja), grape seed oil (Vitis vinifera), calendula oil (Calendula officinalis), jojoba oil (Simmondsia chinensis), mango oil (Mangifera indica), avocado oil (Persea gratissima), and mixtures thereof.

### Anti-Inflammatory Agents

Exemplary anti-inflammatory agents include seal whip extract, Polygonum cuspidatum root extract, allantoin, madecassoside extract, echinacea extract, amaranth seed oil, sandal wood oil, peach tree leaf extract, extract of Aloe vera, Arnica montana, Artemisia vulgaris, Asarum maximum, Calendula officinalis, Capsicum, Centipeda cunninghamii, Chamomilla recutita, Crinum asiaticum, Hamamelis virginiana, Harpagophytum procumbens, Hypericum perforatum, Lilium candidum, Malva sylvestris, Melaleuca alternifolia, Origanum majorana, Origanum vulgare, Prunus laurocerasus, Rosmarinus officinalis, Salix alba, Silybum marianum, Tanacetum parthenium, Thymus vulgaris, Uncaria guianensis or Vaccinium myrtillus, spike moss extract, lysozyme chloride, mometasone furoate, prednisolone, nonsteroidal anti-inflammatories including loxoprofen sodium, flurbiprofen, diclofenac sodium, tiaramide hydrochloride, cyclooxygenase or lipoxygenase inhibitors, benzydamine, acetylsalicylic acid, rosmarinic acid, ursolic acid, glycyrrhizic acid and sodium, potassium and ammonium salts thereof, α-bisabolol, azulene and analogues, sericoside, ruscogenin, escin, scoline, rutin and analogues, hydrocortisone, clobetasol, dexamethasone, prednisone, paracetamol, amoxiprin, benorilate, choline salicylate, faislamine, methyl salicylate, magnesium salicylate, salsalate, diclofenac, aceclofenac, acemetacin, bromfenac, etodolac, indomethacin, oxametacin, proglumetacin, sulindac, tolmetin, ibuprofen, dexibuprofen, carprofen, fenbufen, fenoprofen, flurbiprofen, ketoprofen, dexketoprofen, ketorolac, loxoprofen, naproxen, miroprofen, oxaprozin, pranoprofen, tiaprofenic acid, suprofen, mefenamic acid, meclofenamate, meclofenamic acid, flufenamic acid, tolfenamic acid, nabumetone, phenylbutazone, azapropazone, clofezone, kebuzone, metamizole, mofebutazone, oxyphenbutazone, phenazone, sulfinpyrazone, piroxicam, lornoxicam, meloxicam, tenoxicam, celecoxib, etoricoxib, lumiracoxib, parecoxib, rofecoxib, valdecoxib, nimesulide, naproxcinod, fluproquazone or licofelone, omega-3 and omega-6 fatty acids, morphine, codeine, oxycodone, hydrocodone, diamorphine, pethidine, tramadol, buprenorphine, benzocaine, lidocaine, chloroprocaine, tetracaine, procaine, amitriptyline, carbamazepine, gabapentin, pregabalin, bisabolol, Neutrazen™ [INCI: water, butylene glycol, dextran, palmitoyl tripeptide-8] marketed by Atrium Innovations/Unipex Group, Meliprene® [INCI: dextran, Acetyl Heptapeptide-1] marketed by Institut Européen de Biologie Cellulaire/Unipex Group, Skinasensyl™ [INCI: acetyl tetrapeptide-15] or Anasensyl™ [INCI: mannitol, ammonium glycyrrhizate, caffeine, Hippocastanum (Horse Chestnut) extract] marketed by Laboratoires Serobiologiques/Cognis, Calmosensine™ [INCI: acetyl dipeptide-1] marketed by Sederma, coenzyme Q10 or alkylglyceryl ethers, and mixtures thereof.

### DNA Repair Agents

Exemplary DNA repair agents include C₁-C₈ alkyl tetrahydroxycyclohexanoate, micrococcus lysate, bifida ferment lysate, DNA repair enzymes such as photolyase and T4 endonuclease V, and mixtures thereof.

### Skin Lipid Barrier Repair Agents

Exemplary skin lipid barrier repair agents include phytosphingosine, linoleic acid, cholesterol, and mixtures thereof.

### Anti-Cellulite Agents

Exemplary anti-cellulite agents include Coleus forskohlii root extract, Magnolia grandiflora bark extract, Nelumbo nucifera leaf extract, and mixtures thereof.

### Wound Healing Agents

Exemplary wound-healing agents, coadjuvant healing agents, agents stimulating re-epithelialization and/or coadjuvant re-epithelialization agents include extracts of Aristolochia clematis, Centella asiatica, Rosa moschata, Echinacea angustifolia, Symphytum officinale, Equisetum arvense, Hypericum perforatum, Mimosa tenuiflora, Persea gratissima, Prunus africana, Tormentilla erecta, Aloe vera, soybean protein, Polyplant® Epithelizing [INCI: Calendula officinalis, Hypericum perforatum, Chamomilla recutita, Rosmarinus officinalis] marketed by Provital, Cytokinol® LS 9028 [INCI: hydrolyzed casein, hydrolyzed yeast protein, lysine HCl] marketed by Laboratories Serobiologiques/Cognis or Deliner® [INCI: Zea May (Corn) Kernel extract] marketed by Coletica/Engelhard/BASF, allantoin, cadherins, integrins, selectins, hyaluronic acid receptors, immunoglobulins, fibroblast growth factor, connective tissue growth factor, platelet-derived growth factor, vascular endothelial growth factor, epidermal growth factor, insulin-like growth factor, keratinocyte growth factors, colony-stimulating factors, transforming growth factor beta, tumor necrosis factor alpha, interferons, interleukins, matrix metalloproteinases, cytokines, extra cellular matrices such as collagen I, II, and III, receptor protein tyrosine phosphatases, Antarcticine® [INCI: Pseudoalteromonas ferment extract], Decorinyl® [INCI: Tripeptide-10 citrulline], Trylagen® [INCI: Pseudoalteromonas ferment extract, hydrolyzed wheat protein, hydrolyzed soy protein, tripeptide-10 citrulline, Tripeptide-1], Bodyfensine™ [INCI: acetyl dipeptide-3 aminohexanoate], marketed by Lipotec, and mixtures thereof.

### Muscle Relaxants, Agents Inhibiting Muscle Contraction, Agents Inhibiting Acetylcholine Receptor Clustering and Anticholinergic Agents

Exemplary muscle relaxants, agents inhibiting muscle contraction, agents inhibiting acetylcholine receptor clustering and anticholinergic agents include extracts of Atropa belladonna, Hyoscyamus niger, Mandragora officinarum, Chondrodendron tomentosum, plants of the Brugmansia genus, or the Datura genus, Clostridium botulinum toxin, peptides derived from the protein SNAP-25 or Inyline™ [INCI: acetyl hexapeptide-30] marketed by Lipotec, baclofen, carbidopa, levodopa, bromocriptine, chlorphenesin, chlorzoxazone, donepezil, mephenoxalone, reserpine, tetrabenazine, dantrolene, thiocolchicoside, tizanidine, clonidine, procyclidine, glycopyrrolate, atropine, hyoscyamine, benztropine, scopolamine, promethazine, diphenhydramine, dimenhydrinate, dicyclomine, cyclobenzaprine, orphenadrine, flavoxate, cyclopentolate, ipratropium, oxybutynin, pirenzepine, tiotropium, trihexyphenidyl, tolterodine, tropicamide, solifenacin, darifenacin, mebeverine, trimethaphan, atracurium (besylate), cisatracurium, doxacurium, fazadinium, metocurine, mivacurium, pancuronium, pipecuronium, rapacuronium, tubocurarine, dimethyl tubocurarine, rocuronium, vecuronium, suxamethonium, 18-methoxycoronaridine, carisoprodol, febarbamate, meprobamate, metocarbamol, phenprobamate, tibamate, anticonvulsant agents such as levetiracetam, stiripentol, phenobarbital, methylphenob arbital, pentobarbital, metharbital, barbexaclone, primidone, carbamazepine, oxcarbazepine, benzodiazepines, for example clonazepam, cloxazolam, clorazepate, diazepam, flutoprazepam, lorazepam, midazolam, nitrazepam, nimetazepam, phenazepam, temazepam, tetrazepam, clobazam, hydrochloric acid epihydrochloride, talipexole hydrochloride, tolperisone hydrochloride, and mixtures thereof.

### Pain Management Agents

Exemplary pain management agents and local anesthetics include lidocaine and salts such as lidocaine hydrochloride, bupivacaine and bupivacaine hydrochloride, mepivacaine and mepivacaine hydrochloride, etidocaine, prilocaine and prilocaine hydrochloride, tetracaine, procaine, chloroprocaine, benzocaine, and their salts; counterirritant agents that mask pain such as menthol, camphor, methylsalicylate, cinnamaldehyde, capsaicin and mixtures thereof, acetylsalicylic acid (aspirin) and other salicylic acid esters, diclofenac and salts thereof such as sodium, diethylamine, ibuprofen, ketoprofen, acetaminophen and other non-steroidal anti-inflammatory drugs, analgesic drugs such as morphine hydrochloride, fentanyl citrate, buprenorphine hydrochloride, and the like, and mixtures thereof.

Hair Growth Retardation and Stimulation Agents

Exemplary hair growth retardation agents include ursolic acid, Boswellia serrata extract, activin and activin agonists, flavonoids such as quercetin, curcumin, galangin, fisetin, myricetin, apigenin; propyl gallate, nordihydroguaiaretic acid, caffeic acid, tyrosine kinase inhibitors such as lavendustin, erbstatin, tyrphostins, benzoquinone-ansamycin herbimycin A, thiazolidinediones, phenazocine, 2,3-dihydro-2-thioxo-1H-indol-3-alkanoic acids, phenothiazine derivatives such as thioridazine; sphingosine and derivatives thereof such as phytosphingosine; staurosporine and derivatives thereof, glycyrrhetinic acid, lauryl isoquinolinium bromide, Decelerine™ [INCI: lauryl isoquinolinium bromide, Pseudoalteromonas ferment extract] marketed by Lipotec, serine protease inhibitors, trypsin, and mixtures thereof.

Exemplary hair growth stimulating agents include Serenoa serrulata fruit extract, licorice extract, Tussilago farfara or Achillea millefolium, nicotinic acid esters such as C₃-C₆ alkyl nicotinates such as methyl or hexyl nicotinate, benzyl nicotinate, or tocopheryl nicotinate; biotin, 5α-reductase-inhibiting agents, anti-inflammatory agents, retinoids, for example all-trans-retinoic acid or tretinoin, isotretinoin, retinol or vitamin A, and derivatives thereof, such as zinc salt of acetate, palmitate, propionate, motretinide, etretinate and trans-retinoate; anti-bacterial agents, calcium channel blockers, for example cinnarizine and diltiazem; hormones, for example estriol and its analogues and thyroxine and its analogues and/or salts; antiandrogenic agents, for example oxendolone, spironolactone and diethylstilbestrol; anti-radical agents, esterified oligosaccharides, for example those described in documents EP 0211610 and corresponding U.S. Pat. No. 4,761,401 and EP 0064012 and corresponding U.S. Pat. No. 4,607,025; derivatives of hexosaccharic acids, for example glucosaccharic acid or those described in EP 0375388 and corresponding U.S. Pat. No. 5,081,151; glucosidase inhibitors, for example D-glucaro-1,5-lactam and those described in document EP 0334586 and corresponding U.S. Pat. No. 4,975,441; glycosaminoglycanase and proteoglycanase inhibitors, for example L-galactono-1,4-lactone and those described in document EP 0277428 and corresponding U.S. Pat. No. 5,015,470; tyrosine kinase inhibitors, for example 1-amido-1-cyano(3,4-dihydroxyphenyl)ethylene and those described in document EP 0403238 and corresponding U.S. Pat. No. 5,124,354, diazoxides, for example 7-(acetylthio)-4′,5′-dihydrospiro[androst-4-ene-17,2′-(3H)furan]-3-one, 1,1-dioxide of 3-methyl-7-chloro[2H]-1,2,4-benzothiadiazine and spirooxazine; phospholipids, for example lecithin; salicylic acid and derivatives thereof, hydroxycarboxylic and keto carboxylic acids and esters thereof, lactones and their salts; anthralin, eicosa-5,8,11-trienoic acids and esters thereof and amides among others, minoxidil and derivatives, acetyl glucosamine, and mixtures thereof.

Agents for Reducing Bags Under the Eyes

Exemplary agents for reducing bags under the eye and dark circles include hesperidin methyl chalcone, dipeptide-2, Passiflora incarnate flower extract, linoleic acid, isolinoleic acid, peptides as described in U.S. 20100098769, and mixtures thereof.

Collagen Synthesis or Blood Circulation Enhancing Agents

Exemplary collagen synthesis or blood circulation enhancing agents include arginine, Ascophyllum nodosum extract, Asparagopsis armata extract, and mixtures thereof.

Antioxidants

Exemplary antioxidants include nordihydroguaiaretic acid, butylhydroxyanisole (BHA), butylhydroxytoluene (BHT), propyl gallate, erythorbic acid, sodium erythorbate, para-hydroxyanisole, tert-butylhydroquinone (TBHQ), 2,6-di-tert-butyl-4-methylphenol, gallic acid esters such as propyl gallate and octyl gallate, probucol, polyphenols, ascorbic acid and its salts, enzymes such as catalase, superoxide dismutase and peroxidases; citric acid, citrates, monoglyceride esters, calcium metabisulfite, lactic acid, malic acid, succinic acid, tartaric acid, vitamin A or β-carotene, vitamins E and C, tocopherols such as vitamin E acetate, ascorbic acid esters such as ascorbyl palmitate and ascorbyl acetate, zinc, copper, mannitol, reduced glutathione, carotenoids such as cryptoxanthin, astaxanthin and lycopene; cysteine, uric acid, carnitine, taurine, tyrosine, lutein, zeaxanthin, N-acetyl-cysteine, carnosine, γ-glutamylcysteine, quercetin, lactoferrin, dihydrolipoic acid, tea catechins, retinyl palmitate and derivatives thereof, bisulfate, metabisulfite and sodium sulfite, chromans, chromenes and their analogues, Lipochroman-6 [INCI: Dimethylmethoxy Chromanol], chelating agents of metals such as EDTA, sorbitol, phosphoric acid or dGlyage™ [INCI: Lysine HCl, Lecithin, Tripeptide-9 Citrulline]; extract of Ginkgo Biloba, plant extracts such as sage, pomegranate, rosemary, oregano, ginger, marjoram, cranberry, grape seed, tomato, green leaf tea and black leaf tea; oleoresin extract, extract of plants which contain phenols such as vanillin, ellagic acid and resveratrol; tertiary butylhydroquinone or mixtures thereof, metal salts with a valence of 2 such as selenium, cadmium, vanadium or zinc; α-lipoic acid, coenzyme Q, idebenone and derivatives thereof, and mixtures thereof.

Antihistamine Agents

Exemplary antihistamine agents include chlorpheniramine maleate, promethazine hydrochloride, cetirizine hydrochloride, and mixtures thereof.

UV Absorbers

Exemplary ultraviolet ray absorbers and agents capable of filtering UV rays include benzophenone derivatives such as 2,4-dihydroxybenzophenone, organic and mineral photoprotective agents active against A and/or B ultraviolet rays such as substituted benzotriazoles, substituted diphenylacrylates, organic nickel complexes, umbelliferone, urocanic acid, biphenyl derivatives, stilbene, 3-benzylidene camphor, and derivatives thereof such as 3-(4-methylbenzylidene)camphor; 4-aminobenzoic acid and derivatives thereof, 2-ethylhexyl 4-(dimethylamino)benzoate, 2-octyl 4-(dimethylamino)benzoate and amyl 4-(dimethylamino)benzoate; cinnamic acid derivatives such as benzyl cinnamate, cinnamic acid esters, such as 2-ethylhexyl 4-methoxycinnamate and diethylamino hydroxybenzoyl hexyl benzoate, propyl 4-methoxycinnamate, isoamyl 4-methoxycinnamate, 2-ethylhexyl-2-cyano-3,3-diphenyl cinnamate (octocrylene); salicylic acid derivatives such as benzyl salicylate and salicylic acid esters, such as 2-ethylhexyl salicylate, 4-isopropylbenzyl salicylate, homomenthyl salicylate; benzophenone derivatives, such as 2-hydroxy-4-methoxybenzophenone, 2-hydroxy-4-methoxy-4′-methylbenzophenone, and 2,2′-dihydroxy-4-methoxybenzophenone; benzalmalonic acid esters, such as di-2-ethylhexyl 4-methoxybenzalmalonate; triazine derivatives, such as 2,4,6-trianilino-(p-carbo-2′-ethyl-1′-hexyloxy)-1,3,5-triazine, octyl triazone or diethylhexyl butamido triazone; propane-1,3-diones, such as 1-(4-tert-butylphenyl)-3-(4′-methoxyphenyl)propane-1,3-dione; ketotricyclo(5.2.1.0)decane derivatives; 2-phenylbenzimidazole-5-sulfonic acid; benzophenone sulfonic acid derivatives, such as 2-hydroxy-4-methoxybenzophenone-5-sulfonic acid and its salts; 4-(2-oxo-3-bornylidenemethyl)benzenesulfonic acid, benzoyl methane derivatives, such as benzoyl methane 2-methyl-5-(2-oxo-3-bornylidene)sulfonic acid, such as 1-(4′-tert-butylphenyl)-3-(4′-methoxyphenyl)propane-1,3-dione, 4-tert-butyl-4′-methoxydibenzoylmethane, 1-phenyl-3-(4′-isopropylphenyl)-propane-1,3-dione, enamine compounds, anthranilates, silicons, benzimidazole derivatives, imidazolines, benzoyl derivatives, Chromabright™ [INCI: dimethylmethoxy chromanyl palmitate] and Preventhelia® [INCI: diaminopropionoyl tripeptide-33] both marketed by Lipotec, metal oxides such as zinc oxide, titanium, iron, zirconium, silicon, manganese, aluminum and cerium; silicates, talc, barium sulfate, zinc stearate, carbon nanotubes, and mixtures thereof.

Amino Acids and their Salts

Exemplary amino acids include glycine, alanine, valine, leucine, isoleucine, serine, threonine, phenylalanine, tyrosine, tryptophan, cystine, cysteine, methionine, citrulline, proline, hydroxyproline, aspartic acid, asparagine, glutamic acid, glutamine, arginine, histidine, lysine, γ-aminobutyric acid, salts thereof and mixtures thereof. Example salts include glutamate, trisodium methylglycine diacetate (e.g., Trilon® M marketed by BASF), derivatives of amino acids which contain cysteine, in particular N-acetyl cysteine, ergothioneine or S-carboxymethylcysteine, and/or mixtures thereof.

Peptides and Commercial Formulations Containing them

Exemplary peptides and commercial mixtures which contain them some of which are mentioned elsewhere herein for particular effects, and may include wheat peptides, soybean peptide, copper peptide GHK-Cu [INCI: Tripeptide-1], acetyl-glutamyl-methionyl-alanyl-isoleucine, acetyl-arginyl-phenylglycyl-phenylglycine, Bodyfensine™ [INCI: acetyl dipeptide-3 aminohexanoate], Relistase™ [INCI: acetyl arginyltriptophyl diphenylglycine], acetyl-arginyl-phenylglycyl-valyl-glycine, acetyl-arginyl-phenylglycyl-valyl-phenylglycine, diaminopropionyl-alanyl-asparaginyl-histidine, acetyl-arginyl-asparaginyl-histidyl-citrulline-amide, Aldenine® [INCI: hydrolyzed wheat protein, hydrolyzed soy protein, tripeptide-1], Decorinyl® [INCI: tripeptide-10 citrulline], Serilesine®[INCI: hexapeptide-10], Peptide AC29 [INCI: acetyl tripeptide-30 citrulline], Vilastene™ [INCI: lysine HCl, lecithin, tripeptide-10 citrulline], dGlyage™ [INCI: Lysine HCl, Lecithin, Tripeptide-9 Citrulline], Eyeseryl® [INCI: acetyl tetrapeptide-5], Preventhelia® [INCI: diaminopropionoyl tripeptide-33], Argireline® [INCI: acetyl hexapeptide-8], SNAP-7 [INCI: acetyl heptapeptide-4], SNAP-8 [INCI: acetyl octapeptide-3], Leuphasyl® [INCI: pentapeptide-18], Trylagen® [INCI: Pseudoalteromonas ferment extract, hydrolyzed wheat protein, hydrolyzed soy protein, tripeptide-10 citrulline, tripeptide-1], Inyline™ [INCI: acetyl hexapeptide-30], Melatime™ [INCI: acetyl tripeptide-40], Thermostressine™ [INCI: acetyl tetrapeptide-22] and Liporeductyl® [INCI: caffeine, Butcher's broom (Ruscus Aculeatus) root extract, triethanolamine-hydroiodide, carnitine, Ivy (Hedera helix) extract, escin, tripeptide-1] marketed by Lipotec, Matrixyl® [INCI: Palmitoyl Pentapeptide-4], Matrixyl® 3000 [INCI: palmitoyl tetrapeptide-7, palmitoyl oligopeptide], Dermaxyl® [INCI: palmitoyl oligopeptide], Calmosensine™ [INCI: acetyl dipeptide-1], Biopeptide CL™ [INCI: glyceryl polymethacrylate, propylene glycol, palmitoyl oligopeptide] and Biopeptide EL™ [INCI: palmitoyl oligopeptide] marketed by Sederma, pseudodipeptides, IP 2000 [INCI: dextran, trifluoroacetyl tripeptide-2] marketed by IEB and Atrium, Pepha®-TIMP [INCI: Human Oligopeptide-20], ECM-Protect® [INCI: Water (water), dextran, Tripeptide-2] and Melanostatine®-5 [INCI: dextran, nonapeptide-1] marketed by Atrium Innovations, TIMP-Peptide™ [proposed INCI: acetyl hexapeptide], Bronzing S.F. [proposed INCI: butyryl pentapeptide], BONT-L-Peptide [INCI: Palmitoyl Hexapeptide-19] and ECM Moduline [proposed INCI: Palmitoyl tripeptide-28] marketed by Infinitec Activos, IP2000™ [INCI: dextran, Trifluoroacetyl tripeptide-2] marketed by Institut Européen de Biologie Cellulaire, Syn®-Coll [INCI: Palmitoyl Tripeptide-5] marketed by Pentapharm, Neutrazen™ [INCI: Water, butylene Glycol, dextran, Palmitoyl Tripeptide-8], ChroNOline™ [INCI: Caprooyl Tetrapeptide-3] and Thymulen-4 [INCI: Acetyl Tetrapeptide-2] marketed by Atrium Innovations/Unipex Group, Meliprene® [INCI: dextran, Acetyl Heptapeptide-1] and Melitane® [INCI: Acetyl Hexapeptide-1] marketed by Institut Européen de Biologie Cellulaire/Unipex Group, Skinasensyl™ [INCI: Acetyl Tetrapeptide-15] marketed by Laboratoires Serobiologiques/Cognis, Vialox® [INCI: Pentapeptide-3], Syn®-Ake® [INCI: Dipeptide Diaminobutyroyl Benzylamide Diacetate], Syn®-Coll [INCI: Palmitoyl Tripeptide-5], Syniorage™ [INCI: Acetyl Tetrapeptide-11], Dermican™ [INCI: Acetyl Tetrapeptide-9] marketed by Laboratoires Sérobiologiques/Cognis, Kollaren®[INCI: Tripeptide-1, dextran] marketed by Institut Européen de Biologie Cellulaire, Collaxyl® IS [INCI: Hexapeptide-9], Laminixyl IS™ [INCI: Heptapeptide], Quintescine™ IS [INCI: Dipeptide-4], UCPeptide™ V [INCI: Pentapeptide] and AT Peptide™ IS [INCI: Tripeptide-3] marketed by Vincience/ISP, glutathione, carnosine and/or mixtures thereof; and peptides of pharmaceutical use, such as glucagon, leuprolide, goserelin, triptorelin, buserelin, nafarelin, deslorelin, histrelin, avorelin, abarelix, cetrorelix, ganirelix, degarelix, desmopressin, somatostatin and analogues of somatostatin such as octreotide, vapreotide and lanreotide, among others.

Specific examples of peptides include those described in the following U.S. Publications, patents, and international applications, where in each case, R₁ and R₂ are respective N and C peptide terminating groups which are generally not α-amino acids, examples of which are given in the respective patent documents:

U.S. Pat. No. 6,169,074, which describes an isolated excitation-secretory uncoupling peptide (ESUP) for inhibiting neurotransmitter secretion from neuronal cells, consisting of the amino acid sequence of SEQ. ID. NO.: 4 (170-EIDTQNRQIDRIMEKADSNKTRIDEANQRATKMLGSG-206, which is the amino acid sequence of the substrate binding domain of SNAP-25), SEQ. ID. NO.: 7 (170-EIDTQNRQIDRIMEKADSNK-189, which is the amino acid sequence of ESUP/E20h), SEQ. ID. NO.: 8 (181-IMEKADSNKTRIDEANQRATKMLGSG-206, which is the amino acid sequence of ESUP/E26h), SEQ. ID. NO.: 9 (87-SNKTRIDEANQRATKMLGSG-206, the amino acid sequence of ESUP/A20h), and SEQ. ID. NO.: 12 (Gln-Asn-Arg-Gln-Ile-Asp-Arg-Ile-Met-Glu-Lys-Ala-Asp-Ser-Asn-Lys, the amino acid sequence of an ESUP derived from SNAP-25). All residues correspond to substrate binding domain residues.

U.S. Pat. Nos. 7,015,192 and 7,473,679, which describe peptides having a sequence at least 3 and no more than 30 adjacent amino acids from the amino end of protein SNAP-25 and which is useful as neuronal exocytosis inhibitor, in particular, the synthetic peptide whose complete amino acid sequence is selected from the amino acid sequence of SEQ ID NO: 2 (Glu Glu Met Gln Arg Arg) and the amino acid sequence of SEQ ID NO: 3 (Glu Leu Glu Glu Met Gln Arg Arg Ala Asp Gln Leu Ala). The N-terminus of the peptide may be acetylated and the amino acid at the C-terminus of the peptide may be amidated.

U.S. Pat. No. 7,943,156, which describes peptides capable of increasing firmness of skin and delaying aging of skin. These XIKVAV peptides of general formula (III): X-SEQ ID NO. 1-Y:

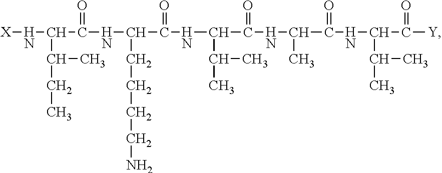

wherein X is selected from the group consisting of hydrogen, an amino acid and an acyl group and Y is selected from the group consisting of amino, hydroxyl and thiol. The XIKVAV peptides of general formula X-SEQ ID NO. 1-Y stimulate bioadhesion of cutaneous cells by increasing expression of bioadhesion peptides.

U.S. 20100021510, which describes a peptide capable of regulating neuronal exocytosis, of the general formula (IV): R₁-AA-R₂ its stereoisomers, mixtures thereof, and its cosmetically and pharmaceutically acceptable salts, wherein AA is a sequence of a leas 3 and up to 40 adjacent amino acids contained in the amino acid sequence SEQ ID No.: 1 selected from MAEDADMRNELEEMQRRADQL, ADESLESTRRMLQLVEESKDAGI, ELEEMQRRADQLA, ELEEMQRRADQL, ELEEMQRRADQ, ELEEMQRRAD, ELEEMQRRA, ELEEMQRR, LEEMQRRADQL, LEEMQRRADQ, LEEMQRRAD, LEEMQRRA, LEEMQRR, EEMQRRADQL, EEMQRRADQ, EEMQRRAD, EEMQRRA, EEMQRR, LESTRRMLQLVEE, NKDMKEAEKNLT, KNLTDL, IMEKADSNKTRIDEANQRATKMLGSG, SNKTRIDEANQRATKMLGSG, TRIDEANQRATKMLGSG, DEANQRATKMLGSG, NQRATKMLGSG and QRATKMLGSG, SEQ ID No.: 1 being Met Ala Glu Asp Ala Asp Met Arg Asn Glu Leu Glu Glu Met Gln Arg Ala Asp Gln Leu Ala Asp Glu Ser Leu Glu Ser Thr Arg Arg Met Leu Gln Leu Val Glu Glu Ser Lys Asp Ala Gly Ile Arg Thr Leu Val Met Leu Asp Glu Gln Gly Glu Gln Leu Glu Arg Ile Glu Glu Gly Met Asp Gln Ile Asn Lys Asp Met Lys Glu Ala Glu Lys Asn Leu Thr Asp Leu Gly Lys Phe Cys Gly Leu Cys Val Cys Pro Cys Asn Lys Leu Lys Ser Ser Asp Ala Tyr Lys Lys Ala Trp Gly Asn Asn Gln Asp Gly Val Val Ala Ser Gln Pro Ala Arg Val Val Asp Glu Arg Glu Gin Met Ala Ile Ser Gly Gly Phe Ile Arg Arg Val Thr Asn Asp Ala Arg Glu Asn Glu Met Asp Glu Asn Leu Glu Gln Val Ser Gly Ile Ile Gly Asn Leu Arg His Met Ala Leu Asp Met Gly Asn Glu Ile Asp Thr Gln Asn Arg Gln Ile Asp Arg Ile Met Glu Lys Ala Asp Ser Asn Lys Thr Arg Ile Asp Glu Ala Asn Gln Arg Ala Thr Lys Met Leu Gly Ser Gly.

U.S. 20100098769, which describes a peptide capable of reducing or removing bags formed under the eyes of general formula (V):

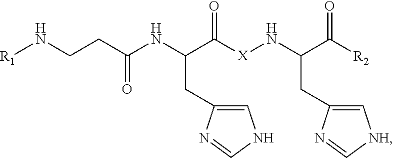

its stereoisomers, mixtures thereof, and its cosmetically and dermopharmaceutically acceptable salts, where X is selected from cysteinyl, seryl, threonyl and aminobutyryl.

U.S. 20110002969, which describes a peptide which includes only four amino acids and which is capable of inhibiting the activity of matrix metalloproteinases, of general formula (VI): R₁-AA₁-AA₂-AA₃-AA₄-R₂, stereoisomers thereof, mixtures thereof and cosmetically or pharmaceutically acceptable salts thereof, wherein: AA₁ is -Arg-; AA₂ is selected from -His- and -Asn-; AA₃ is selected from -His- and -Arg-; AA₄ is -Cit-, Specific examples include R1-Arg-His-His-Cit-R₂, R₁-Arg-Asn-Arg-Cit-R₂, and stereoisomers, mixtures thereof and/or cosmetic or pharmaceutical acceptable salts thereof.

U.S. 20090155317, which describes a peptide which includes only four amino acids and which is capable of reducing facial wrinkles, of general formula (VII):

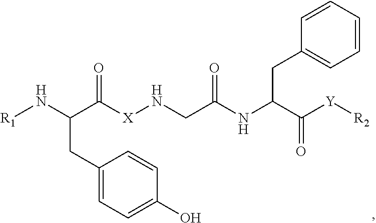

and cosmetically or dermopharmaceutically acceptable salts thereof, wherein: X and Y are selected from natural amino acids in their L- or D-form and non-encoded amino acids. Specific examples include peptides where X is glycyl, D-alanyl or D-seryl, and/or where Y is L-methionyl or L-leucyl.

U.S. 20110195102, which describes a peptide of only four amino acids, which is capable of inhibiting the activity of Reactive Carbonyl Species (RCS) with general formula (VIII): R₁-AA₁-AA₂-AA₃-AA₄-R₂, its stereoisomers, mixtures thereof, and its cosmetically or pharmaceutically acceptable salts, wherein AA₁ is selected from -Lys-, -Orn-, -Dab-, -Dpr-, -Agl-, -3,4-dehydrolysine and -4,5-dehydrolysine; AA₂ is -Ala-; AA₃ is selected from -Asp-, -Ala-, -Asn-, -Glu- and -Pro-; and AA₄ is -His-. Specific examples include R₁-L-Dpr-D-Ala-L-Ala-L-His-R₂, R₁-L-Dpr-D-Ala-L-Pro-L-His-R₂, R₁-L-Dpr-L-Ala-L-Pro-L-His-R₂, and stereoisomers, mixtures thereof and/or cosmetic or pharmaceutical acceptable salts thereof.

U.S. 20110300199, which describes a peptide having a maximum of seven amino acids which is capable of inhibiting elastase activity and/or stimulating collagen synthesis in the skin of general formula (IX): R₁—Wₚ—Xₙ-AA₁-AA₂-AA₃-AA₄-Yₘ—R₂, its stereoisomers, mixtures thereof, and/or its cosmetically or pharmaceutically acceptable salts, wherein at least one of the amino acids AA₁, AA₂ and AA₄ is uncoded; AA₁ is selected from -Arg-, -Phg- and -Nle- or is absent; AA₂ is selected from -Ala-, -Phg-, -Cit- and -Nle-; AA₃ is selected from -Trp-, -Val- and -Tyr-; AA₄ is selected from -Phg- and -Gly-; W, X and Y are independently selected from the group consisting of coded and uncoded amino acids; and p, n and m each range between 0 and 1. Specific examples include R1-L-Arg-L-Nle-L-(or D-)-Phg-L-Tyr-L-(or D)-Phg-R₂, R₁-L-Arg-(or -L-Nle- or absent)-L-(or D-)-Phg-L-Tyr-L-(or D)-Phg-R₂, R₁-L-Arg-L-(or D-)-Phg-L-Val-L-(or D-)-Phg (or -L-Gly-)R₂, and R₁-L-(or D-)-Phg-L-(or D-)-Phg-L-Trp-L-(or D-)-Phg-R₂, and corresponding peptides wherein at least one of W, X, and Y is present, and stereoisomers, mixtures thereof and/or cosmetic or pharmaceutical acceptable salts thereof.

U.S. 20120021029, which describes a peptide having only three amino acids of general formula (X): R₁-AA₁-AA₂-AA₃-R₂, its stereoisomers, mixtures thereof and/or its cosmetic or pharmaceutical acceptable salts, wherein: AA₁ and AA₂ are independently selected from -Tyr- and -Phe-; and AA₃ is selected from -Nle- and -Met-. Specific examples include R₁-L-Tyr-L-Tyr-L-Met-R₂, R₁-L-Tyr-L-Phe-L-Met-R₂, and R₁-L-Tyr-L-Tyr-L-Nle-R₂, and stereoisomers, mixtures thereof and/or cosmetic or pharmaceutical acceptable salts thereof.

U.S. 20120121675, which describes a peptide of general formula (XI): R₁—Wₙ—Xₘ-AA₁-AA₂-AA₃-AA₄-AA₅-AA₆-Yₚ—Zₛ—R₂, its stereoisomers, mixtures thereof and/or its cosmetic or pharmaceutical acceptable salts, wherein AA₁ is selected from Asp, Glu and Pro; AA₂ is Asp; AA₃ is selected from Tyr and Arg; AA₄ is selected from Phe and Tyr; AA₅ is selected from Arg and Lys; AA₆ is selected from Leu and Met; W, X, Y and Z are independently selected from coded amino acids and non-coded amino acids; n, m, p and s independently have a value of between 0 and 1. Specific examples include R₁-L-Glu-L-Asp-L-Tyr-L-Tyr-L-Arg-L-Leu-R₂, R₁-L-Pro-L-Asp-L-Tyr-L-Tyr-L-Lys-L-Leu-R₂, R₁-L-Glu-L-Asp-L-Arg-L-Phe-L-Arg-L-Met-R₂, R₁-L-Glu-L-Asp-L-Tyr-L-Tyr-L-Arg-L-Met-R₂, and R₁-L-Pro-L-Asp-L-Tyr-L-Tyr-L-Arg-L-Met-R₂, and corresponding peptides wherein at least one of W, X, Y and Z is present, and stereoisomers, mixtures thereof and/or cosmetic or pharmaceutical acceptable salts thereof.

U.S. 20130101662, which describes a peptide of general formula (XII): R1-Wₙ—Xₘ-AA₁-AA₂-AA₃-AA₄-Yₚ—Z_{q}—R², its stereoisomers, mixtures thereof and/or its cosmetically or pharmaceutically acceptable salts, wherein: AA₁ is -His-; AA₂ is selected from the group consisting of -His-, -Leu- and -Pro-; AA₃ is -Leu-; AA₄ is selected from the group consisting of -Arg- and -Asn-; W, X, Y and Z are independently selected from amongst themselves from the group consisting of the codified amino acids and uncodified amino acids; n, m, p and q are independently selected from amongst themselves and have a value between 0 and 1; n+m+p+q is less or equal to 2. Specific examples include R₁-L-His-L-Leu-L-Leu-L-Arg-R₂ and R₁-L-His-L-Pro-L-Leu-L-Arg-R₂.

U.S. 20130309281, which describes a peptide of general formula (XIII):

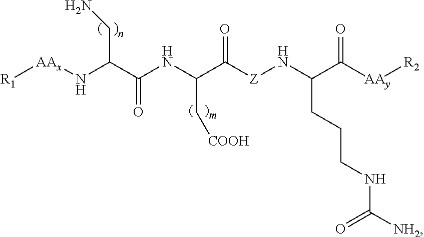

stereoisomers thereof, mixtures thereof, and cosmetically and dermopharmaceutically acceptable salts thereof, wherein: Z is selected from the group consisting of alanyl, allo-isoleucyl, glycyl, isoleucyl, isoseryl, isovalyl, leucyl, norleucyl, norvalyl, prolyl, seryl, threonyl, allo-threonyl or valyl; n and m range independently from one another between 1 and 5; AA is selected from the group consisting of natural encoded amino acids in their L- or D-form and non-encoded amino acids; x and y range independently from one another between 0 and 2. Specific examples include those where wherein Z is L-isoleucyl, L-threonyl or L-valyl and wherein x and y are 0, and stereoisomers, mixtures thereof and/or cosmetic or pharmaceutical acceptable salts thereof.

U.S. 20140120141, which describes a peptide of general formula (XIV): R₁—Wₙ—Xₘ-AA-AA₂-AA₃-AA₄AA₄-AA-AA₆-Yₚ—Z_{q}—R₂ its stereoisomers, mixtures thereof and/or its cosmetic or pharmaceutical acceptable salts, wherein: AA₁ is selected from the group consisting of -Ser-, -Thr- and -Tyr-; AA₂ is selected from the group consisting of -Pro- and -Val-; AA₃ is -Ala-; AA₄ is selected from the group consisting of -Glu-, -Gly- and -Val-; AA₅ is -Gly-; AA₆ is selected from the group consisting of -Gln-, -Gly-, -His- and -Pro-; W, X, Y, Z are amino acids and are independently selected from amongst themselves; n, m, p and q are independently selected from amongst themselves and have a value of 0 or 1; n+m+p+q is lower than or equal to 2. Specific examples include R₁-L-Tyr-L-Pro-L-Ala-L-Glu-L-Gly-L-Gln-R₂, R₁-L-Ser-L-Val-L-Ala-L-Val-L-Gly-L-Gln-R₂, and R₁-L-Ser-L-Pro-L-Ala-L-Gly-L-Gly-L-Pro-R₂, and stereoisomers, mixtures thereof and/or cosmetic or pharmaceutical acceptable salts thereof.

U.S. 20150183823, which describes peptides of general formula (XV): R₁-AA₁-AA₂-AA₃-R₂, where AA₁ is selected from -Tyr- and -Phe-, AA₂ is -Tyr-, and AA₃ is selected from -Nle- and -Met-, its stereoisomers, mixtures thereof and/or their cosmetically or pharmaceutically acceptable salts, which is suited to the treatment and/or care of conditions, disorders and/or diseases of the skin and/or hair by stimulating cyclic adenosine monophosphate synthesis (cAMP). Specific examples include R₁-L-Tyr-L-Tyr-L-Met-R₂, R₁-L-Tyr-L-Phe-L-Met-R₂, and R₁-L-Tyr-L-Tyr-L-Nle-R₂, and stereoisomers, mixtures thereof and/or cosmetic or pharmaceutical acceptable salts thereof.

WO2014/086785 (and U.S. Ser. No. 14/649,747; Filed Jun. 4, 2015), which describes compounds capable of accelerating the DNA protection and repair processes of general formula (XVII):

R1-Wₙ—Xₘ-AA₁-AA₂-AA₃-AA₄-AA₅-AA₆-Yₚ—Z_{q}—R₂, its stereoisomers, mixtures thereof and/or its cosmetically or pharmaceutically acceptable salts, wherein AA₁ is -Tyr-; AA₂ is selected from -Asn-, -His-, -Tyr- and -Glu-; AA₃ is selected from -Lys-, -Ser- and -Pro-; AA₄ is selected from -Gly-, -Leu-, -Lys- and -His-; AA₅ is selected from -Gln- and -Asn-; AA₆ is -Val-; W, X, Y, Z are independently selected from amino acids. n, m, p and q independently have a value of 0 or 1; n+m+p+q is smaller than or equal to 2.

WO2014/170347 (and U.S. application Ser. No. 14/783,689, filed Oct. 9, 2015), which describes a compound of general formula (XVI):

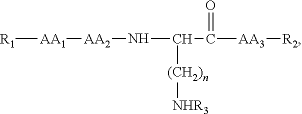

its stereoisomers, mixtures thereof and/or its cosmetic or pharmaceutical acceptable salts, wherein AA₁ is selected from -Asp-, -Glu-, -Asn-, -Gln-, -Lys- and -Gly-, AA₂ is selected from -Val-, -Leu-, -Ile-, -Met-, -Cit-, -His-, -Thr- and -Gln-; AA₃ is selected from -Tyr-, -Trp- and 4-Abz; n is selected from 1, 2, 3 and 4, R₃ is selected from H and -AA₂-AA₁-R₁, R₁ is selected from H, a polymer derived from polyethylene glycol, substituted or unsubstituted non-cyclic aliphatic groups, substituted or unsubstituted alicyclyl groups, substituted or unsubstituted heterocyclyl groups, substituted or unsubstituted heteroarylalkyl groups, substituted or unsubstituted aryl groups, substituted or unsubstituted aralkyl groups and R₆—CO—, wherein R₆ is selected from H, substituted or unsubstituted non-cyclic aliphatic groups, substituted or unsubstituted alicyclyl groups, substituted or unsubstituted aryl groups, substituted or unsubstituted aralkyl groups, substituted or unsubstituted heterocyclyl groups and substituted or unsubstituted heteroarylalkyl groups; R₂ is selected from —NR₄R₅, —OR₄ and —SR₄, wherein R₄ and R₅ are independently selected from H, a polymer derived from polyethylene glycol, substituted or unsubstituted non-cyclic aliphatic group, substituted or unsubstituted alicyclyl, substituted or unsubstituted heterocyclyl, substituted or unsubstituted heteroarylalkyl, substituted or unsubstituted aryl, and substituted or unsubstituted aralkyl; and R₁ and/or R₂ are not α-amino acids.

Vitamins

Example vitamins and factors acting like a vitamin include vitamin A and analogues thereof such as retinol and retinoic acid, carotenoids such as α-carotene and β-carotene, vitamin B₁ and analogues thereof such as thiamines, vitamin B₂ and analogues thereof such as riboflavin, vitamin B₆ and analogues thereof such as pyridoxine, vitamin B₁₂ and analogues thereof such as cyanocobalamin, folic acid, nicotinic acid, pantothenic acid, vitamin C and analogues thereof such as L-ascorbic acid, vitamin D and analogues thereof such as ergocalciferol and cholecalciferol, vitamin E and analogues thereof such as d-α-tocopherol and γ-tocopherol, Coenzyme Q10, vitamin K and analogues thereof, carnitine, ferulic acid, α-lipoic acid, orotic acid, and mixtures thereof.

In one specific embodiment, the vitamins are selected from hydrosoluble vitamins, such as vitamin C, vitamin B1, vitamin B2, vitamin B3, vitamin B5, vitamin B6, vitamin B7, vitamin B9, vitamin B12, carnitine and/or mixtures thereof.

Free Radical Scavengers, Anti-Atmospheric Pollution Agents, and Reactive Carbonyl Species Scavengers

Exemplary free radical scavengers and/or anti-atmospheric pollution agents, and/or reactive carbonyl species scavengers include tea extract, olive leaf extract, extract of Rosmarinus officinalis or extract of Eichhornia crassipes, benzopyrenes, vitamin C and derivatives thereof, vitamin E and derivatives thereof, in particular tocopheryl acetate, ascorbyl glycoside, phenols and polyphenols, in particular tannins, tannic acid and ellagic acid, gallocatechol, anthocyanins, chlorogenic acid, stilbenes, indoles, cysteine-containing amino acid derivatives, in particular N-acetylcysteine, ergothioneine, S-carboxymethylcysteine, chelating agents, in particular ethylene diamine tetraacetic acid (EDTA) trisodium ethylenediamine hydroxyethyl triacetate, sodium citrate, gluconic acid, phytic acid, sodium polyphosphate, sodium metaphosphate and ethylenediamines, carotenoids, bioflavonoids, ubiquinone, idebenone, catalase, superoxide dismutase, lactoperoxidase, glutathione peroxidase, glutathione, benzylidene camphor, pidolates, lignans, melatonin, oryzanol, carnosine and derivatives thereof, GHK [INCI: tripeptide-1] and its salts and/or derivatives, Aldenine® [INCI: hydrolyzed wheat protein, hydrolyzed soy protein, tripeptide-1], Preventhelia® [INCI: diaminopropionoyl tripeptide-33], diaminopropionyl-alanyl-asparaginyl-histidine, and Lipochroman™-6 [INCI: dimethylmethoxy chromanol] marketed by Lipotec, and mixtures thereof.

Hydrophilic Cosmetic, Pharmaceutical and Alimentary Active Agents

Examples of hydrophilic cosmetic, pharmaceutical and/or alimentary active agents include amino acids, peptides, proteins, hydrolyzed proteins, enzymes, hormones, vitamins, mineral salts, sugars, nucleotides, nucleic acids, molecules and extracts of biological and biotechnological origin, vaccines, synthetic or partially synthetic hydrophilic molecules and/or mixtures thereof.

Exemplary proteins, hydrolyzed protein, enzymes and hormones, as well as the commercial mixtures which contain them, include Elhibin® [INCI: glycine soja (soybean) protein], Preregen® [INCI: glycine soja (soybean) protein, oxidoreductases] and Regu®-Age [INCI: hydrolyzed rice bran protein, glycine soja (soybean) protein, oxidoreductases] marketed by Pentapharm/DSM, cadherins, integrins, selectins, hyaluronic acid receptors, immunoglobulins, fibroblast growth factor, connective tissue growth factor, platelet-derived growth factor, vascular endothelial growth factor, epidermal growth factor, insulin-like growth factor, keratinocyte growth factors, colony-stimulating growth factors, transforming growth factor-beta, tumor necrosis factor-alpha, interferons, interleukins, matrix metalloproteinases, receptor protein tyrosine phosphatases, hydrolyzed vegetable proteins such as hydrolyzed wheat protein, hydrolyzed soy protein or hydrolyzed whey protein, hydrolyzed vegetable protein, Collalift® [INCI: hydrolyzed malt extract] marketed by Coletica/Engelhard, Colhibin PF® [INCI: hydrolyzed rice protein] marketed by Pentapharm, Cytokinol® LS [INCI: hydrolyzed casein, hydrolyzed yeast protein, lysine HCL] marketed by Laboratoires Serobiologiques/Cognis, Liftline® [INCI: hydrolyzed wheat protein] and Ridulisse C® [hydrolyzed soy protein] marketed by Silab, catalase, superoxide dismutase, lactoperoxidase, glutathione peroxidase, lactoprotein, casein, lactoperoxidase, lysozyme, glycosidases, stratum corneum chymotryptic enzyme (SCCE); proteases such as trypsin, chymotrypsin, sutilain, papain and bromelain; DNA repair enzymes such as photolyase or T4 endonuclease V, lipase, luteinizing hormone (LH), follicle-stimulating hormone (FSH), growth hormone, insulin, and mixtures thereof.

Exemplary extracts of biological or biotechnological origin, which can be chemically modified, as well as the commercial mixtures which contain them, include vegetable extracts, marine extracts, cell extracts and extracts produced by microorganisms.

Exemplary vegetable extracts include hydrosoluble vegetable extracts, for example hydrosoluble extracts of chamomile, ivy, lemon, ginseng, raspberry, Roast amaranth, Rehmanniae radix, gardenia, carrot, orange, peach, pineapple, gentian, hibiscus flower, walnut leaf, pumpkin, peony, quinoa, boldo, rough bindweed, salvia, pomegranate, oregano, ginger, marjoram, cranberry, grape, tomato, green tea, black tea, aloe vera (Aloe Barbadensis), Sophora japonica, papaya, pineapple, pumpkin, sweet potato, Bupleurum chinensis, Cecropia obtusifolia, Celosia cristata, Centella asiatica, Chenopodium quinoa, Chrysanthellum indicum, Citrus aurantium amara, Coffea arabica, Coleus Forskohlii, Commiphora myrrha, Crithmum maritimum, Eugenia caryophyllus, Ginkgo biloba, Hedera helix (ivy), Hibiscus sabdariffa, Ilex paraguariensis, Laminaria digitata, Nelumbium speciosum, Paullinia cupana, Peumus boldus, Phyllacantha fibrosa, Prunella vulgaris, Prunus amygdalus dulcis, Ruscus aculeatus (Butcher's broom extract), Sambucus nigra, Spirulina platensis Algae, Uncaria tomentosa, Verbena Officinalis, Opuntia ficus-indica, Salix alba, Lupinus spp., Secale cereale, Tussilago farfara, Achillea millefolium, Azadirachta indica, Osmunda japonica, Artocarpus incisus, Bidens pilosa, Broussonetia papyrifera, Chlorella vulgaris, Cimicifuga racemosa, Emblica officinalis, Glycyrrhiza glabra, Glycyrrhiza uralensis, Ilex purpurea, Ligusticum lucidum, Ligusticum wallichii, Mitracarpus scaber, Morinda citrifolia, Morus alba, Morus bombycis, Naringi crenulata, Prunus domestica, Radix pseudostellaria, Rumex crispus, Rumex occidentalis, Sapindus mukorossi, Saxifraga sarmentosa, Scutellaria Galericulata, Sedum sarmentosum Bunge, Stellaria medica, Triticum Vulgare, Uva ursi, Withania somnifera, Aristolochia clematis, Rosa moschata, Echinacea angustifolia, Symphytum officinale, Equisetum arvense, Hypericum perforatum, Mimosa tenuiflora, Persea gratissima, Prunus africana, Tormentilla erecta, Solanum tuberosum, Rosmarinus officinalis, Vaccinium angustifolium, Macrocystis pyrifera algae, Padina pavonica, Malpighia puniciftolia, Cynara scolymus, Gossypium herbaceum, Panicum miliaceum, Morus nigra, Sesamum indicum, Glycine soja, Triticum vulgare, Glycine Max (soy), malt, flax, red clover, kakkon-to, white lupine, hazelnut, maize, beech tree shoots, Trifolium pratense (red clover), Phormium tenax (New Zealand flax), Cinnamomum verum, Laminaria saccharina, Spiraea ulmaria, Nettle Root, Pygeum africanum, Avena sativa, Arnica montana, Cinchona succirubra, Eugenia caryophyllata, Humulus lupulus, Hypericum perforatum, Mentha piperita, Rosmarinus officinalis, Thymus vulgaris, plant extract of the genus Silybum, extract of legume seeds, extracts of red algae from the genus Porphyra, Phytovityl C® [INCI: water, Zea Mays extract] marketed by Solabia, Micromerol™ [INCI: Pyrus Malus extract] and heather extract [INCI: Calluna vulgaris extract] marketed by Coletica/Engelhard/BASF, Proteasyl® TP LS8657 [INCI: Pisum sativum extract] marketed by Laboratoires Sérobiologiques/Cognis, Radicaptol™ [INCI: propylene glycol, water, Passiflora incarnata Flower extract, Ribes nigrum (blackcurrant) leaf extract, Vitis vinifera (grape) leaf extract] marketed by Solabia and ViaPure™ Boswellia [INCI: olibanum (Boswellia serrata) extract] marketed by Soliance, EquiStat™ [INCI Pyrus malus fruit extract, glycine soja seed extract] marketed by Coletica/Engelhard, Litchiderm™ [INCI: Litchi chinensis pericarp extract] and Arganyl™ [INCI: Argania spinosa leaf extract] marketed by Laboratories Sérobiologiques/Cognis, Dakaline™ [INCI: Prunus amygdalus dulcis, Anogeissus leiocarpus bark extract] marketed by Soliance, Actimp 1.9.3® [INCI: hydrolyzed lupine protein] marketed by Expanscience® Laboratoires, Pronalen® Firming HSC [INCI: Triticum vulgare, Silybum marianum, glycine soy, Equisetum arvense, Alchemilla vulgaris, Medicago sativa, Raphanus sativus] and Polyplant® Firming [INCI: coneflower, Centella asiatica, fucus, fenugreek] marketed by Provital, Lanablue® [INCI: sorbitol, algae extract] marketed by Atrium Innovations, Firmiderm® LS 9120 [INCI: Terminalia catappa leaf extract, Sambucus nigra flower extract, PVP, tannic acid] marketed by Laboratoires Serobiologiques/Cognis, among others.

The amount of hydrophilic active ingredient contained in the face mask or body patch may be from 0.00001 to 50 wt. % of the total weight of the mask (on an unhydrated basis), such as at least 0.0001 wt. %, or at least 0.001 wt. %, or at least 0.01 wt. %, and may be up to 40 wt. %, or up to 30 wt. %, or up to 10 wt. %.

Agents Inhibiting Elastin Degradation

Exemplary agents inhibiting elastin degradation include Elhibin® [INCI: glycine soja (Soybean) protein], Preregen® [INCI: glycine soja (soybean) protein, oxidoreductases] or Regu®-Age [INCI: hydrolyzed rice bran protein, glycine soja (Soybean) protein, oxidoreductases] marketed by Pentapharm/DSM, Juvenesce [INCI: ethoxydiglicol and caprylic triglyceride, retinol, ursolic acid, phytonadione, ilomastat], Micromerol™ [INCI: Pyrus Malus extract], heather extract [INCI: Calluna vulgaris extract], Extracellium® [INCI: hydrolyzed potato protein] or Flavagrum™ PEG [INCI: PEG-6 isostearate, hesperetin laurate] marketed by Coletica/Engelhard/BASF, Proteasyl® TP LS 8657 [INCI: Pisum sativum extract] marketed by Laboratoires Sérobiologiques/Cognis, Relistase™ [INCI: acetylarginyltriptophyl diphenylglycine] marketed by Lipotec, Sepilift™ DPHP [INCI: dipalmitoyl hydroxyproline] marketed by Seppic, Vitaderm® [INCI: alcohol, water, glycerin, hydrolyzed rice protein, Ilex aquifolium extract, sodium ursolate, sodium oleanolate] marketed by Rahn, Gatuline® Age Defense 2 [INCI: Juglans regia (walnut) seed extract] marketed by Gattefossé, IP 2000 [INCI: dextran, trifluoroacetyl tripeptide-2] marketed by IEB and Atrium, Radicaptol™ [INCI: propylene glycol, water, Passiflora incarnata flower extract, Ribes nigrum (blackcurrant) leaf extract, Vitis vinifera (grape) leaf extract] marketed by Solabia or ViaPure™ Boswellia [INCI: olibanum (Boswellia serrata) extract] marketed by Soliance, and mixtures thereof.

### Agents Stimulating Dermal or Epidermal Macromolecular Synthesis

Exemplary agents stimulating dermal or epidermal macromolecular synthesis include agents stimulating collagen synthesis, agents stimulating elastin synthesis, agents stimulating decorin synthesis, agents stimulating laminin synthesis, agents stimulating chaperone synthesis, agents stimulating sirtuin synthesis, agents stimulating hyaluronic acid synthesis, agents stimulating aquaporin synthesis, agents stimulating fibronectin synthesis, agents inhibiting collagen degradation, agents inhibiting elastin degradation, agents inhibiting serine proteases such as leukocyte elastase or cathepsin G, agents stimulating fibroblast proliferation, agents stimulating adipocyte proliferation, agents stimulating adipocyte differentiation, agents stimulating angiogenesis, agents stimulating glycosaminoglycan synthesis, DNA repair agents and/or DNA protecting agents, for example extracts of Centella asiatica, Saccharomyces cerevisiae, Solanum tuberosum, Rosmarinus officinalis, Vaccinium angustifolium, extract of the algae Macrocystis pyrifera, Padina pavonica, extract of the plants soy, malt, flax, sage, red clover, kakkon-to, white lupine, hazelnut extract, corn extract, yeast extract, extract of beech tree shoots, extract of leguminosae seeds, extract of plant hormones such as gibberellins, auxins or cytokinins among others, or extract of zooplankton Salina, the product of milk fermentation with Lactobacillus Bulgaricus, asiaticosides and derivatives thereof, vitamin C and derivatives thereof, cinnamic acid and derivatives thereof, Matrixyl®[INCI: palmitoyl pentapeptide-3], Matrixyl® 3000 [INCI: palmitoyl tetrapeptide-3, palmitoyl oligopeptide] or Biopeptide CL™ [INCI: glyceryl polymethacrylate, propylene glycol, palmitoyl oligopeptide] marketed by Sederma, Antarcticine® [INCI: Pseudoalteromonas ferment extract], Decorinyl® [INCI: tripeptide-10 citrulline], Serilesine® [INCI: hexapeptide-10], Lipeptide [INCI: hydrolyzed vegetable protein], Aldenine® [INCI: hydrolyzed wheat protein, hydrolyzed soy protein, tripeptide-1], Peptide AC29™ [INCI: acetyl tripeptide-30 citrulline], acetyl-arginyl-phenylglycyl-tryptophyl-phenylglycine, acetyl-arginyl-phenylglycyl-valyl-glycine, or acetyl-arginyl-phenylglycyl-valyl-phenylglycine, marketed by Lipotec, Drieline® PF [INCI: yeast betaglucan] marketed by Alban Muller, Phytovityl C^{°} [INCI: water, Zea Mays extract] marketed by Solabia, Collalift® [INCI: hydrolyzed malt extract] marketed by Coletica/Engelhard, Phytocohesine® PSP [proposed INCI: sodium beta-sitosteryl sulfate] marketed by Seporga, minerals such as calcium among others, retinoids and derivatives thereof, isoflavonoids, carotenoids, in particular lycopene, pseudodipeptides, retinoids and derivatives thereof such as retinol and retinyl palmitate, heparinoids, and mixtures thereof.

Matrix Metalloproteinase-Inhibiting Agents

Exemplary matrix metalloproteinase-inhibiting agents include ursolic acid, isoflavones such as genistein, quercetin, carotenoids, lycopene, soy extract, cranberry extract, rosemary extract, Trifolium pratense (red clover) extract, Phormium tenax (New Zealand flax) extract, kakkon-to extract, sage extract, retinol and derivatives thereof, retinoic acid and derivatives thereof, sapogenins such as diosgenin, hecogenin, smilagenin, sarsapogenin, tigogenin, yamogenin and yuccagenin, Collalift® [INCI: hydrolyzed malt extract], Juvenesce [INCI: ethoxydiglicol and caprylic triglyceride, retinol, ursolic acid, phytonadione, ilomastat] and EquiStat™ [INCI Pyrus malus fruit extract, glycine soja seed extract] marketed by Coletica/Engelhard, Pepha®-TIMP [INCI: human oligopeptide-20], Regu®-Age [INCI: hydrolyzed rice bran protein, glycine soja protein, oxidoreductases] and Colhibin™ [INCI: hydrolyzed rice protein] marketed by Pentapharm, Lipeptide [INCI: hydrolyzed vegetable protein], Peptide AC29 [INCI: acetyl tripeptide-30 citrulline], and acetyl-arginyl-asparaginyl-histidyl-citrulline-amide marketed by Lipotec, Litchiderm™ [INCI: Litchi chinensis pericarp extract] and Arganyl™ [INCI: Argania spinosa leaf extract] marketed by Laboratories Sérobiologiques/Cognis, MDI Complex® [INCI: glycosaminoglycans] and ECM-Protect® [INCI: water, dextran, tripeptide-2] marketed by Atrium Innovations, Dakaline™ [INCI: Prunus amygdalus dulcis, Anogeissus leiocarpus bark extract] marketed by Soliance, Homeostatine™ [INCI: Enteromorpha compressa, Caesalpinia spinosa] marketed by Provital, TIMP-Peptide™ [proposed INCI: acetyl hexapeptide] and ECM Moduline™ [proposed INCI: palmitoyl tripeptide] marketed by Infinitec Activos, IP2000 [INCI: dextran, trifluoroacetyl tripeptide-2] marketed by Institut Européen de Biologie Cellulaire, Actimp 1.9.3® [INCI: hydrolyzed lupine protein] marketed by Expanscience Laboratories, Vitaderm® [INCI: alcohol, water, glycerin, hydrolyzed rice protein, ilex aquifolium extract, sodium ursolate, sodium oleanolate] marketed by Rahn, adapalene, tetracyclines and derivatives thereof such as minocycline, rolitetracycline, chlortetracycline, metacycline, oxytetracycline, doxycycline, demeclocycline and their salts, Batimastat [BB94; [4-(N-hydroxyamino)-2R-isobutyl-3S-(thiophene-2-ylthiomethyl) succinyl]-L-phenylalanine-N-methylamide], Marimastat™ [BB2516; [2S—[N4(R*),2R*,3S]]—N4[2,2-dimethyl-1-[methylaminocarbonyl]propyl]-N1,2-dihydroxy-3-(2-methylpropyl)butanediamide], and mixtures thereof.

### Firming, Redensifying, and Restructuring Agents

Exemplary firming and/or redensifying and/or restructuring agents include extracts of Malpighia puniciftolia, Cynara scolymus, Gossypium herbaceum, Aloe Barbadensis, Panicum miliaceum, Morus nigra, Sesamum indicum, Glycine soja, Triticum vulgare, Pronalen® Firming HSC [INCI: Triticum vulgare, Silybum marianum, glycine soy, Equisetum arvense, Alchemilla vulgaris, Medicago sativa, Raphanus sativus] and Polyplant® Firming [INCI: Coneflower, Centella Asiatica, Fucus, Fenugreek] marketed by Provital, Lanablue® [INCI: sorbitol, algae extract] marketed by Atrium Innovations, Pepha®-Nutrix [INCI: natural nutrition factor] marketed by Pentapharm, vegetable extracts which contain isoflavones, Biopeptide EL™ [INCI: palmitoyl oligopeptide], Biopeptide CL™ [INCI: palmitoyl oligopeptide], Vexel® [INCI: water, propylene glycol, lecithin, caffeine, palmitoyl carnitine], Matrixyl® [INCI: palmitoyl pentapeptide-3], Matrixyl® 3000 [INCI: palmitoyl tetrapeptide-3, palmitoyl oligopeptide] and Bio-Bustyl™ [INCI: glyceryl polymethacrylate, Rahnella soy protein ferment, water, propylene glycol, glycerin, PEG-8, palmitoyl oligopeptide] marketed by Sederma, Dermosaccharides® HC [INCI: glycerin, water, glycosaminoglycans, glycogen], Aglycal® [INCI: mannitol, cyclodextrin, glycogen, Arctostaphylos uva ursi leaf extract], Cytokinol® LS [INCI: hydrolyzed casein, hydrolyzed yeast protein, lysine HCl] and Firmiderm® LS 9120 [INCI: Terminalia catappa leaf extract, Sambucus Nigra Flower extract, PVP, tannic acid] marketed by Laboratoires Serobiologiques/Cognis, Liftline® [INCI: hydrolyzed wheat protein], Raffermine® [INCI: hydrolyzed soy flour] and Ridulisse C® [hydrolyzed soy protein] marketed by Silab, Serilesine® [INCI: hexapeptide-10], Decorinyl™ [INCI: tripeptide-10 citrulline], Trylagen® [INCI: Pseudoalteromonas ferment extract, hydrolyzed wheat protein, hydrolyzed soy protein, tripeptide-10 citrulline, tripeptide-1], marketed by Lipotec, Ursolisome® [INCI: lecithin, ursolic acid, atelocollagen, xanthan gum, sodium chondroitin sulfate] and Collalift® [INCI: hydrolyzed malt extract] marketed by Coletica/Engelhard, Syn®-Coll [INCI: palmitoyl tripeptide-5] marketed by Pentapharm, Hydriame® [INCI: water, glycosaminoglycans, sclerotium gum] marketed by Atrium Innovations, IP2000 [INCI: dextran, trifluoroacetyl tripeptide-2] marketed by Institut Européen de Biologie Cellulaire, and mixtures thereof.

### Anti-Glycation Agents

Exemplary anti-glycation agents include Vaccinium angustifolium extracts, ergothioneine and derivatives thereof, lysine, Aldenine® [INCI: hydrolyzed wheat protein, hydrolyzed soy protein, tripeptide-1], Vilastene™ [INCI: lysine HCl, lecithin, tripeptide-10 citrulline], dGlyage™ [INCI: lysine HCl, lecithin, tripeptide-9 citrulline] and Eyeseryl® [INCI: acetyl tetrapeptide-5] marketed by Lipotec, hydroxystilbenes and derivatives thereof, resveratrol, 3,3′,5,5′-tetrahydroxystilbene, and mixtures thereof.

### 5α-Reductase Inhibiting Agents

Exemplary 5α-reductase inhibiting agents include extracts of Cinnamomum verum, Laminaria saccharina, Spiraea ulmaria, Nettle Root, Pygeum africanum, Avena Sativa, Serenoa repens, extracts of the plants Arnica montana, Cinchona succirubra, Eugenia caryophyllata, Humulus lupulus, Hypericum perforatum, Mentha piperita, Rosmarinus officinalis, Salvia officinalis, and Thymus vulgaris, extract of plants of the genus Silybum, extracts of plants which contain sapogenins and in particular extract of plants of the genus Dioscorea, phytosterols, retinoids and in particular retinol, sulfur and derivatives thereof, zinc salts and in particular zinc lactate, zinc gluconate, zinc pidolate, zinc carboxylate, zinc salicylate and zinc cysteate, selenium chloride, vitamin B6, pyridoxine, capryloyl glycine, sarcosine, finasteride, dutasteride, izonsteride, turosteride and their salts, and mixtures thereof.

Lysyl- and/or Prolvl-Hydroxvlase-Inhibiting Agents

Exemplary lysyl- and/or prolyl-hydroxylase-inhibiting agents include 2,4-diaminopyrimidine 3-oxide, 2,4-diamino-6-piperidinopyrimidine 3-oxide, and mixtures thereof.

### Defensin Synthesis-Stimulating Agents

Exemplary defensin synthesis-stimulating agents include extracts of or hydrolyzed Aloe Vera, Roast amaranth, Rehmanniae radix, arnica, gardenia, carrot, orange, peach, pineapple, mint, gentian, hibiscus flower, walnut tree leaf, calabaza, peony, quinoa, boldo, rough bindweed, sunflower, elderberry, seaweed, hydrolyzed corn, hydrolyzed soy, hydrolyzed rice, valine and its isomers and derivatives, calcium and its salts, α-MSH and fragments contained in the amino acid sequence of α-MSH, vitamin A and its derivatives and precursors, vitamin D3 and its derivatives, jasmonic acid, fumaric acid, malic acid, citric acid, ascorbic acid, lactic acid, acetic acid, adipic acid, tartaric acid, cinnamic acid, glutamic acid, succinic acid, inulin, alkyl glucosides, poly-D-glutamic acid, glycine, L-methionine, L-alanine, L-citrulline, lactoprotein, casein, lactoperoxidase, lysozyme, polyphenol, alkyl glucosides, Lactobacillus extract, fusobacteria extracts, non-photosynthetic and non-fruiting filamentous bacteria, Bodyfensine™ [INCI: acetyl dipeptide-3 aminohexanoate] marketed by Lipotec, and mixtures thereof.

### Antiseptic Agents and Disinfectants

Exemplary antiseptic agents and disinfectants include those serving as bactericidal, bacteriostatic, antimicrobial, germicidal, fungicidal, fungistatic and/or germ inhibiting agents.

Examples of such agents include, macrolides, pyranosides, calcium channel blockers, for example cinnarizine and diltiazem; hormones, for example estril and analogues thereof, thyroxine and/or its salts, caprylyl glycol, imidazolidinyl urea, sodium 4-oxybenzoate methyl, methyl 4-hydroxybenzoate [INCI: methylparaben], ethyl 4-oxybenzoate, ethyl 4-hydroxybenzoate [INCI: ethylparaben], propyl 4-oxybenzoate, isopropyl 4-oxybenzoate, propyl 4-hydroxybenzoate [INCI: propylparaben], butyl 4-oxybenzoate, butyl 4-hydroxybenzoate [INCI: butylparaben], isobutyl 4-hydroxybenzoate [INCI: isobutylparaben], 1,3-bis(hydroxymethyl)-5,5-dimethylimidazolidine-2,4-dione [INCI: DMDM hydantoin], benzyl 4-oxybenzoate, benzyl 4-hydroxybenzoate [INCI: benzylparaben], benzyl alcohol, dehydroacetic acid, benzoic acid, sodium benzoate, potassium sorbate, dehydroacetic acid, sodium dehydroacetate sorbic acid, salicylic acid, formic acid, propionic acid, 2-bromo-2-nitropropane-1,3-diol, 3-p-chlorophenoxy-1,2-propanediol [INCI: chlorphenesin], dichlorobenzyl alcohol, iodopropynyl butylcarbamate, benzalkonium chloride, odor-absorbing fungicides such as zinc ricinoleate, cyclodextrins, benzethonium chloride, chlorhexidine, ethanol, propanol, 1,3-butanediol, 1,2-propylene glycol, undecylenic acid, dehydroacetic acid, N-methylmorpholine acetonitrile (MMA), isopropanol, methanol, 1,2-hexanediol, 1,2-octanediol, pentylene glycol, glycerin laurate, glycerin caprylate, glyceryl caprate, benzoyl peroxide, chlorhexidine gluconate, triclosan and derivatives thereof, phenoxyethanol, terpinen-4-ol, α-terpineol, resorcinol, stiemycin, erythromycin, neomycin, clindamycin and its esters, tetracyclines, metronidazole, azelaic acid, tolnaftate, nystatin, clotrimazole, ketoconazole, derivatives of zinc such as zinc pyrithionate or trithionate, zinc oxide and zinc undecylenate, piroctone olamine, isothiazolinones, selenium sulfur, benzyl hemiformal, boric acid, sodium borate, 6,6-dibromo-4,4-dichloro-2,2′-methyl ene-diphenol [INCI: bromochlorophene], 5-bromo-5-nitro-1,3-dioxane, tosylchloramide sodium [INCI: chloramine T], chloroacetamide, p-chloro-m-cresol, 2-benzyl-4-chlorophenol [INCI: chlorophene], dimethyl oxazolidine, dodecyl dimethyl-2-phenoxyethyl ammonium bromide [INCI: domiphen bromide], 7-ethyl bicyclo-oxazolidine, hexetidine, glutaraldehyde, N-(4-chlorophenyl)-N-[4-chloro-3-(trifluoromethyl)phenyl]-urea [INCI: cloflucarban], 2-hydroxy-4-isopropyl-2,4,6-cycloheptatriene-1-one [INCI: Hinokitiol], isopropylmethylphenol, mercury salts, aluminum salts, nisin, phenoxyisopropanol, o-phenylphenol, 3-heptyl-2-[(3-heptyl-4-methyl-3H-thiazole-2-ylidene)methyl]-4-methylthi azole iodide [INCI: Quaternium-73], silver chloride, sodium iodide, thymol, undecylenic acid, diethylenetriaminepentaacetic acid, ethyl enediaminetetraacetic acid and ethyl enediaminetetraacetates, lactoperoxidase, glucose oxidase, lactoferrin, alkylaryl sulfonates, halogenated phenols, phenol mercury acetate and/or mixtures thereof, benzamidines, isothiazolines, derivatives of phthalimide, derivatives of pyridine, guanidines, quinolines, 1,2-dibromo-2,4-dicyanobutane, iodine-2-propylbutyl carbamate, iodine, tamed iodines, peroxo compounds, 4-chloro-3,5-dimethylphenol, 2,2′-methylene-bis(6-bromo-4-chlorophenol), 3-methyl-4-(1-methyl ethyl)phenol, 3-(4-chlorophenoxy)-1,2-propanediol, 3,4,4′-trichlorocarbanilide (TTC), beta-lactams, thiamine essence, eugenol, farnesol, glycerol monolaurate, diglycerin monocaprinate, N-alkyl salicylic acid amides such as n-octyl salicylic acid amide or n-decyl salicylic acid amide, derivatives of halogenated xylene and cresol, such as p-chloro-meta-cresol or p-chloro-meta-xylene, extracts of Allium sativum, Calendula officinalis, Chamomilla recutita, Echinacea purpurea, Hyssopus officinalis, Melaleuca alternifolia or tea tree oil, carnation essence, menthol and mint essence, light sensitive dye No. 101, light sensitive dye No. 201 and light sensitive dye No. 401, and mixtures thereof.

### NO-Synthase-Inhibiting Agents

Exemplary NO-synthase-inhibiting agents include extracts of the plants Vitis vinifera, Olea europaea, Gingko biloba, and mixtures thereof.

### Desquamating Agents and Keratolytic Agents

Exemplary desquamating agents and/or keratolytic agents and/or exfoliating agents include hydroxy acids and derivatives thereof, β-hydroxyacids, in particular salicylic acid and derivatives thereof, and gentisic acid; α-hydroxyacids and its salts, such as glycolic acid, ammonium glycolate, lactic acid, 2-hydroxyoctanoic acid, α-hydroxycaprylic acid, mandelic acid, citric acid, malic acid and tartaric acid; α- and β-hydroxybutyric acids; polyhydroxy acids such as gluconic acid, glucuronic acid and saccharic acid; keto acids such as pyruvic acid, and glyoxylic acid; pyrrolidinecarboxylic acid; cysteic acid and derivatives thereof; aldobionic acids; azelaic acid and derivatives thereof such as azeloyl diglycinate; ascorbic acid and derivatives thereof such as 6-O-palmitoylascorbic acid, ascorbyl glucoside, dipalmitoyl ascorbic acid, magnesium salt of ascorbic acid-2-phosphate (MAP), sodium salt of ascorbic acid-2-phosphate (NAP), ascorbyl tetraisopalmitate (VCIP); nicotinic acid, its esters and nicotinamide (also called vitamin B3 or vitamin PP); nordihydroguaiaretic acid; urea; oligofucoses; cinnamic acid; derivatives of jasmonic acid; hydroxy stilbenes such as resveratrol; Saccharum officinarum extract; enzymes involved in desquamation or degradation of the corneodesmosomes, such as glycosidases, stratum corneum chymotryptic enzyme (SCCE) and other proteases such as trypsin, chymotrypsin, sutilain, papain and bromelain; chelating agents such as ethylenediaminetetraacetic acid (EDTA) and salts thereof, aminosulfonic compounds such as 4-(2-hydroxyethyl)piperazine-1-ethanesulfonic acid (HEPES) and sodium methylglycine diacetate (TRILON® M marketed by BASF); derivatives of 2-oxothiazolidine-4-carboxylic acid (procysteine); derivatives of sugars such as O-octanoyl-6-D-maltose and N-acetylglucosamine; chestnut extract (Castanea sativa) such as that marketed by SILAB under the name Recoverine® [INCI: water, Castanea sativa seed extract]; opuntia extract (Opuntia ficus-indica) such as that marketed by SILAB as Exfolactive® [INCI: hydrolyzed Opuntiaficus Indica flower extract]; Phytosphingosine SLC® [INCI: salicyloyl phytosphingosine] marketed by Degussa/Evonik, Peel-Moist™ [INCI: glycerin, papain, calcium pantothenate, xanthan gum, caprylyl glycol, urea, magnesium lactate, ethylhexylglycerin, potassium lactate, serine, alanine, proline, magnesium chloride, sodium citrate]; extract or combination of extracts of Sophora japonica, papaya, pineapple, pumpkin or sweet potato, and mixtures thereof.

Melanin Stimulating, Propigmenting, Self-Tanning and/or Melanocyte Proliferation Stimulating Agents

Example agents which stimulate the synthesis of melanin, the propigmenting agent, the self-tanning agent and/or the melanocyte proliferation stimulating agent include extracts of Citrus Aurantium Dulcis Fruit, Coleus forskohlii, Coleus esquirolii, Coleus scutellarioides, Coleus xanthanthus, Ballota nigra, Ballota lanata, Ballota suaveolens, Marrubium cylleneum, Cistus creticus, Amphiachyris amoena, Aster oharai, Otostegia fruticosa, Plectranthus barbatus, Haliniur viscosum and Larix laricina, dihydroxyacetone and derivatives thereof, sugars, for example erythrulose, melanin and derivatives thereof including melanin polymers and derivatives of melanin with a low molecular weight which are soluble in water, forskolin and derivatives thereof including deacetylforskolin and isoforskolin, tyrosine and derivatives thereof including acetyl tyrosine, oleoyl tyrosine, 3-amino tyrosine and 3-nitrotyrosine, copper salts such as CuCl₂, carotenoids, canthaxanthins, polymers of dihydroxyindole carboxylic acid, 3,4-dihydroxybenzoic acid, 3-amino-4-hydroxybenzoic acid, aloin, emodin, alizarin, dihydroxyphenylalanine, 4,5-dihydroxynaphthalene-2-sulfonic acid, 3-dimethylaminophenol and p-aminobenzoic acid, Melatime™ [INCI: acetyl tripeptide-40] marketed by Lipotec, Heliostatine ISR™ [INCI: water, glycerin, Pisum sativum extract] marketed by Vincience/ISP, Vegetan® [INCI: dihydroxyacetone] or Vegetan® Premium [INCI: dihydroxyacetone, melanin] marketed by Soliance, MelanoBronze™ [INCI: Vitex agnus-castus extract, acetyl tyrosine] marketed by Mibelle Biochemistry, Melitane® [INCI: acetyl hexapeptide-1] marketed by Institut Européen de Biologie Cellulaire/Unipex Innovations, Actibronze® [INCI: hydrolyzed wheat protein, acetyl tyrosine, copper gluconate] and Instabronze® [INCI: dihydroxyacetone, tyrosine] marketed by Alban Muller, Thalitan™ [INCI: hydrolyzed algin, magnesium sulfate, manganese sulfate] marketed by CODIF, Tyrosilane® [INCI: methylsilanol acetyl tyrosine] marketed by Exsymol, Tyr-Excel™ [INCI: oleoyl tyrosine, Luffa Cylindrica seed oil, oleic acid] or Tyr-Ol™ [INCI: oleoyl tyrosine, butylene glycol, oleic acid] marketed by Sederma/Croda, Bronzing S.F. [proposed INCI: butyryl pentapeptide] marketed by Infinitec Activos or Biotanning® [INCI: hydrolyzed Citrus aurantium dulcis fruit extract] marketed by Silab, and mixtures thereof.

Lipolytic Agents, Agents Stimulating Lipolysis, Venotonic Agents and Anti-Cellulite Agents

Exemplary lipolytic agents, agents stimulating lipolysis, venotonic agents and/or anti-cellulite agents include extracts of Bupleurum chinensis, Cecropia obtusifolia, Celosia cristata, Centella asiatica, Chenopodium quinoa, Chrysanthellum indicum, Citrus aurantium amara, Coffea arabica, Coleus forskohlii, Commiphora myrrha, Crithmum maritimum, Eugenia caryophyllus, Ginkgo biloba, Hedera helix (ivy extract), Hibiscus sabdariffa, Ilex paraguariensis, Laminaria digitata, Nelumbium speciosum, Paullinia cupana, Peumus boldus, Phyllacantha fibrosa, Prunella vulgaris, Prunus amygdalus dulcis, Ruscus aculeatus (Butcher's broom extract), Sambucus nigra, Spirulina platensis algae, Uncaria tomentosa and Verbena officinalis, dihydromyricetin, coenzyme A, lipase, glaucine, visnadine, Regu®-Shape [INCI: isomerized linoleic acid, lecithin, glycerin, polysorbate 80] marketed by Pentapharm/DSM, UCPeptide™ V [INCI: pentapeptide] and AT Peptide™ IS [INCI: tripeptide-3] marketed by Vincience/ISP, Liporeductyl®[INCI: caffeine, Butcher's broom (Ruscus aculeatus) root extract, TEA-hydroiodide, carnitine, ivy (Hedera helix) extract, escin, tripeptide-1] marketed by Lipotec, Adiposlim™ [INCI: sorbitan laurate, lauroyl proline] marketed by SEPPIC, caffeine, carnitine, escin, triethanolamine iodide, and mixtures thereof.

Heat Shock Protein Synthesis Stimulating Agents

Exemplary heat shock protein synthesis stimulating agents include extracts of Opuntiaficus indica, Salix alba, Lupinus spp., Secale cereale, extracts of red algae from the genus Porphyra, extracts of crustaceans from the genus Artemia, jojoba seed oil, grape seed extracts, green tea extracts, geranylgeranylacetone, celastrol, zinc and its salts, 2-cyclopenten-1-one, proteasome inhibitors, for example bortezomib; prostaglandins and derivatives thereof, hydroxylamine and derivatives thereof, for example bimoclomol; chalcone and derivatives thereof, hyperosmotic agents, for example sorbitol and derivatives thereof, mannitol and derivatives thereof or glycerol and derivatives thereof, isosorbide (dianhydro-D-glucitol) urea or salicylic acid and derivatives thereof among others, Thermostressine™ [INCI: acetyl tetrapeptide-22], and mixtures thereof.

Agents Inhibiting Sweat-Degrading Enzymes

Exemplary agents for inhibiting sweat-degrading enzymes include trialkyl citrates such as trimethyl citrate, tripropyl citrate, triisopropyl citrate, tributyl citrate or triethyl citrate; lanosterine sulfate and lanosterine phosphate, cholesterol, campesterol, stigmasterol and sitosterol; dicarboxylic acids and their esters, such as glutaric acid, monoethyl glutarate, diethyl glutarate, adipic acid, monoethyl adipate, diethyl adipate; malonic acid and diethyl malonate, hydroxycarboxylic acids and their esters such as malic acid, tartaric acid and diethyl tartrate, zinc glycinate, and mixtures thereof.

Agents Stimulating or Regulating Keratinocyte Differentiation

Exemplary agents for stimulating or regulating keratinocyte differentiation include minerals such as calcium, retinoids such as retinol and tretinoin, analogues of vitamin D3 such as calcitriol, calcipotriol and tacalcitol, lupine (Lupinus albus) extract such as that marketed by SILAB under the name Structurin® [INCI: hydrolyzed lupine protein], β-sitosterol sulfate, such as that marketed by Vincience/ISP under the name Phytocohesine PSP® [INCI: sodium beta-sitosterol sulfate], maize (Zea Mays) extract such as that marketed by Solabia under the name Phytovityl C® [INCI: water, Zea Mays extract], Helix aspersa Miller glycoconjugates, and mixtures thereof.

Exopolysaccharides

Exemplary exopolysaccharides, such as those of bacterial origin, include those secreted by a strain of the Halomonas anticariensis species, which reduce lipid accumulation, as described in WO 2015/063240, and an exopolysaccharide which inhibits neuronal exocytosis and stimulates the fibroblast proliferation which is excreted by the strain of the Vibrio sp. species with deposit number CNCM 1-4239 according to the Budapest Treaty on Sep. 4, 2009, in the “Collection Nationale de Culture de Microorganismes” [National Microorganism Culture Collection](CNCM), Pasteur Institute, 28 rue du Docteur Roux, 75724 Paris, France, as described in WO 2014147255 and U.S. Ser. No. 14/778,874, filed Sep. 21, 2015.

Exemplary cell extracts and extracts produced by microorganisms, or commercial mixtures which contain them include hydrosoluble cell extracts and hydrosoluble extracts produced by microorganisms, for example Antarcticine® [INCI: Pseudoalteromonas ferment extract] and Trylagen® [INCI: Pseudoalteromonas ferment extract, hydrolyzed wheat protein, hydrolyzed soy protein, Tripeptide-10 citrulline, Tripeptide-1] marketed by Lipotec, yeast extract, extract of Saccharomyces cerevisiae and the product of milk fermentation with Lactobacillus Bulgaricus, among others.

Excipients

Excipients which may be present include emulsifiers, organic solvents, surfactants, liquid propellants, binders and thickeners, fillers, lubricants, glidants, pigments, dyes, perfumes, flavoring agents, preservatives, and combinations thereof.

Components serving as lubricants, solvents, propellants, binders and thickeners and emulsifiers may include one or more of liquid hydrocarbons, waxes, natural fats and fatty oils, alcohols, ethers, esters, silicone oils, monosaccharides, polymers, and the like.

Exemplary liquid hydrocarbons include α-olefins, C₁₀-C₄₀ alkanes, C₁₀-C₄₀ alkenes, and mixtures thereof, such as squalene, ceresin, mineral oils, and petroleum jelly.

Exemplary waxes include microcrystalline wax, natural waxes such as jojoba oil, carnauba wax, candelilla wax, rice bran wax, shellac, lanolin, mink sebaceous wax, spermaceti wax, sugarcane wax, sperm whale oil, beeswax and montan wax.

Exemplary natural fats and fatty oils include avocado oil, almond oil, olive oil, extra virgin olive oil, sesame seed oil, rice bran oil, rice oil, rice germ oil, corn oil, safflower oil, soybean oil, maize oil, rape seed oil, persic oil, palm kernel oil, palm oil, castor oil, sunflower oil, high oleic sunflower oil, grape seed oil, cottonseed oil, coconut oil, hydrogenated coconut oil, beef tallow, hydrogenated oil, horse oil, mink oil, yolk oil, yolk fat oil, rose hip oil, kukui nut oil, evening primrose oil, wheat germ oil, peanut oil, Camellia japonica oil, Camellia kissi oil, cacao butter, Japan wax, beef bone tallow, nest's-foot oil, swine tallow, equine tallow, ovine tallow, shea butter, macadamia nut oil and meadow foam seed oil.

Exemplary fatty acids include lauric acid, myristic acid, palmitic acid, stearic acid, behenic acid, oleic acid, linoleic acid, linolenic acid, γ-linolenic acid, isostearic acid, 12-hydroxystearic acid, undecenoic acid and coconut oil fatty acid.

Exemplary lower alcohols include ethanol, 1-propanol, 2-propanol, 1-butanol, 2-butanol and benzyl alcohol. Exemplary higher alcohols include isostearyl alcohol, 2-octyldodecan-1-ol, 2-hexyldecan-1-ol, cholesterol, phytosterols, lauryl alcohol, myristyl alcohol, cetyl alcohol, stearyl alcohol, oleyl alcohol, behenyl alcohol and cetostearyl alcohol. Exemplary polyhydric alcohols include ethylene glycol, diethylene glycol, triethylene glycol, polyethylene glycol, propylene glycol, dipropylene glycol, polypropylene glycol, pentanediol, glycerin, diglycerin, polyglycerin, isoprene glycol, 1,3-butylene glycol, 3-methyl-1,3-butanediol, 1,3-butanediol, 1,2-pentanediol and 1,2-hexanediol.

Exemplary alkyl glyceryl ethers include stearyl monoglyceride, 3-hexadecoxypropane-1,2-diol, 3-[(Z)-octadec-9-enoxy]propane-1,2-diol and isostearyl glyceryl ether.

Exemplary esters include isopropyl myristate, butyl myristate, isopropyl palmitate, ethyl stearate, butyl stearate, ethyl oleate, ethyl linoleate, isopropyl linoleate, cetyl caprylate, hexyl laurate, isooctyl myristate, decyl myristate, myristyl myristate, cetyl myristate, octadecyl myristate, cetyl palmitate, stearyl stearate, decyl oleate, oleyl oleate, cetyl ricinoleate, isostearyl laurate, isotridecyl myristate, isocetyl myristate, isostearyl myristate, 2-octyldodecyl myristate, 2-ethylhexyl palmitate, isocetyl palmitate, isostearyl palmitate, 2-ethylhexyl stearate, isocetyl stearate, isodecyl oleate, octyldodecyl oleate, octyldodecyl ricinoleate, ethyl isostearate, isopropyl isostearate, cetyl 2-ethylhexanoate, cetostearyl 2-ethylhexanoate, stearyl 2-ethylhexanoate, hexyl isostearate, ethylene glycol dioctanoate, ethylene glycol dioleate, propylene glycol dicaprylate, propylene glycol dicaprylate/dicaprate, lauryl lactate, myristyl lactate, cetyl lactate, trioctyl citrate, diisostearyl malate, 2-ethylhexyl hydroxystearate, diisopropyl adipate, diisopropyl sebacate, dioctyl sebacate, cholesteryl stearate, cholesteryl isostearate, cholesteryl hydroxystearate, cholesteryl oleate, dihydrocholesteryl oleate, phytosteryl isostearate, phytosteryl oleate, isocetyl 12-stearoyl hydroxystearate, stearyl 12-stearoyl hydroxystearate, isostearyl 12-stearoyl hydroxystearate, octyl isononanoate.

Exemplary silicone oils include polysiloxanes, polyether modified silicones, alcohol modified silicones, alkyl modified silicones, and amino modified silicones.

Exemplary saccharides include mannitol, sorbitol, xylitol, maltitol, erythritol, pentaerythritol, glucose, sucrose, fructose, lactose, maltose, xylose and trehalose.

Exemplary polymers include sodium alginate, carrageenan, agar, guar gums, tamarind gum, dextrin, starch, locust bean gum, gum arabic, pectin, quince, chitosan, starch, curdlan, xanthan gum, dextran, pullulan, microcrystalline cellulose, methyl cellulose, ethyl cellulose, hydroxyethyl cellulose, hydroxypropyl cellulose, hydroxypropyl methylcellulose, carboxymethyl cellulose, carboxy starch, cationized cellulose, starch phosphate ester, albumin, casein, gelatin, sodium polyacrylate, polyacrylamides, carboxyvinyl polymers, polyethylene imines, polyethylene glycol, polyvinyl alcohol, polyvinyl pyrrolidone, polyvinyl ether, polyacrylamides, acrylic acid copolymers, methacrylic acid copolymers, maleic acid copolymers, vinylpyridine copolymers, ethylene/acrylic acid copolymers, vinyl pyrrolidone based polymers, vinyl alcohol/vinyl pyrrolidone copolymers, N-substituted acrylamide based polymers, amino-modified silicones, dimethylacrylic acid based polymers, acrylic acid based anionic polymers, methacrylic acid based anionic polymers, modified silicone, acrylate/methacrylate C₁₀-C₃₀ alkyl copolymers, and polyoxyethylene/polyoxypropylene copolymers.

Exemplary anionic surfactants include potassium coconut oil fatty acid, sodium coconut oil fatty acid, triethanolamine coconut oil fatty acid, potassium laurate, sodium laurate, triethanolamine laurate, potassium myristate, sodium myristate, isopropanolamine myristate, potassium palmitate, sodium palmitate, isopropanolamine palmitate, potassium stearate, sodium stearate, triethanolamine stearate, potassium oleate, sodium oleate, castor oil fatty acid sodium, zinc undecylate, zinc laurate, zinc myristate, magnesium myristate, zinc palmitate, zinc stearate, calcium stearate, magnesium stearate, aluminum stearate, calcium myristate, magnesium myristate, aluminum dimyristate, aluminum isostearate, polyoxyethylene lauryl ether acetate, sodium polyoxyethylene lauryl ether acetate, polyoxyethylene tridecyl ether acetate, sodium polyoxyethylene tridecyl ether acetate, sodium stearoyl lactate, sodium isostearoyl lactate, sodium lauroyl sarcosinate, coconut oil fatty acid sarcosinate, sodium coconut oil fatty acid sarcosinate, coconut oil fatty acid sarcosine triethanolamine, lauroyl sarcosine, potassium lauroyl sarcosinate, lauroyl sarcosine triethanolamine, oleoyl sarcosine, sodium myristoyl sarcosinate, sodium stearoyl glutamate, coconut oil fatty acid acyl glutamic acid, potassium coconut oil fatty acid acyl glutamate, sodium coconut oil fatty acid acyl glutamate, lauroyl glutamic acid, potassium lauroyl glutamate, sodium lauroyl glutamate, myristoyl glutamic acid, potassium myristoyl glutamate, sodium myristoyl glutamate, stearoyl glutamic acid, potassium stearoyl glutamate, disodium stearoyl glutamate, sodium hydrogenated beef tallow fatty acid acyl glutamate, sodium coconut oil fatty acid/hydrogenated beef tallow fatty acid acyl glutamate, lauroyl methyl alanine, sodium lauroyl methyl alanine, sodium myristoyl methyl alanine, sodium lauroyl methyl taurate, sodium oleoyl methyl taurate, sodium alkane sulfonate, sodium tetradecene sulfonate, sodium dioctyl sulfosuccinate, disodium lauryl sulfosuccinate, sodium coconut oil fatty acid ethyl ester sulfonate, sodium lauryl sulfate, triethanolamine lauryl sulfate, sodium cetyl sulfate, triethanolamine alkyl sulfates, sodium alkyl sulfates, triethanolamine alkyl sulfates, alkyl ammonium sulfates, diethanolamine alkyl sulfates, triethanolamine alkyl sulfates, triethanolamine alkyl sulfates, lauryl ammonium sulfate, potassium lauryl sulfate, magnesium lauryl sulfate, monoethanolamine lauryl sulfate, diethanolamine lauryl sulfate, sodium myristyl sulfate, sodium stearyl sulfate, sodium oleyl sulfate, triethanolamine oleyl sulfate, sodium polyoxyethylenes lauryl ether sulfates, triethanolamine polyoxyethylene lauryl ether sulfate, sodium polyoxyethylene alkyl ether sulfates, triethanolamine polyoxyethylene alkyl ether sulfates, sodium polyoxyethylene myristyl ether sulfates, sodium higher fatty acid alkanolamide sulfate esters, lauryl phosphate, sodium lauryl phosphate, potassium cetyl phosphate, diethanolamine cetyl phosphate, polyoxyethylene oleyl ether phosphate, polyoxyethylene lauryl ether phosphate, sodium polyoxyethylene lauryl ether phosphate, polyoxyethylene cetyl ether phosphate, sodium polyoxyethylene cetyl ether phosphate, polyoxyethylene stearyl ether phosphate, polyoxyethylene oleyl ether phosphate, sodium polyoxyethylene oleyl ether phosphate, polyoxyethylene alkylphenyl ether phosphates, sodium polyoxyethylene alkylphenyl ether phosphates, triethanolamine polyoxyethylene alkylphenyl ether phosphates, polyoxyethylene octyl ether phosphate, polyoxyethylene alkyl ether phosphate, triethanolamine polyoxyethylene lauryl ether phosphate, and diethanolamine polyoxyethylene oleyl ether phosphate.

Exemplary cationic surfactants include alkyl amines, alkyl imidazolines, ethoxylated amides, quaternary compounds, quaternized esters, and alkyl amine oxides. Examples include lauramine oxide, dicetyldimonium chloride, and cetrimonium chloride.

Exemplary amphoteric and zwitterionic surfactants include betaines, alkyl amidopropyl betaines, alkyl sulfobetaines, alkyl glycinates, alkyl carboxyglycinates, alkyl amphopropionates, alkyl amidopropyl hydroxysultaines, acyl taurates and acyl glutamates wherein the alkyl and acyl groups have from 8 to 18 carbon atoms. Examples include cocoamidopropyl betaine, sodium cocoamphoacetate, cocoamidopropyl hydroxysultaine, and sodium cocoamphopropionate.

Exemplary nonionic surfactants include aliphatic (C₆-C₁₈) primary or secondary linear or branched chain acids, alcohols or phenols, alkyl ethoxylates, alkyl phenol alkoxylates (especially ethoxylates and mixed ethoxy/propoxy), block alkylene oxide condensate of alkyl phenols, alkylene oxide condensates of alkanols, ethylene oxide/propylene oxide block copolymers, semi-polar nonionics (e.g., amine oxides), as well as alkyl amine oxides. Other suitable nonionics include mono- or di-alkyl alkanolamides and alkyl polysaccharides, sorbitan fatty acid esters, polyoxyethylene sorbitan fatty acid esters, polyoxyethylene sorbitol esters, polyoxyethylene acids, and polyoxyethylene alcohols. Examples of nonionic surfactants include alkyl polyglucoside, cocamidopropyl and lauramine oxide, polysorbate 20, ethoxylated linear alcohols, cetearyl alcohol, lanolin alcohol, stearic acid, glyceryl stearate, polyoxyethylene lauryl ether, polyoxyethylene oleyl ether, PEG-100 stearate, sorbitan monooleate, sorbitan isostearate, and oleth-20, and mixtures thereof.

Exemplary powdered fillers include kaolin, silicic anhydride, magnesium aluminum silicate, sericite, talc, boron nitride, mica, montmorillonite, cellulose powder, wheat starch, silk powder, maize starch, and mixtures thereof.

Exemplary dyes and pigments include nitro dyes, azo dyes, nitroso dyes, xanthene dyes, quinoline dyes, anthraquinone dyes, indigo dyes, sepia powder, caramel, cochineal, carbon black, yellow iron oxide, black iron oxide, red iron oxide, titanium oxide, titanium dioxide, and mixtures thereof.

Exemplary pH adjusting agents include sodium hydroxide, potassium hydroxide, triethanolamine, and mixtures thereof.

Exemplary salts include sodium chloride, potassium chloride, magnesium chloride, sodium sulfate, and mixtures thereof.

Exemplary α-hydroxy acids include citric acid, glycolic acid, tartaric acid and lactic acid, and mixtures thereof.

Exemplary cosmetic and/or absorbent and/or body odor masking deodorant and/or antiperspirant agent, perfuming substance and/or perfumed oils include the complex zinc salt of ricinoleic acid, Styrax, derivatives of abiotic acid, sage essence, chamomile essence, carnation essence, lemon balm essence, mint essence, cinnamon leaf essence, lime flower essence, juniper berry essence, vetiver essence, olibanum essence, galbanum essence, labdanum essence, lavender essence, peppermint essence, bergamot orange, dihydromyrcenol, lilial, lyral, citronellol, lemon essence, mandarin essence, orange essence, lavender essence, muscat, geranium bourbon essence, aniseed, cilantro, cumin, juniper, extracts of fleur-de-lis, lilac, roses, jasmine, bitter orange blossom; benzyl acetate, p-tert-butylcyclohexyl acetate, linalyl acetate, phenylethyl acetate, ethylmethylphenyl glycinate, linalyl benzoate, benzyl formate, allyl cyclohexyl propionate, styrallyl propionate, benzyl salicylate, benzyl ethyl ether, linear alkanes with from 8 to 18 carbon atoms, citral, ricinoleic acid, citronellal, citronellyl oxyacetaldehyde, cyclamen aldehyde, hydroxycitronellal, bourgeonal, ionones, methyl cedryl ketone, anethole, eugenol, isoeugenol, geraniol, linalool, terpineol, phenylethyl alcohol, α-hexylcinnamaldehyde, geraniol, benzylacetone, cyclamen aldehyde, hydroxycitronellal, ambroxan, indole, hedione, sandelice, cyclovertal, β-damascone, allyl amyl glycolate, dihydromyrcenol, phenoxyethyl isobutyrate, cyclohexyl salicylate, phenylacetic acid, geranyl acetate, irotyl, floramate, active astringent products such as aluminum chloride, aluminum chlorohydrate, aluminum dichlorohydrate, aluminum sesquichlorohydrate, aluminum dihydroxyallantoinate, aluminum chlorotartrate, aluminum and zirconium trichlorohydrate, aluminum and zirconium tetrachlorohydrate, aluminum and zirconium pentachlorohydrate and/or mixtures thereof.

Exemplary essential oils include Archangelica officinalis (angelica) oil, Canangium odoratum (ylang ylang) oil, Canarium luzonicum (elemi) oil, orange oil, Chamomilla recutita (matricaria) oil, Anthemis nobilis oil, Elettaria cardamomum (cardamom) oil, Acorus calamus (calamus) oil, Ferula galbaniflua (galbanum) oil, Cinnamomum camphora (camphor) oil, Daucus carota (carrot) seed oil, Salvia sclarea (clary sage) oil, Citrusparadisi (grapefruit) oil, Eugenia caryophyllus (clove) oil, Cinnamon bark oil, Coriandrum sativum (coriander) oil, Cupressus sempervirens (cypress) oil, Santalum album (sandalwood) oil, Juniperus virginiana (cedar wood) oil, Cymbopogon nardus (citronella) oil, Cinnamomum verum (Cinnamon) leaf oil, Jasmine officinale (jasmine) absolute oil, Juniperus communis (juniper Berry) oil, Zingiber officinale (ginger) extract, Mentha spicata (spearmint) oil, Salvia officinalis (sage) oil, cedar oil, Pelargonium graveolens (geranium) oil, Thymus vulgaris (thyme) oil, Melaleuca alternifolia (tea tree) oil, Myristica fragrans (nutmeg) oil, Melaleuca viridiflora (niaouli) oil, Citrus aurantium (neroli) oil, pine oil, Ocimum basilicum (basil) oil, Mentha arvensis oil, Pogostemon cablin (patchouli) oil, Cymbopogon martinii (palmarosa) oil, Foeniculum vulgare (fennel) oil, Citrus bigaradia (petitgrain) oil, Piper nigrum (black pepper) oil, Boswellia carteri (frankincense) oil, Chrysopogon zizanioides (vetiver) oil, Mentha piperita (peppermint) oil, Citrus bergamia (bergamot) oil, benzoin oil, Aniba rosaeodora (rosewood) oil, Origanum majorana (marjoram) oil, mandarin oil, Commiphora myrrha (myrrh) oil, Melissa officinalis (balm mint) oil, Eucalyptus globulus oil, Citrus junos oil, Citrus aurantiifolia (lime) oil, Ravensara aromatica (clove) oil, Lavandula latifolia (lavandin) oil, Lavandula angustifolia (lavender) oil, Tilia vulgaris (linden) oil, lemon oil, lemon grass oil, rose oil, Aniba rosaeodora (rosewood) oil, Rosmarinus officinalis (rosemary) oil and Levisticum officinale (lovage) oil, and mixtures thereof.

In one embodiment, the active agent includes at least one active agent which is selected from skin whitening or depigmentation agents, anti-acne agents, and mixtures thereof.

Other pharmaceutical active ingredients and/or adjuvants useful herein include antacids; agents against peptic ulcers (e.g., butylscopolamine bromide, pirenzepine hydrochloride, timepidium bromide) and gastroesophageal reflux disease; antispasmodics; analgesics; anticholinergic drugs; propulsive drugs; antiemetics; anti-nausea drugs; agents for biliary therapy; agents for hepatic therapy; lipotropics; laxatives; antidiarrhetics; intestinal adsorbents; antipropulsives; anti-inflammatory drugs; active ingredients against obesity; enzymes; hypoglycemic drugs; insulin and analogues; vitamins; proteins; minerals; anabolic steroids; antithrombotic agents; antifibrinolytics; hemostatic agents; antiarrhythmic agents; cardiac stimulants; cardiac glycosides; vasodilators; antiadrenergic agents; antihypertensive drugs; diuretics; potassium-saving agents; antihemorrhoidals; antivaricose therapy agents; capillary stabilizing agents; agents which act on the renin-angiotensin system; beta-blockers; selective calcium-channel blockers; non-selective calcium-channel blockers; ACE inhibitors; angiotensin II inhibitors; agents modifying lipids; antifungals; antipruritics; anesthetics; antipsoriatics; chemotherapy drugs; corticosteroids; products for gynecological use (e.g., oxytocics, contraceptives, androgen, estrogen, progestogen, ovulation stimulants, gonadotropins, antiandrogens); products for urological use; antispasmodics; drugs used in benign prostatic hypertrophy; hormones; hormone antagonists; antibiotics; tetracyclines; amphenicols; penicillin; sulfonamides; trimethoprim; macrolides; lincosamides; streptogramins; antibacterial aminoglycosides; antibacterial quinolones; antivirals; immune serum; immunoglobulins; antineoplastic agents; immunomodulatory agents; alkylation agents; antimetabolites; plant alkaloids and other natural products; cytotoxic antibiotics; immunosuppressive agents; drugs for disorders of the musculoskeletal system; antirheumatics; agents which affect bone structure and mineralization; drugs which act on the nervous system; general anesthetics; local anesthetics; opioids; antimigraine agents; anticonvulsants; dopaminergic agents; antipsychotics (e.g., chlorpromazine hydrochloride, levomepromazine hydrochloride, clocapramine hydrochloride); anxiolytics; hypnotics; sedatives; antidepressants (e.g., imipramine hydrochloride, trazodone hydrochloride, fluvoxamine maleate); psychostimulants; anti-dementia drugs (e.g., donepezil, rivastigmine, galanthamide hydrobromide, memantine hydrochloride); antianxiety drugs (e.g., diazepam, alprazolam, tandospirone citrate); tranquilizers (hydroxyzine hydrochloride); brain function stimulant/activators (e.g., tiapride hydrochloride, protirelin tartrate); cerebral circulation improving drugs (isosorbide mononitrate or dinitrate, pentoxifylline, fasudil hydrochloride); Parkinson's disease therapeutic agents (hydrochloric acid benserazide, amantadine hydrochloride, talipexole hydrochloride); chemical-transmitter release-inhibition drugs (emedastine fumarate, suplatast tosilate, epinastine hydrochloride); cardiac disease therapeutic-agents (e.g., aminophylline, diltiazem hydrochloride, nicorandil, propranolol hydrochloride, isoprenaline hydrochloride, disopyramide phosphate, procainamide hydrochloride); antihypertensive drugs (e.g., captopril, enalapril maleate, amosulalol hydrochloride, prazosin hydrochloride, urapidil, clonidine hydrochloride); vasodilators (e.g., tolazoline hydrochloride); vasoconstrictors (e.g., amezinium metilsulfate, etilefrine hydrochloride, phenylephrine hydrochloride, midodrine hydrochloride); antihyperlipidemic drugs (pravastatin sodium, fluvastatin sodium, cerivastatin sodium); parasympathomimetics; drugs used in addictive disorders; anti-vertigo agents; antiparasitic agents; insecticides; insect repellants; nasal decongestants; antitussives and expectorants (dextromethorphan hydrobromide, fominoben hydrochloride, acetylcysteine); asthma preparations (clenbuterol hydrochloride, fenoterol hydrobromide, procaterol hydrochloride); mucolytic agents; cough suppressants; ophthalmic active ingredients; otological active ingredients; antiglaucoma drugs; miotics; mydriatics; cycloplegics; anti-dandruff agents; muscle contraction inhibitory agents; H2 blockers (e.g., ranitidine hydrochloride, roxatidine-hydrochloride acetate); proton pump inhibitors (e.g., omeprazole, lansoprazole, rabeprazole), antiemetic (e.g., granisetron hydrochloride, azasetron hydrochloride, ondansetron hydrochloride, ramosetron hydrochloride), anti-rheumatism agents (e.g., bucillamine, penicillamine); urological-diseases drugs (e.g., oxybutynin hydrochloride, tamsulosin hydrochloride, propiverine hydrochloride); (beta)-blockers (e.g., bisoprolol fumarate, betaxolol hydrochloride); and mixtures thereof.

The nature of these active ingredients excipients can be synthetic or natural, such as vegetable extracts, or come from a biotechnological process or from a combination of a synthetic process and a biotechnological process. Additional examples can be found in the CTFA International Cosmetic Ingredient Dictionary & Handbook, 12th Edition (2008). A biotechnological process is understood to be any process which produces the active ingredient, or part of it, in an organism, or in a part of it.

One class of substances are the therapeutic aids which include, but are not limited to, moisturizers (or things that help the substrate (skin) retain water); oils (or things that help the skin retain oil); pharmaceutical agents; antimicrobial agents; antibacterial agents; fungicide; anti-inflammatory/analgesic agents (e.g., things that reduce irritation); softening agents; toughening agents; agents that enhance elasticity of the substrate; agents that promote cell growth or cell reproduction; agents that retard cell growth or cell reproduction; stimulants for the cells or nerves, antihistamines; local anesthetics; and the like.

The hydrogel may include one or more active ingredients with one or more of the following advantages: sustained delivery, consistency in dosage, enhanced delivery, dosage control, efficiency, and bioavailability for: wound healing, burn healing, scar reducing, etc.; skin or keratin color changes (lightening, darkening, coloring), applying decorative images, highlighting; enhancing penetration of another active ingredient or medicine through the skin or other substrate; altering the fragrance or aroma of the substrate, or enhancing fat, e.g., cellulite reduction; applying a hormone, steroid, or pheromone, etc.

In one embodiment, the clarity and/or appearance of the hydrogels of the invention can be adjusted. The clarity of the hydrogels may vary from substantially transparent, with little visual haze, to where insoluble component additives such as beads, pearlizing agents, are clearly visible to visually opaque. The hydrogels may incorporate long-term suspension of particles, insoluble liquid droplets. The materials or compounds which may be suspended can be soluble or insoluble. In some embodiments, the hydrogel is opacified by deliberately incorporating pearlescent materials therein to achieve an attractive pearl-like appearance, known as pearlescence. Examples of such other insoluble compounds include pigments, minerals such as bismuth, antimicrobials such as silver or zinc particles, dyes, and the like.

### INDUSTRIAL APPLICATION

Some embodiments of the invention relate to the use of the hydrogels as multi-functional polymer ingredients in personal care, health care, household, institutional and industrial product applications and the like. The hydrogels can be employed as emulsifiers, spreading aids and carriers for enhancing the efficacy, deposition and delivery of chemically and physiologically active ingredients and cosmetic materials, and as a vehicle for improving the psychosensory and aesthetic properties of a formulation in which they are included. The term “personal care products” as used herein includes, without limitation, cosmetics, toiletries, cosmeceuticals, beauty aids, personal hygiene and cleansing products that are applied to the skin, hair, scalp, and nails of humans and animals. The term “health care products” as used herein includes, without limitation, pharmaceuticals, pharmacosmetics, oral care products (mouth, teeth), eye care products, ear care products and over-the-counter products and appliances, such as patches, plasters, dressings and the like. The term also includes medical devices that are externally applied to or into the body of humans and animals for ameliorating a health related or medical condition. The term “body” includes the keratinous (hair, nails) and non-keratinous skin areas of the entire body (face, trunk, limbs, hands and feet), the tissues of body openings and the eyes. The term “skin” includes the scalp and mucous membranes.

The hydrogel as described herein can be utilized in various forms, including but not limited to a gel, a single layer sheet, a sheet on a barrier film. The hydrogel composition may further include an article which is a medical article, a personal care article, a pharmaceutical article or a health care article. The medical article can include, but is not limited to, a wound covering, a dressing, a controlled drug delivery device, a component in a more complex device, such as a biosensor or microfluidic device. In some embodiments, the wound covering can include a backing and a facing, or may take the form of a sheet, a gel or an impregnated gauze. In one embodiment, the hydrogel composition includes a transdermal, dermal or mucosal delivery agent for the delivery of a chemically or physiologically active ingredient.

The amount of each chemical component described is presented exclusive of any solvent, which may be customarily present in the commercial material, that is, on an active chemical basis, unless otherwise indicated. However, unless otherwise indicated, each chemical or composition referred to herein should be interpreted as being a commercial grade material which may contain the isomers, by-products, derivatives, and other such materials which are normally understood to be present in the commercial grade.

It is known that some of the materials described above may interact in the final formulation, so that the components of the final formulation may be different from those that are initially added. For instance, metal ions (of, e.g., a detergent) can migrate to other acidic or anionic sites of other molecules. The products formed thereby, including the products formed upon employing the composition of the present invention in its intended use, may not be susceptible of easy description. Nevertheless, all such modifications and reaction products are included within the scope of the present invention; the present invention encompasses the composition prepared by admixing the components described above.

### EXAMPLES

The invention will be further illustrated by the following examples, which sets forth particularly advantageous embodiments. While the examples are provided to illustrate the present invention, they are not intended to limit it. Unless otherwise specified weight percents (wt. %) are given in wt. % based on the weight of the total composition.

### Test Methods

### Rheological Measurements

Hydrogels have been tested using a TA instrument ARES controlled strain rheometer using 1″ parallel plates, 1 mm gap, using a dynamic strain sweep from 0.01% to 625% —1 Pa to 5000 Pa at 1 Hz at RT (23° C.). The strain rate was controlled and the shear stress necessary to achieve it was calculated from the torque. A known mucilage of polymer as prepared in example is centrifuged to remove any bubbles or a compressed sheet without bubbles is used. Sample mucilage is loaded on the bottom plate and excess is removed. The flow curve program is started and data collected under increasing levels of steady shear. From this, Elastic modulus (G′), Viscous modulus (G″) and Tan δ and Yield Stress are calculated:

Elastic (Storage) Modulus, measure of the material ability to store energy=G′=(Stress/Strain)cos δ

Viscous (Loss) Modulus, measure of the ability of the material to dissipate energy=G″=(Stress/Strain)sin δ

Tan Delta, measure of the material ability to damp, adhesion properties, and tackiness=Tan δ=G″/G′

Yield Stress where the two line of G″ G′ cross. In many of these samples, the G′ and G″ do not cross. Therefore the value reported as Yield stress is the peak shear stress point in the plot of shear stress vs. shear strain curve.

The choice of the TPU component, and the ratio between the poly(acrylic) acid polymer and the TPU component, as well as the degree of neutralization of the poly(acrylic) acid polymer will each have an impact on the physical properties of the resulting hydrogel. These parameters may be used to select the combination of the properties desired in the resulting hydrogel. For this reason, hydrogels have been tested using a TA instrument ARES controlled strain rheometer using 2″ parallel plates at RT and in a range from 1 to 1000 sec⁻¹. A few of the more important physical properties as found in rheological testing are further commented on below.

### Materials

The materials are generally commercially available from chemical supply houses known to those skilled in the chemical arts or from the supplier indicated below.

| | |
|---|---|
| Carbopol ® 980NF | Carbomer homopolymer Type C available from |
| | The Lubrizol Corporation |
| Carbopol 981 NF | Carbomer homopolymer Type A available from |
| | The Lubrizol Corporation |
| Carbopol ETD 2020 | Carbomer Interpolymer Type B available from |
| | The Lubrizol Corporation |
| Euxyl-PE9010 | liquid preservative based on phenoxyethanol and |
| | ethylhexylglycerin available from Schulke, Inc. |
| PolyOx WSR-301 | polyethylene oxide available from Colorcon having |
| | a molecular weight of 1 × 10⁵ to 7 × 10⁶ |
| TPU1 | a water soluble aliphatic polyether thermoplastic |
| | polyurethane |
| | available from The Lubrizol Corporation |
| TPU2 | a water swellable aliphatic polyether thermoplastic |
| | polyurethane |

### Example 1

### Method 1: Powder TPU1 Addition to Carbopol® Gel

2 gm of Carbopol® 980 is dispersed into 382 gm water and blended for 15 minutes after dispersion with Hobart mixer. Disperse 2 gm of Euxyl PE9010. The dispersion is then neutralized with 1.5 gm 18 wt % NaOH to bring the pH to 5.0+/−0.5. The gel is allowed hydrate at room temperature for 1 hour. Following hydration 16 gm of powder TPU1 to the gel. In Hobart, mix for approximately 15 minutes or until well mixed. Allow to hydrate overnight and mix 5 minutes. Measure the final pH 5.0+/−0.5. The final concentration of Carbopol 980 is 0.5 wt % and MPD 344 is 4.5 wt % and a total polymer content 4.5. An extremely thick homogeneous gel is formed. The gel is measure by Rheology for G′ at 100 Pa, G″ at 100 Pa, Tan δ and Yield Stress Pa. The gel is stiff and bouncy and can be compression molded at 50° C. into ⅛ and ¼″ plaques. Table 2 illustrates mixtures made by method 1.

TABLE 2

| | | | | | | final |
|---|---|---|---|---|---|---|
| | Gm | Gm | Gm | Final | Final | total |
| Example | TPU | water | Cbpl | Cbpl | TPU | Poly |
| Inv. Ex 1 | 16 | 382 | 2 | 0.5% | 4.0% | 4.5% |
| Inv. Ex 2 | 12 | 388 | 4 | 1.0% | 3.0% | 4.0% |
| Inv. Ex 3 | 16 | 380 | 4 | 1.0% | 4.0% | 5.0% |
| Inv. Ex 4 | 8 | 388 | 4 | 1.0% | 2.0% | 3.0% |
| Inv. Ex 5 | 8 | 98.00 | 2 | 0.5% | 2.0% | 2.5% |

TABLE 3
Properties of Table 2 Compositions

| | G′ | G″ | | | |
|---|---|---|---|---|---|
| | At | At | | Yield | |
| | 10%, | 10% | | Stress | |
| Example | Pa | Pa | Tan δ | Pa | Appearance |
| Inv. Ex 1 | 3656 | 828 | 0.23 | 6054 | Stiff and |
| | | | | | bouncy |
| Inv. Ex 2 | | | | | Resilient |
| | | | | | and clear |
| Inv. Ex 3 | 2128 | 513 | 0.24 | 4680 | Acceptable |
| Inv. Ex 4 | 1740 | 364 | 0.21 | 3545 | Weak |
| Inv. Ex 5 | | | | | Weak |

### Example 2

### Method 2: Dissolving of TPU1 in Water and Addition to Partially Neutralized Carbopol® Gel

8 gm of TPU1 is dissolved in 292 gm water to reach a wt % of 2.67%. In a Hobart Mixer 100 gm of 2 wt % Carbopol acid dispersion is pH adjusted with approximately 2.3 gm 18 wt % NaOH or until pH reaches 5.5. The TPU solution is added to the neutralized Carbopol solution and mixed with Hobart mixer for approximately. 15 minutes. Final concentration 0.5 wt % Cbpl and 2.0 wt % TPU and total polymer 2.50 wt %.

TABLE 4

| | gm TPU | Wt % | Cbpl | | Final | Final | final | |
|---|---|---|---|---|---|---|---|---|
| Example | solution | TPU | solution | wt % | Cbpl | TPU | total | pH |
| Inv. Ex 6 | 300 | 2.67 | 100 | 2% | 0.5% | 2.0% | 2.5% | 5.5 |

TABLE 5
Properties of Table 4 Compositions

| | G′ | G″ | | | |
|---|---|---|---|---|---|
| | At | At | | Yield | |
| | 100 Pa, | 100 Pa, | | Stress | |
| Example | Pa | Pa | Tan δ | Pa | Appearance |
| Inv. Ex 6 | | | | | Weak |

### Example 3

### Method 3 Addition of Carbopol Acid Dispersion to High pH TPU

Part A: A dispersion of Carbopol 980 is NF polymer is made using 776 gm of water to which 24 gm of Carbopol 980 NF polymer is added and stirred for 15 minutes and allow to hydrate for 1 hour to provide a 3 wt % solution.

Part B: A solution of TPU1 in water is made by adding 253 gm water to 14 gm TPU1 to provide a 5.3 wt % solution. 2 gm of Euxyl PE9010 is added and the mixture is agitated until the TPU is completely in solution.

In a Hobart mixer, 267 gm of Part B is added and blended for approximately 15 minutes or until well mixed. Add 3.09 gm 18 wt % NaOH, or appropriate amount to partially neutralize the recipe amount of Carbopol to approx. 0.25 degree of neutralization (pH 12.5). This mixture is then added to 67 gm of Part A and stirred for approximately 15 minutes or until well mixed. The mixtures is allowed to hydrate overnight and the final pH is measured. The final product is an extremely thick homogeneous gel. The gel is measure by Rheology for G′ at 100 Pa, G″ at 100 Pa, Tan δ and Yield Stress Pa. The gel is stiff and bouncy and can be compression molded at 50° C. into 0.32 cm and 0.64 cm plaques. Table 6 illustrates examples made in this manner.

TABLE 6

| | Sample | | | | | | | |
|---|---|---|---|---|---|---|---|---|
| | Part A | | Part B | | Final | | | |
| | Cbpl | Conc. | TPU 1 | TPU 1 | Final | Final | final | |
| | sol. | Cbpl | sol. | Conc. | Cbpl | TPU | total | final |
| Ex. | gm | wt % | gm | wt % | wt % | wt % | wt % | pH |
| Inv. Ex 7 | 100 | 3.0% | 300 | 6.0% | 0.8% | 4.5% | 5.3% | 5.05 |
| Inv. Ex 8 | 133 | 3.0% | 267 | 5.3% | 1.0% | 3.5% | 4.5% | 5 |
| Inv. Ex 9 | 171 | 4.7% | 228 | 5.3% | 2.0% | 3.0% | 4.9% | N/R |
| Inv. Ex 10 | 229 | 5.2% | 171 | 4.7% | 2.9% | 2.0% | 4.9% | N/R |
| Inv. Ex 11 | 145 | 4.2% | 255 | 4.7% | 1.5% | 3.0% | 4.5% | 4.75 |
| Inv. Ex 12 | 33 | 3.0% | 367 | 4.4% | 0.3% | 4.0% | 4.3% | 5.4 |
| Inv. Ex 13 | 133 | 3.0% | 267 | 4.5% | 1.0% | 3.0% | 4.0% | 4.91 |
| Inv. Ex 14 | 133 | 3.0% | 267 | 4.5% | 1.0% | 3.0% | 4.0% | N/R |
| Inv. Ex 15 | 200 | 3.0% | 200 | 5.0% | 1.5% | 2.5% | 4.0% | N/R |
| Inv. Ex 16 | 67 | 3.0% | 333 | 3.6% | 0.5% | 3.0% | 3.5% | 5.1 |
| Inv. Ex 17 | 157 | 2.0% | 253 | 4.0% | 1.0% | 2.5% | 3.5% | N/R |
| Inv. Ex 18 | 67 | 3.0% | 333 | 4.0% | 0.5% | 3.0% | 3.5% | 5.18 |
| Inv. Ex 19 | 100 | 3.0% | 300 | 3.3% | 0.8% | 2.5% | 3.3% | 5 |
| Inv. Ex 20 | 233 | 3.0% | 167 | 3.6% | 1.8% | 1.5% | 3.3% | 4.01 |
| Inv. Ex 21 | 133 | 3.0% | 267 | 3.0% | 1.0% | 2.0% | 3.0% | 5.92 |
| Inv. Ex 22 | 67 | 3.0% | 333 | 2.4% | 0.5% | 2.0% | 2.5% | 5.11 |
| Inv. Ex 23 | 100 | 3.0% | 300 | 2.0% | 0.8% | 1.5% | 2.3% | 4.97 |

TABLE 7

| | G′ | G″ | | |
|---|---|---|---|---|
| | (at | (at | | Yield |
| | 100 Pa) | 100 Pa) | | Stress |
| Example | (Pa) | (Pa) | Tan δ | Pa |
| Inv. Ex 7 | 3588 | 884 | 0.25 | 8189 |
| Inv. Ex 8 | 3075 | 653 | 0.21 | 5474 |
| Inv. Ex 9 | 3668 | 852 | 0.24 | 8765 |
| Inv. Ex 10 | 3089 | 662 | 0.21 | 7380 |
| Inv. Ex 11 | 2136 | 537 | 0.25 | 5790 |
| Inv. Ex 12 | 2248 | 660 | 0.29 | 5557 |
| Inv. Ex 13 | 2608 | 558 | 0.21 | 5644 |
| Inv. Ex 14 | 3782 | 799 | 0.21 | 7165 |
| Inv. Ex 15 | 1582 | 346 | 0.22 | 5258 |
| Inv. Ex 16 | 2106 | 481 | 0.23 | 4470 |
| Inv. Ex 17 | 1225 | 320 | 0.26 | 4016 |
| Inv. Ex 18 | 1815 | 459 | 0.25 | 3716 |
| Inv. Ex 19 | 1676 | 386 | 0.23 | 4100 |
| Inv. Ex 20 | 1921 | 338 | 0.18 | 4495 |
| Inv. Ex 21 | 2307 | 447 | 0.19 | 4362 |
| Inv. Ex 22 | 1163 | 298 | 0.26 | 2716 |
| Inv. Ex 23 | 948 | 227 | 0.24 | 2761 |

As can be seen in the above Table 7, the inventive examples decrease in total concentration from a high total polymer content of 5.3 to a low polymer content of 2.3 and all show a Yield Stress above 2,500 Pa.

### Example 4

TPU Blends: Blend of Water Soluble TPU and Other TPU that is Ethanol Soluble

Part A: A 3 wt % dispersion of Carbopol 980 is made to which 0.5 wt % of Euxyl PE9010 is added and stirred for 15 minutes. This mixture is allowed to hydrate for 1 hour.

Part B: 4.8 gm of TPU1 is dissolved in 189.2 gm water. To this, 6 gm of 20 wt % solution of TPU2 and blended for 15 minutes, followed by the addition of 2.3 gm of 18 wt % NaOH.

Part B is then added to the recipe amount of Part A as described in Table 8 and stirred for approximately 15 min or until well mixed and allowed to hydrate overnight and the final pH is measured.

TABLE 8

| | Part A | | Part B | | | Final | | | |
|---|---|---|---|---|---|---|---|---|---|
| | Cbpl | | TPU | | Wt % | Cbpl | | | |
| Exam- | sol | Wt % | sol | Wt % | TG- | Wt | | TG- | final |
| ple | gm | Cbpl | Gm | 344 | 2000 | % | 344 | 2000 | total |
| Inv. | 100 | 3% | 200 | 2.4 | 0.6 | 1 | 1.6 | 0.4% | 3 |
| Ex. | | | | | | | | | |
| 24 | | | | | | | | | |

TABLE 9
Properties of Table 8 Composition

| | G′ | G″ | | | |
|---|---|---|---|---|---|
| | (at | (at | | | |
| | 100 Pa) | 100 Pa) | | Yield | |
| Example | (Pa) | (Pa) | Tan δ | Stress Pa | Appearance |
| Inv. Ex 24 | 1956 | 336 | 0.17 | 4092 | |

### Comparative Example 1

### Method 3: Addition of Carbopol Acid Dispersion to High pH PEO

Part A: A dispersion of Carbopol 980 at the concentration as specified in Table 9 below is made and stirred for 15 minutes and then allowed to hydrate for 1 hour.

Part B: A solution of WSR 301 PEO in water at the concentration as specified in Table 9 below is made and 0.5 wt % of Euxyl PE9010 is added. The mixture is agitated until the PEO is completely in solution.

In Hobart mixer, recipe amount as described in Table 9 below of Part B is blended for approximately 15 minutes or until well mixed followed by addition of 18 wt % NaOH or an appropriate amount to partially neutralize the recipe amount of Carbopol to approximately a 0.25 degree of neutralization.

To above recipe, an amount as described in Table of Part A is added and stirred for approximately 15 min or until well mixed. The mixture is allowed to hydrate overnight and final pH is measured.

TABLE 10
Comparative Examples

| | Part B | | Part A | | Final | | | Properties | | | |
|---|---|---|---|---|---|---|---|---|---|---|---|
| | PEO | Conc. | Cbpl | Conc. | Final | Final | final | G′ (at | G″ (at | | Yield |
| | sol. | PEO | sol. | Cbpl | Cbpl | PEO | total | 100 Pa) | 100 Pa) | Tan | Stress |
| Ex. | gm | wt % | gm | wt % | wt % | wt % | wt % | Pa | Pa | δ | Pa |
| 24 | 200 | 8.0% | 200 | 3% | 1.5% | 4.0% | 5.5% | 2529 | 390 | 0.15 | 1514 |
| 25 | 229 | 5.3% | 171 | 4.7% | 2.0% | 3.0% | 5.0% | 2603 | 417 | 0.16 | 1387 |
| 26 | 170 | 4.7% | 230 | 5.2% | 3.0% | 2.0% | 5.0% | 2268 | 332 | 0.15 | 1107 |
| 27 | 200 | 7.0% | 200 | 3% | 1.5% | 3.5% | 5.0% | 1996 | 310 | 0.16 | 1228 |
| 28 | 333 | 4.8% | 67 | 3% | 0.5% | 4.0% | 4.5% | 1685 | 284 | 0.17 | 1045 |
| 29 | 267 | 4.5% | 133 | 3% | 1.0% | 3.0% | 4.0% | 1887 | 311 | 0.16 | 925 |
| 30 | 133 | 6.0% | 276 | 3% | 2.0% | 2.0% | 4.0% | 1680 | 257 | 0.15 | 764 |
| 31 | 333 | 3.3% | 67 | 3% | 0.5% | 2.8% | 3.3% | 1355 | 267 | 0.20 | 690 |
| 32 | 200 | 3.0% | 200 | 3% | 1.5% | 1.5% | 3.0% | 1238 | 164 | 0.13 | 586 |

As can be seen in the above Table 10, identical concentrations of a high molecular weight polyethylene oxide polymer are blended in the same manner with the pH sensitive microgel (Carbopol) and at the same ratios as the inventive examples. The same rheological tests were conducted on these comparative example gels. It can be seen in the above Table 10 that the Yield Stress of the comparative examples at the same concentration to the inventive samples is significantly lower (as illustrated in FIG. 1). This was an unexpected result as the comparative example yield stress is expected for a blend of two water soluble polymers. However, the inventive examples, which are also a blend of two water soluble polymers, exhibit a much higher yield stress.

Without wishing to be bound by theory, it is believed that the water soluble thermoplastic polyurethane of this invention have hard segments that can reversibly “microphase separate” to form periodic nanostructures at lower temperature in the water. The hard segments act as thermally reversible crosslinks. In this hydrogel this nanostructure gives surprising Yield Stress to the hydrogel. This can be seen in the comparative examples of Polyethylene Oxide which does not have this microphase separation. This can be seen in the ability to heat the hydrogel to between 50° C. and 80° C. and extrude through a die to form a sheet.

Each of the documents referred to above is incorporated herein by reference, including any prior applications, whether or not specifically listed above, from which priority is claimed. The mention of any document is not an admission that such document qualifies as prior art or constitutes the general knowledge of the skilled person in any jurisdiction. Except in the Examples, or where otherwise explicitly indicated, all numerical quantities in this description specifying amounts of materials, reaction conditions, molecular weights, number of carbon atoms, and the like, are to be understood as modified by the word “about.” It is to be understood that the upper and lower amount, range, and ratio limits set forth herein may be independently combined. Similarly, the ranges and amounts for each element of the invention can be used together with ranges or amounts for any of the other elements.

As used herein, the transitional term “comprising,” which is synonymous with “including,” “containing,” or “characterized by,” is inclusive or open-ended and does not exclude additional, un-recited elements or method steps. However, in each recitation of “comprising” herein, it is intended that the term also encompass, as alternative embodiments, the phrases “consisting essentially of” and “consisting of,” where “consisting of” excludes any element or step not specified and “consisting essentially of” permits the inclusion of additional un-recited elements or steps that do not materially affect the essential or basic and novel characteristics of the composition or method under consideration.

1. A dual network hydrogel composition comprising:
a) a poly(acrylic) acid crosslinked polymer derived from one or more olefinically unsaturated polymerizable carboxylic monomers; and
b) one or more thermoplastic polyurethane (TPU) polymers;
wherein the polymer content is from about 2.0 wt % to about 8 wt % of the total composition.

2. The hydrogel of claim 1, wherein the cross-linked polymer is a carbomer copolymer, a carbomer homopolymer, carbomer interpolymer, or a polycarbophil.

3. The hydrogel of claim 1, wherein the poly(acrylic) acid polymer is cross-linked with an allyl ether cross-linking agent or divinyl glycol.

4. The hydrogel of claim 3, wherein the allyl ether cross-linking agent comprises one or more of allyl pentaerythritol, allyl sucrose, or trimethpropanediolyl ether (TMPDE).

5. The hydrogel of claim 1, wherein the TPU polymer comprises the reaction product of (i) at least one aliphatic or aromatic diisocyanate; (ii) a polyol component comprising at least one polyether polyol having a number average molecular weight of at least 300; and (iii) optionally, a chain extender component.

6. The hydrogel of claim 5, wherein the chain extender comprises an aliphatic diol.

7. The hydrogel of claim 5, wherein the polyether polyol comprises polyethylene glycol, polypropylene glycol or combinations thereof

8. The hydrogel of claim 7, wherein the polyether polyol component comprises a blend of polyethylene glycol polyols having number average molecular weights (Mn) of at least 300 and at least 1450.

9. The hydrogel of claim 7, wherein the polyol component comprises a blend of polyethylene glycol polyols having number average molecular weights (Mn) of at least 1450 and at least 8000.

10. The hydrogel of claim 7, wherein the polyol component comprises a blend of polyethylene glycol and polypropylene glycol.

11. The hydrogel of claim 1 wherein the cross-linked polymer is partially neutralized.

12. The hydrogel of claim 1, further comprising a comonomer, the comonomer comprising one or more of at least one acrylic acid ester of the formula: 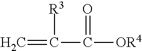
wherein R³ is hydrogen, methyl or ethyl and R⁴ is an alkyl group containing 1 to 30 carbon atoms, in an amount of less than 30 weight percent based upon the weight of the carboxylic acid or anhydride plus the acrylic acid ester.

13. The hydrogel of claim 1, wherein, wherein the ratio of the components (a) to (b) is from 0.5:1 to 10:1.

14. The hydrogel of claim 1, wherein the polymer content is from about 3.5 wt % to about 5 wt % of the total composition.

15. The hydrogel of claim 1, wherein the polymer content is at least 2 wt %.

16. The hydrogel of claim 1, wherein the poly(acrylic) acid cross-linked polymer is present in an amount of at least 0.5 wt % of the total composition.

17. The hydrogel of claim 13, wherein the poly(acrylic) acid cross-linked polymer is present in an amount from 0.5 wt % to 3 wt % of the total composition.

18. The hydrogel of claim 1, further comprising one or more of a pharmaceutical, a biologically active compound, an absorptive material, a personal care compound, an active ingredient, a therapeutic aid, or combinations thereof.

19. A wound covering comprising the hydrogel of claim 1.

20. The hydrogel composition of claim 1, wherein the hydrogel is in sheet form.

21. The hydrogel composition of claim 20, wherein the sheet has a thickness of from 0.2 cm to 0.7 cm.

22. The hydrogel of claim 1, wherein the TPU polymer comprises a water soluble TPU or a blend of a water soluble and water swellable TPU.

23. The hydrogel of claim 6, wherein the chain extender component comprises one or more of diethylene glycol or a C₃-C₁₂ diol and is present in an amount from 0.4 wt % to 4 wt %.

24. A dual network hydrogel composition comprising:
a) a crosslinked polymer derived from one or more olefinically unsaturated polymerizable carboxylic monomers;
b) an optional comonomer; and
c) a thermoplastic polyurethane (TPU) comprising the reaction product of:
i) an aliphatic or aromatic diisocyanate; and
ii) a polyol component comprising of at least one polyethylene glycol having a number average molecular weight (Mn) of at least 1450;
wherein the composition is thermoformable and semi-solid.

25. The hydrogel composition of claim 24, wherein the composition is thermoformable at temperatures of from about 50° C. to about 90° C.

26. The hydrogel composition of claim 24, wherein the total polymer content is at least 2.0 wt % of the total composition.

27. The hydrogel composition of claim 24, including the comonomer comprising one or more of at least one acrylic acid ester of the formula: 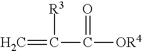
wherein R³ is hydrogen, methyl or ethyl and R⁴ is an alkyl group containing 1 to 30 carbon atoms, in an amount of less than 30 weight percent based upon the weight of the carboxylic acid or anhydride plus the acrylic acid ester.

28. The hydrogel composition of claim 27, wherein the comonomer is present in an amount from 0.5 wt % to 65 wt %.

29. The hydrogel composition of claim 24, wherein the polyol component of the TPU polymer comprises a blend of polyethylene glycol polyols having number average molecular weights (Mn) of at least 1450 and at least 8000

30. A dual network hydrogel composition comprising:
a) a homopolymer of a crosslinked polymer derived from one or more olefinically unsaturated polymerizable carboxylic monomers; and
b) a hydrophilic thermoplastic polyurethane polymer;
wherein the total polymer content of the composition is at least 2.0 wt % of the total composition.

31. The hydrogel composition of claim 30, wherein the hydrophilic thermoplastic polyurethane composition comprises (i) an aromatic diiosocyanate component; (ii) at least one polyether polyol component having a number average molecular weight of at least 300; and (iii) an optional chain extender component.

32. The hydrogel composition of claim 30, wherein the polymer content is from 2.0 wt % to 8 wt % of the total polymer composition.

33. An article including a dual network hydrogel composition the composition comprising:
a) a poly(acrylic) acid crosslinked polymer derived from one or more olefinically unsaturated polymerizable carboxylic monomers; and
b) one or more thermoplastic polyurethane (TPU) polymers;
wherein the polymer content is from about 2.5 wt % to about 6 wt % of the total composition.

34. The article of claim 33, wherein the article comprises a medical article.

35. The article of claim 34, wherein the medical article comprises one or more of a wound covering, a dressing, a controlled drug delivery device, a microfluidic device, or a biosensor.

36. The article of claim 35, wherein the wound covering includes a backing and a facing.

37. The article of claim 35, wherein the wound covering article is in the form of a sheet, a gel or an impregnated gauze.

38. The article of claim 33, wherein the article is a personal care article, a pharmaceutical article or a health care article.

39. A dermal, mucosal or transdermal delivery agent for the delivery of chemically and physically active ingredients comprising a dual network hydrogel composition comprising:
a) a poly(acrylic) acid crosslinked polymer derived from one or more olefinically unsaturated polymerizable carboxylic monomers; and
b) one or more thermoplastic polyurethane polymers;
wherein the polymer content of the hydrogel composition is from about 2.0 wt % to about 8.0 wt %.

40. A method of making a dual network hydrogel composition, said method comprising the step of: (I) reacting:
a) a crosslinked polymer derived from one or more olefinically unsaturated polymerizable carboxylic monomers; and
b) a thermoplastic polyurethane comprising the reaction product of:
i) a polyisocyanate; and
ii) a polyol component comprising of at least one polyethylene glycol polyol having a molecular weight (Mn) of at least 800; and
(iii) an optional chain extender;
wherein a) and b) are reacted at a ratio of 0.5:1 to 10:1.

41. A dual network hydrogel composition comprising:
a) a pH sensitive microgel cross-linked poly(acrylic) acid; and
b) one or more water soluble or water swellable thermoplastic polyurethane polymers.

42. The hydrogel composition of claim 41, wherein the hydrogel exhibits a Yield Stress of from 1000 to 7500 Pa and has pH sensitive microgel content of 0.25 to 3 wt %, a water soluble TPU from 1.5 to 4.5 wt % and an ethanol/water soluble TPU of 0 to 1 wt %.

43. The hydrogel composition of claim 41, wherein the hydrogel comprises a polymer content of at least 2.3 wt %, or at least 3 wt % of the total composition.

44. A dual network hydrogel composition comprising a mixture of (i) a pH sensitive microgel polyacrylic acid and (ii) a water soluble polyether thermoplastic urethane or a blend of a water soluble thermoplastic urethane and a water swellable thermoplastic polyurethane and the hydrogel has a Yield Stress of at least 2,500 Pa.

45. The hydrogel composition of claim 44, wherein the polymer content of the hydrogel is at the most 8 wt %, or at most 7 wt % or at most 6 wt % of the total composition.

46. The hydrogel composition of claim 44, wherein the water soluble or water swellable thermoplastic polyurethane polymers are the reaction product of i) a first water soluble polyether polyol having a molecular weight of at least 3000 daltons; (ii) a diisocyanate; and (iii) at least one of a second polyol having a molecular weight of up to 800 daltons, a third polyether polyol having a molecular weight of no more than 2500 daltons, or a chain extender.

47. The hydrogel composition of claim 44, wherein the water soluble or water swellable thermoplastic polyurethane polymers are the reaction product of i) a first water soluble polyether polyol having a molecular weight of at least 3000 daltons; (ii) a diisocyanate; and (iii) at least two of a second polyol having a molecular weight of up to 800 daltons, a third polyether polyol having a molecular weight of no more than 2500 daltons, or a chain extender.

48. A method of forming a hydrogel wound dressing, or a dermal delivery hydrogel comprising reacting a) a crosslinked polyacrylic acid microgel; and b) a thermoplastic polyurethane polymer which is water soluble or water swellable which includes the reaction product of (i) a first water soluble polyether polyol having a molecular weight of at least 3000 daltons; (ii) a diisocyanate; and (iii) at least one of a second polyol having a molecular weight of up to 800 daltons, a third polyether polyol having a molecular weight of no more than 2500 daltons, or a chain extender

49. The method of claim 48, wherein the microgel and TPU together with water form at least 2.0 wt % total polymer.

50. The method of claim 48 further comprising, adding an active agent to form a hydrogel sheet which includes the active agent dispersed in the hydrogel.

51. A hydrogel wound dressing or a dermal delivery hydrogel comprising at least 92 wt. % water, 1 to 5 wt. % of a thermoplastic polyurethane polymer, 0.5 to 4 wt % pH sensitive microgel and at least one active agent, wherein the polymer content of the hydrogel is at least 2.0 wt % of the total composition.

52. The hydrogel composition of claim 1, wherein a hard segment of the TPU comprises from 0.25 wt % to 6 wt %.

53. The hydrogel composition of claim 1, wherein the hard segment of the TPU comprises at least 0.25 wt %, or at least 0.35 wt %.

54. The hydrogel composition of claim 1, wherein the soft segment of the TPU comprises at least 80 wt %, or from 80 wt % to 95 wt %.
